SEQUENCE LISTING (46 sequences)

SEQ ID NO: 1 - DNA, 30 bp, synthetic construct
  misc_feature 1..30: note = Synthetic polynucleotide
  misc_feature 1..3: note = Backbone modified with phosphorothioate
  misc_feature 27..29: note = Backbone modified with phosphorothioate
  source 1..30: mol_type = other DNA
aaaaaaaaaaaaaaaaaaaaaaaaaaaaaa

SEQ ID NO: 2 - DNA, 30 bp, synthetic construct
  misc_feature 1..30: note = Synthetic polynucleotide
  misc_feature 1..3: note = Backbone modified with phosphorothioate
  misc_feature 27..29: note = Backbone modified with phosphorothioate
  source 1..30: mol_type = other DNA
tttttttttttttttttttttttttttttt

SEQ ID NO: 3 - DNA, 50 bp, synthetic construct
  misc_feature 1..50: note = Synthetic polynucleotide
  misc_feature 1..3: note = Backbone modified with phosphorothioate
  misc_feature 47..49: note = Backbone modified with phosphorothioate
  source 1..50: mol_type = other DNA
aaaaaaaaaaaaaaaaaaaaaaaaaaaaaaaaaaaaaaaaaaaaaaaaaa

SEQ ID NO: 4 - DNA, 50 bp, synthetic construct
  misc_feature 1..50: note = Synthetic polynucleotide
  misc_feature 1..3: note = Backbone modified with phosphorothioate
  misc_feature 47..49: note = Backbone modified with phosphorothioate
  source 1..50: mol_type = other DNA
tttttttttttttttttttttttttttttttttttttttttttttttttt

SEQ ID NO: 5 - DNA, 60 bp, synthetic construct
  misc_feature 1..60: note = Synthetic polynucleotide
  misc_feature 1..3: note = Backbone modified with phosphorothioate
  misc_feature 57..59: note = Backbone modified with phosphorothioate
  source 1..60: mol_type = other DNA
aaaaaaaaaaaaaaaaaaaaaaaaaaaaaaaaaaaaaaaaaaaaaaaaaaaaaaaaaaaa

SEQ ID NO: 6 - DNA, 60 bp, synthetic construct
  misc_feature 1..60: note = Synthetic polynucleotide
  misc_feature 1..3: note = Backbone modified with phosphorothioate
  misc_feature 57..59: note = Backbone modified with phosphorothioate
  source 1..60: mol_type = other DNA
tttttttttttttttttttttttttttttttttttttttttttttttttttttttttttt

SEQ ID NO: 7 - DNA, 70 bp, synthetic construct
  misc_feature 1..70: note = Synthetic polynucleotide
  misc_feature 1..3: note = Backbone modified with phosphorothioate
  misc_feature 67..69: note = Backbone modified with phosphorothioate
  source 1..70: mol_type = other DNA
aaaaaaaaaaaaaaaaaaaaaaaaaaaaaaaaaaaaaaaaaaaaaaaaaaaaaaaaaaaaaaaaaaaaaa

SEQ ID NO: 8 - DNA, 70 bp, synthetic construct
  misc_feature 1..70: note = Synthetic polynucleotide
  misc_feature 1..3: note = Backbone modified with phosphorothioate
  misc_feature 67..69: note = Backbone modified with phosphorothioate
  source 1..70: mol_type = other DNA
tttttttttttttttttttttttttttttttttttttttttttttttttttttttttttttttttttttt

SEQ ID NO: 9 - DNA, 80 bp, synthetic construct
  misc_feature 1..80: note = Synthetic polynucleotide
  misc_feature 1..3: note = Backbone modified with phosphorothioate
  misc_feature 77..79: note = Backbone modified with phosphorothioate
  source 1..80: mol_type = other DNA
aaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 10 - DNA, 80 bp, synthetic construct
  misc_feature 1..80: note = Synthetic polynucleotide
  misc_feature 1..3: note = Backbone modified with phosphorothioate
  misc_feature 77..79: note = Backbone modified with phosphorothioate
  source 1..80: mol_type = other DNA
tttttttttttttttttttttttttttttttttttttttttttttttttttttttttttttttttttttttttttttttt

SEQ ID NO: 11 - DNA, 90 bp, synthetic construct
  misc_feature 1..90: note = Synthetic polynucleotide
  misc_feature 1..3: note = Backbone modified with phosphorothioate
  misc_feature 87..89: note = Backbone modified with phosphorothioate
  source 1..90: mol_type = other DNA
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 12 - DNA, 90 bp, synthetic construct
  misc_feature 1..90: note = Synthetic polynucleotide
  misc_feature 1..3: note = Backbone modified with phosphorothioate
  misc_feature 87..89: note = Backbone modified with phosphorothioate
  source 1..90: mol_type = other DNA
tttttttttttttttttttttttttttttttttttttttttttttttttttttttttttttttttttttttttttttttttttttttttt

SEQ ID NO: 13 - DNA, 100 bp, synthetic construct
  misc_feature 1..100: note = Synthetic polynucleotide
  misc_feature 1..3: note = Backbone modified with phosphorothioate
  misc_feature 97..99: note = Backbone modified with phosphorothioate
  source 1..100: mol_type = other DNA
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 14 - DNA, 100 bp, synthetic construct
  misc_feature 1..100: note = Synthetic polynucleotide
  misc_feature 1..3: note = Backbone modified with phosphorothioate
  misc_feature 97..99: note = Backbone modified with phosphorothioate
  source 1..100: mol_type = other DNA
tttttttttttttttttttttttttttttttttttttttttttttttttttttttttttttttttttttttttttttttttttttttttttttttttttt

SEQ ID NO: 15 - DNA, 110 bp, synthetic construct
  misc_feature 1..110: note = Synthetic polynucleotide
  misc_feature 1..3: note = Backbone modified with phosphorothioate
  misc_feature 107..109: note = Backbone modified with phosphorothioate
  source 1..110: mol_type = other DNA
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 16 - DNA, 110 bp, synthetic construct
  misc_feature 1..110: note = Synthetic polynucleotide
  misc_feature 1..3: note = Backbone modified with phosphorothioate
  misc_feature 107..109: note = Backbone modified with phosphorothioate
  source 1..110: mol_type = other DNA
tttttttttttttttttttttttttttttttttttttttttttttttttttttttttttttttttttttttttttttttttttttttttttttttttttttttttttttt

SEQ ID NO: 17 - DNA, 30 bp, synthetic construct
  misc_feature 1..30: note = Synthetic polynucleotide
  misc_feature 1..3: note = Backbone modified with phosphorothioate
  misc_feature 27..29: note = Backbone modified with phosphorothioate
  source 1..30: mol_type = other DNA
gcgcgcgcgcgcgcgcgcgcgcgcgcgcgc

SEQ ID NO: 18 - DNA, 30 bp, synthetic construct; identical to SEQ ID NO: 17
  misc_feature 1..30: note = Synthetic polynucleotide
  misc_feature 1..3: note = Backbone modified with phosphorothioate
  misc_feature 27..29: note = Backbone modified with phosphorothioate
  source 1..30: mol_type = other DNA

SEQ ID NO: 19 - DNA, 60 bp, synthetic construct
  misc_feature 1..60: note = Synthetic polynucleotide
  misc_feature 1..3: note = Backbone modified with phosphorothioate
  misc_feature 57..59: note = Backbone modified with phosphorothioate
  source 1..60: mol_type = other DNA
gcgcgcgcgcgcgcgcgcgcgcgcgcgcgcgcgcgcgcgcgcgcgcgcgcgcgcgcgcgc

SEQ ID NO: 20 - DNA, 70 bp, synthetic construct
  misc_feature 1..70: note = Synthetic polynucleotide
  misc_feature 1..3: note = Backbone modified with phosphorothioate
  misc_feature 67..69: note = Backbone modified with phosphorothioate
  source 1..70: mol_type = other DNA
gcgcgcgcgcgcgcgcgcgcgcgcgcgcgcgcgcgcgcgcgcgcgcgcgcgcgcgcgcgcgcgcgcgcgc

SEQ ID NO: 21 - DNA, 80 bp, synthetic construct
  misc_feature 1..80: note = Synthetic polynucleotide
  misc_feature 1..3: note = Backbone modified with phosphorothioate
  misc_feature 77..79: note = Backbone modified with phosphorothioate
  source 1..80: mol_type = other DNA
gcgcgcgcgcgcgcgcgcgcgcgcgcgcgcgcgcgcgcgcgcgcgcgcgcgcgcgcgcgcgcgcgcgcgcgcgcgcgcgc

SEQ ID NO: 22 - DNA, 90 bp, synthetic construct
  misc_feature 1..90: note = Synthetic polynucleotide
  misc_feature 1..3: note = Backbone modified with phosphorothioate
  misc_feature 87..89: note = Backbone modified with phosphorothioate
  source 1..90: mol_type = other DNA
gcgcgcgcgcgcgcgcgcgcgcgcgcgcgcgcgcgcgcgcgcgcgcgcgcgcgcgcgcgcgcgcgcgcgcgcgcgcgcgcgcgcgcgcgc

SEQ ID NO: 23 - DNA, 100 bp, synthetic construct
  misc_feature 1..100: note = Synthetic polynucleotide
  misc_feature 1..3: note = Backbone modified with phosphorothioate
  misc_feature 97..99: note = Backbone modified with phosphorothioate
  source 1..100: mol_type = other DNA
gcgcgcgcgcgcgcgcgcgcgcgcgcgcgcgcgcgcgcgcgcgcgcgcgcgcgcgcgcgcgcgcgcgcgcgcgcgcgcgcgcgcgcgcgcgcgcgcgcgc

SEQ ID NO: 24 - DNA, 76 bp, synthetic construct
  misc_feature 1..76: note = Synthetic polynucleotide
  misc_feature 1..3: note = Backbone modified with phosphorothioate
  misc_feature 73..75: note = Backbone modified with phosphorothioate
  source 1..76: mol_type = other DNA
aaaaaaaaaaaaaaaaaaaaaaaaaaaaaaaaaaaaaaaaaaaaaaaaaaaaaaaaaaaaaaaaaaaaaaaaaaaa

SEQ ID NO: 25 - DNA, 76 bp, synthetic construct
  misc_feature 1..76: note = Synthetic polynucleotide
  misc_feature 1..3: note = Backbone modified with phosphorothioate
  misc_feature 73..75: note = Backbone modified with phosphorothioate
  source 1..76: mol_type = other DNA
tttttttttttttttttttttttttttttttttttttttttttttttttttttttttttttttttttttttttttt

SEQ ID NO: 26 - DNA, 76 bp, synthetic construct
  misc_feature 1..76: note = Synthetic polynucleotide
  misc_feature 1..3: note = Backbone modified with phosphorothioate
  misc_feature 73..75: note = Backbone modified with phosphorothioate
  source 1..76: mol_type = other DNA
acacacacacacacacacacacacacacacacacacacacacacacacacacacacacacacacacacacacacac

SEQ ID NO: 27 - DNA, 76 bp, synthetic construct
  misc_feature 1..76: note = Synthetic polynucleotide
  misc_feature 1..3: note = Backbone modified with phosphorothioate
  misc_feature 73..75: note = Backbone modified with phosphorothioate
  source 1..76: mol_type = other DNA
gtgtgtgtgtgtgtgtgtgtgtgtgtgtgtgtgtgtgtgtgtgtgtgtgtgtgtgtgtgtgtgtgtgtgtgtgtgt

SEQ ID NO: 28 - DNA, 76 bp, synthetic construct
  misc_feature 1..76: note = Synthetic polynucleotide
  misc_feature 1..3: note = Backbone modified with phosphorothioate
  misc_feature 73..75: note = Backbone modified with phosphorothioate
  source 1..76: mol_type = other DNA
atatatatatatatatatatatatatatatatatatatatatatatatatatatatatatatatatatatatatat

SEQ ID NO: 29 - DNA, 76 bp, synthetic construct
  misc_feature 1..76: note = Synthetic polynucleotide
  misc_feature 1..3: note = Backbone modified with phosphorothioate
  misc_feature 73..75: note = Backbone modified with phosphorothioate
  source 1..76: mol_type = other DNA
tatatatatatatatatatatatatatatatatatatatatatatatatatatatatatatatatatatatatata

SEQ ID NO: 30 - DNA, 76 bp, synthetic construct
  misc_feature 1..76: note = Synthetic polynucleotide
  misc_feature 1..3: note = Backbone modified with phosphorothioate
  misc_feature 73..75: note = Backbone modified with phosphorothioate
  source 1..76: mol_type = other DNA
actgactgactgactgactgactgactgactgactgactgactgactgactgactgactgactgactgactgactg

SEQ ID NO: 31 - DNA, 76 bp, synthetic construct
  misc_feature 1..76: note = Synthetic polynucleotide
  misc_feature 1..3: note = Backbone modified with phosphorothioate
  misc_feature 73..75: note = Backbone modified with phosphorothioate
  source 1..76: mol_type = other DNA
cagtcagtcagtcagtcagtcagtcagtcagtcagtcagtcagtcagtcagtcagtcagtcagtcagtcagtcagt

SEQ ID NO: 32 - DNA, 76 bp, synthetic construct
  misc_feature 1..76: note = Synthetic polynucleotide
  misc_feature 1..3: note = Backbone modified with phosphorothioate
  misc_feature 73..75: note = Backbone modified with phosphorothioate
  source 1..76: mol_type = other DNA
gaccctatcgatacagggcacggggtcgaactgttgggtttcgccatggtaccccctgcatttatatagccagacc

SEQ ID NO: 33 - DNA, 76 bp, synthetic construct
  misc_feature 1..76: note = Synthetic polynucleotide
  misc_feature 1..3: note = Backbone modified with phosphorothioate
  misc_feature 73..75: note = Backbone modified with phosphorothioate
  source 1..76: mol_type = other DNA
ggtctggctatataaatgcagggggtaccatggcgaaacccaacagttcgaccccgtgccctgtatcgatagggtc

SEQ ID NO: 34 - protein, 8 aa, synthetic construct
  REGION 1..8: note = Synthetic polypeptide
SIINFEKL

SEQ ID NO: 35 - DNA, 90 bp, synthetic construct; identical to SEQ ID NO: 11
  misc_feature 1..90: note = Synthetic polynucleotide
  misc_feature 1: note = Residue modified with FAM
  misc_feature 1..3: note = Backbone modified with phosphorothioate
  misc_feature 87..89: note = Backbone modified with phosphorothioate
  source 1..90: mol_type = other DNA

SEQ ID NO: 36 - DNA, 90 bp, synthetic construct; identical to SEQ ID NO: 12
  misc_feature 1..90: note = Synthetic polynucleotide
  misc_feature 1: note = Residue modified with FAM
  misc_feature 1..3: note = Backbone modified with phosphorothioate
  misc_feature 87..89: note = Backbone modified with phosphorothioate
  source 1..90: mol_type = other DNA

SEQ ID NO: 37 - DNA, 76 bp, synthetic construct
  misc_feature 1..76: note = Synthetic polynucleotide
  misc_feature 1..3: note = Backbone modified with phosphorothioate
  misc_feature 73..75: note = Backbone modified with phosphorothioate
  source 1..76: mol_type = other DNA
attaattaattaattaattaattaattaattaattaattaattaattaattaattaattaattaattaattaatta

SEQ ID NO: 38 - DNA, 76 bp, synthetic construct
  misc_feature 1..76: note = Synthetic polynucleotide
  misc_feature 1..3: note = Backbone modified with phosphorothioate
  misc_feature 73..75: note = Backbone modified with phosphorothioate
  source 1..76: mol_type = other DNA
taattaattaattaattaattaattaattaattaattaattaattaattaattaattaattaattaattaattaat

SEQ ID NO: 39 - DNA, 76 bp, synthetic construct; identical to SEQ ID NO: 30
  misc_feature 1..76: note = Synthetic polynucleotide
  misc_feature 1..3: note = Backbone modified with phosphorothioate
  misc_feature 73..75: note = Backbone modified with phosphorothioate
  source 1..76: mol_type = other DNA

SEQ ID NO: 40 - DNA, 76 bp, synthetic construct; identical to SEQ ID NO: 31
  misc_feature 1..76: note = Synthetic polynucleotide
  misc_feature 1..3: note = Backbone modified with phosphorothioate
  misc_feature 73..75: note = Backbone modified with phosphorothioate
  source 1..76: mol_type = other DNA

SEQ ID NO: 41 - DNA, 76 bp, synthetic construct; identical to SEQ ID NO: 32
  misc_feature 1..76: note = Synthetic polynucleotide
  misc_feature 1..3: note = Backbone modified with phosphorothioate
  misc_feature 73..75: note = Backbone modified with phosphorothioate
  source 1..76: mol_type = other DNA

SEQ ID NO: 42 - DNA, 76 bp, synthetic construct; identical to SEQ ID NO: 33
  misc_feature 1..76: note = Synthetic polynucleotide
  misc_feature 1..3: note = Backbone modified with phosphorothioate
  misc_feature 73..75: note = Backbone modified with phosphorothioate
  source 1..76: mol_type = other DNA

SEQ ID NO: 43 - DNA, 90 bp, synthetic construct; identical to SEQ ID NO: 11
  misc_feature 1..90: note = Synthetic polynucleotide
  misc_feature 1..3: note = Backbone modified with phosphorothioate
  misc_feature 87..89: note = Backbone modified with phosphorothioate
  source 1..90: mol_type = other DNA

SEQ ID NO: 44 - DNA, 90 bp, synthetic construct; identical to SEQ ID NO: 12
  misc_feature 1..90: note = Synthetic polynucleotide
  misc_feature 1..3: note = Backbone modified with phosphorothioate
  misc_feature 87..89: note = Backbone modified with phosphorothioate
  source 1..90: mol_type = other DNA

SEQ ID NO: 45 - DNA, 90 bp, synthetic construct; identical to SEQ ID NO: 22
  misc_feature 1..90: note = Synthetic polynucleotide
  misc_feature 1..3: note = Backbone modified with phosphorothioate
  misc_feature 87..89: note = Backbone modified with phosphorothioate
  source 1..90: mol_type = other DNA

SEQ ID NO: 46 - DNA, 90 bp, synthetic construct
  misc_feature 1..90: note = Synthetic polynucleotide
  misc_feature 1..3: note = Backbone modified with phosphorothioate
  misc_feature 87..89: note = Backbone modified with phosphorothioate
  source 1..90: mol_type = other DNA
cgcgcgcgcgcgcgcgcgcgcgcgcgcgcgcgcgcgcgcgcgcgcgcgcgcgcgcgcgcgcgcgcgcgcgcgcgcgcgcgcgcgcgcgcg

(19) United States
(12) Patent Application Publication
(10) Pub. No.: US 2023/0241092 A1
(43) Pub. Date: Aug. 3, 2023

(54) LIPID NANOPARTICLES FOR DELIVERY OF STING-DEPENDENT ADJUVANTS

(71) Applicant: University of Maimi, Maimi, FL (US)

(72) Inventor: GLEN N BARBER, Miami, FL (US)

(73) Assignee: UNIVERSITY OF MIAMI, Miami, FL (US)

(21) Appl. No.: 18/176,406

(22) Filed: Feb. 28, 2023

Related U.S. Application Data
(63) Continuation-in-part of application No. 16/621,820, filed on Dec. 12, 2019, now Pat. No. 11,629,346.
Application No. 16/621,820 is a continuation-in-part of application No. PCT/US22/34796, filed on Jun. 23, 2022.
(60) Provisional application No. 62/518,292, filed on Jun. 12, 2017.
(60) Provisional application No. 63/214,671, filed on Jun. 24, 2021.
(60) Provisional application No. 63/349,004, filed on Jun. 3, 2022.
(60) Provisional application No. 63/354,199, filed on Jun. 21, 2022.

(51) Int. Cl. A61K 31/711; A61K 9/51; A61K 39/00; A61P 35/00

(52) U.S. Cl. CPC A61K 31/711; A61K 9/5123; A61K 39/4615; A61P 35/00

(57) ABSTRACT

Activation of STimulator of INterferon Genes (STING) triggers cytokine production and facilitates tumor antigen cross-presentation. In an embodiment of the present invention, STING-dependent innate immune signaling pathway activators (STAVs) can be delivered to antigen presenting cells. In various embodiments of the present invention, the STAVs can be delivered in lipid nanoparticle formulations. In various embodiments of the present invention, the range of cancers amenable to STAV therapy can be extended using a non-cell-based nanoparticle strategy that effectively delivers Nano-STAVs into the Tumor Micro Environment (TME) to potently generate anti-tumor cytotoxic T cell activity. The STAV formulations can be introduced into solid tumors present in the mammal. Alternatively, the Nano-STAVs can be introduced through direct inoculation. The lipid nanoparticles stick to the tumor cells and are co-phagocytosed to activate STING in APC's.

### PRIORITY CLAIM

This application claims priority to and is a continuation in part of (1) U.S. application Ser. No. 16/621,820 filed Dec. 12, 2019, entitled STING-DEPENDENT ACTIVATORS FOR TREATMENT OF DISEASE, by Glen N. Barber which is the national phase of (2) PCT Application No. PCT/US2018/036997 entitled “STING-Dependent Activators for Treatment of Disease”, inventor Glen N. Barber, filed Jun. 12, 2018, which claims priority to (3) U.S. Provisional Patent Application No. 62/518,292 filed Jun. 12, 2017 and this application also claims priority to and is a continuation in part of (4) PCT Application No. PCT/US22/034796 entitled “Lipid nanoparticle vector for delivery of Sting Dependent Adjuvants (STAVS)”, inventor Glen N. Barber, filed Jun. 23, 2022, which claims priority to (5) U.S. Provisional Patent Application No. 63/214,671, filed Jun. 24, 2021 and (6) U.S. Provisional Patent Application No. 63/349,004, filed Jun. 24, 2022, and (7) U.S. Provisional Patent Application No. 63/354,199, filed Jun. 21, 2022 which applications (1)-(7) are herein incorporated by reference in their entireties and for all purposes.

The Sequence Listing in WIPO Standard ST.26 form written in file STNG-01014US0_ST26.xml, created Feb. 28, 2023, 99, 119 bytes, machine format IBM-PC, MS-Windows operating system, is hereby incorporated by reference in its entirety and for all purposes.

### FIELD OF THE INVENTION

Embodiments of the invention relate to compositions and methods for modulating innate and adaptive immunity in a subject and/or for the treatment of an immune-related disorder, cancer, autoimmunity, treating and preventing infections with Sting Dependent Adjuvants.

### BACKGROUND OF THE INVENTION

Cellular host defense responses to pathogen invasion principally involves the detection of pathogen associated molecular patterns (PAMPs) such as viral nucleic acid or bacterial cell wall components including lipopolysaccharide or flagellar proteins that results in the induction of anti-pathogen genes. For example, viral Ribonucleic Acid (RNA) can be detected by membrane bound Toll-like receptors (TLR's) present in the Endoplasmic Reticulum (ER) and/or endosomes (e.g. Toll-like receptor 3 (TLR 3) and TLR7/TLR8) or by TLR-independent intracellular DExD/H box RNA helicases referred to as Retinoic acid Inducible Gene 1 (RIG-1) or Melanoma Differentiation associated Antigen 5 (MDA5), also referred to as IFIH1 and helicard. These events culminate in the activation of downstream signaling events, much of which remains unknown, leading to the transcription of Nuclear Factor kappa-light-chain-enhancer of activated B cells (NF-κB) and Interferon Regulatory Factor 3 (IRF3)/IRF7-dependent genes, including type I Interferon (IFN).

ATLL was first described as a distinct clinical entity in 1979 and its association with the human T-cell leukemia virus type 1 (HTLV-1) was reported shortly thereafter. HTLV-1 affects about 10-20 million people worldwide and is endemic in southwest Japan, sub-Saharan Africa, the Caribbean, and parts of South America, particularly Brazil and Peru. South Florida (containing Miami and Broward counties) due to its close proximity to the Caribbean, has a large population of immigrants from HTLV-1 endemic areas, therefore ATLL is commonly encountered in this geographic area. African-American patients are also frequently diagnosed with ATLL at the University of Miami and Jackson Memorial Hospital. ATLL patients are also frequently encountered in New York City. ATLL can present in multiple forms and is generally sub-classified into four subtypes. Lymphoma and acute ATLL are the two most aggressive variants where patients usually present with a high tumor burden and hypercalcemia. The chronic and smoldering forms of ATLL have a more indolent course, although they often progress to the more malignant forms of the disease. ATLL carries a dismal prognosis, and is generally incurable with conventional chemotherapy alone. In a Japanese study of 1,594 patients with ATLL treated with modern aggressive therapies between 2000 and 2009, the median survival (MS) times were 8.3 and 10.6 months for acute and lymphomatous types respectively. A subset of patients with leukemic types of ATLL with non-bulky tumors or lymph nodes may benefit long-term from AZT-interferon-α therapy, however, this treatment is only suppressive and all patients ultimately relapse and succumb to their disease. A relatively small group of patients become eligible for allogeneic stem cell transplant (allo-HSCT) with the possibility of long-term cure. Despite this, in the Katsuya study, 227 patients who underwent allo-HSCT were included, and the MS was only 5.9 months with 4-yr survival of 26%.

AML is the most common form of acute leukemia in adults and accounts for the largest number of deaths from leukemias in the United States. Over 20,000 people are diagnosed with AML per year, and roughly half of this number die of it each year. AML usually affects older patients with the median age at diagnosis at 67 years. Standard induction chemotherapy regimens are used for patients younger than age 60 consisting of a backbone of cytarabine plus an anthracycline. Complete response rates for patients who are 50 years or younger are in the range of 60% to 70% but most patients ultimately relapse and succumb to their disease. Poor performance status and medical co-morbidities limit the ability to administer aggressive standard therapy to older patients and those that do get treated often receive suboptimal treatment. In elderly patients treated with intensive chemotherapy there has been little improvement in survival indicating the need for alternative approaches. Currently, the 5-year overall survival for AML hovers around 25%, while in patients 65 or older it is <10%.

B-cell or T-cell ALL is the least common type of acute leukemia in adults although it is the most common in the pediatric population. Adult patients have a relatively poor prognosis as compared to children and young adults, who can be cured with intensive chemotherapy. Prognosis varies according to disease presentation and molecular subtypes. For instance, the 5-year overall survival rate among adult patients with Philadelphia chromosome-positive (Ph+) pre-B-cell ALL is only 25%. The relapsed disease setting is often fatal in adults.

STimulator of Interferon Genes (STING) is a 379 amino acid transmembrane protein located in the cytosol endoplasmic reticulum of for example fibroblasts, macrophages and DCs. STING is a DNA sensor that has evolved to detect microbial infection of the cell. STING is activated by cyclic dinucleotides (CDN's) such as cyclic di-GMP and cyclic-di-AMP secreted by intracellular bacteria following infection. Alternatively, STING can be activated by cyclic GMP-AMP (cGAMP) generated by a cellular cGAMP synthase cGAS (MB21D1) after association with aberrant cytosolic dsDNA species, which can include microbial DNA or self-DNA leaked from the nucleus into the cytosol. CDN-binding results in STING, complexed with the IRF3 kinase TANK-binding kinase 1 (TBK1) re-locating to perinuclear regions of the cell. Association with CDN's enables STING to activate the transcription factors IRF3 and NF-κB which stimulate the production of type I interferon (IFN) and pro-inflammatory cytokines, which facilitate adaptive immunity.

Lipid nanoparticle technology offers the promise of high nucleic acid encapsulation efficiency, potent transfection and improved penetration of Sting Dependent Adjuvants (STAVs) into cells with low cytotoxicity and immunogenicity.

Programmed death-ligand 1 (PD-L1) together with its receptor programmed cell death protein 1 (PD-1) are ‘check point’ proteins involved in the regulation of the immune response. The interaction of these cell surface proteins can for example suppress the immune system following infection to limit the killing of bystander host cells. These checkpoint proteins can be used by some types of cancer to block the immune system's ability to attack the cancerous cells.

PD-L1 inhibitors and PD-1 inhibitors are check point inhibitors of PD-L1 and PD-1 respectively and act to inhibit the association of the PD-L1 with PD-1. By blocking the activity of PD-L1 and PD-1 the inhibitors can be used to restore the immune system's ability to attack the cancerous cells and therefore used as anticancer drugs.

One in five Americans will develop skin cancer by the age of seventy. It is the most common of all solid tumors. One of the most serious skin cancers is melanoma, where an estimated 200,000 people will be diagnosed in the USA in 2021. A variety of treatments are available, including vaccines, kinase inhibitors as well as oncolytic viruses (T-VEC; Imlygic, talimogene laherparevec) with varying response rates. In 2015, the FDA approved T-VEC for patients with advanced melanoma (Stage IIIB, IIIC or IV) that could not be removed with surgery and were refractory to alternate treatments. T-VEC is injected cutaneous, sub-cutaneous or directly into nodes.

### SUMMARY OF THE INVENTION

Tumor cells are notoriously non-immunogenic through their ability to mimic the properties of normal cells which have naturally evolved to avoid activating the immune system following cell death and phagocytosis. In an embodiment of the present invention, a new approach overcomes this obstacle and makes previously immuno-evasive, inert tumor cells highly immunogenic. This has been achieved by developing DNAse-resistant nucleic acid-based STING-dependent adjuvants or activators, referred to as STAVs (dsDNA species of approximate length 76 nucleotides) as activators of the STING-dependent innate immune signaling pathway. In an embodiment of the present invention, syngeneic tumor cells loaded with STAVs rendered non-immunogenic cells immunogenic. In an embodiment of the present invention, the syngeneic tumor cells loaded with STAVs are able to stimulate antigen presenting cells (APCs) in vitro and in vivo. Immunocompetent mice bearing metastatic, melanoma tumors could be cured following inoculation of syngeneic tumor cells loaded with STAVs. In an embodiment of the present invention, syngeneic tumor cells loaded with STAVs ex vivo can be used to treat autologous aggressive leukemia cells (ATLL, AML, and ALL) concomitant with a personalized dendritic cell (DCs) vaccine (prepared from DCs stimulated by dead UV-irradiated STAVs loaded leukemic cells).

DCs are specialized APCs found in blood and throughout most organ tissues. DCs strongly express major histocompatibility complex (MHC), adhesion, and co-stimulatory molecules necessary for the stimulation of T cell responses and adaptive cell immunity. DCs are located at sites of antigen capture and after they phagocyte pathogens, foreign antigens, or damaged cells they subsequently migrate to lymphatic areas for antigen presentation. By expressing both MHC class I and class II molecules, they can prime both cytotoxic CD8+ cells and CD4+ helper T-cells respectively, and both of these cell types are thought to be necessary for an effective cell-mediated immune response. DCs can also strongly activate NK and NK-T cells thus linking innate and adaptive immune responses thus potentially targeting tumor cells for killing with and without expression of MHC class I molecules. DCs have been demonstrated to interact with foreign antigens ex vivo, present these to naïve CD4C T cells, and to generate clonal expansion of effector T cells.

DC vaccines have emerged as promising cancer immunotherapy approach. DC vaccines can be generated from large numbers of progenitor cells cultured ex vivo in the presence of cytokines after exposing these to foreign antigens. Tumor cells can evade immune recognition by blunting T cell responses via several mechanisms; these may include: 1) presenting tumor antigens in the relative absence of co-stimulatory molecules required for the activation of effector T cells thus inducing T cell anergy rather than immunity, 2) creating a micro-environment rich in immunosuppressive T-regulatory cells (Tregs) and myeloid derived suppressor cells, and 3) upregulating negative co-stimulatory pathways such as those mediated by CTLA-4 and PDL-1/PD-1 thus favoring tumor growth and survival. Malignant cells can also inhibit the function of DCs thus making them more tolerant to tumor antigens. In an embodiment of the present invention, an effective cancer vaccine requires efficient presentation of tumor antigens, adequate co-stimulation leading to T-cell priming, and successful reversal of the immunosuppression induced by tumor cells in order to achieve long-term immunity. Animal models have demonstrated that DC tumor vaccines can reverse T-cell anergy resulting in subsequent tumor rejection.

Clinical trials and pre-clinical studies have evaluated DC vaccines against various cancers, including hematologic malignancies, and demonstrated safety. In one study, a personalized whole tumor cell (AML)/DC fusion vaccine elicited the expansion of leukemia-specific T cells and protected against disease relapse in elderly patients with AML. A recently developed HTLV-1 Tax-DC vaccine consisting of autologous DCs pulsed with Tax peptides corresponding to CTL epitopes was administered to three pre-treated ATLL patients, and two patients survived for more than 4 years after vaccination without severe adverse effects. DCs loaded with leukemia-derived apoptotic bodies from adult patients with ALL increased their ability to stimulate both allogeneic and autologous T lymphocytes, and to generate specific anti-leukemic CD3+ cells. These findings offered a rationale for designing DC-based vaccines for patients with ALL with the objective of controlling/eradicating the disease. In an embodiment of the present invention, the paradigm will include personalized serial injections of autologous mature DCs stimulated exogenously with patient's own STAVs loaded leukemic cells.

ATLL is a clonal disease triggered by HTLV-1 infection that is invariably lethal and for which there is no cure or vaccine. Relapsed/refractory AML and ALL in adult patients are also incurable and often rapidly fatal despite aggressive treatment. Recent clinical trials testing the use of adjuvant vaccination with antigen stimulated autologous mature DCs have shown that DC vaccination is safe, feasible, and potentially beneficial for patients. The stimulation of innate immune signaling pathways leading to cytokine production within phagocytes such as CD8+ DCs involve STING. In an embodiment of the present invention, a new generation of innate immune activators that trigger STING signaling are referred to as STAVs: STING dependent adjuvants or activators. Tumor cells transfected with STAVs activate APCs in trans and generate potent anti-tumor T cell activity. The ability of dying cells to activate APCs is carefully controlled to avoid unwarranted inflammatory responses. Thus, dying tumor cells avoid aggravating APCs which following phagocytosis do not trigger inflammatory responses required for efficient CTL priming. However, dying tumor cells containing exogenous innate immune agonists such as cytosolic DNA, escape anti-inflammatory defenses and potently activate APCs in trans through extrinsic innate immune, STING-dependent signaling to generate potent CTL activity. In the absence of STING agonists, dying cells are ineffectual in the stimulation of APCs in trans. Indeed, cytosolic STING activators, including cytosolic DNA and cyclic dinucleotides (CDNs), constitute cellular danger associated molecular patterns (DAMPs) (usually only generated by viral infection or following DNA-damage events) that can render tumor cells highly immunogenic.

STING, a molecule that plays a key role in the innate immune response, includes 5 putative transmembrane (TM) regions, predominantly resides in the endoplasmic reticulum (ER), and is able to activate both NF-κs and IRF3 transcription pathways to induce type I IFN and to exert a potent anti-viral state following expression (see U.S. patent application Ser. No. 16/717,325 and PCT/US2009/052767 each of which is incorporated herein by reference in its entirety and for all purposes). Loss of STING reduced the ability of Polyinosinic:polycytidylic acid (polyIC) to activate type I IFN and rendered Murine Embryonic Fibroblasts (MEFs) lacking STING (^{-/-} MEFs) generated by targeted homologous recombination, susceptible to vesicular stomatitis virus (VSV) infection. In the absence of STING, DNA-mediated type I IFN responses were inhibited, indicating that STING may play an important role in recognizing DNA from viruses, bacteria, and other pathogens which can infect cells. Yeast-two hybrid and co-immunoprecipitation studies indicated that STING interacts with RIG-1 and with Ssr2/TRAPβ, a member of the translocon-associated protein (TRAP) complex required for protein translocation across the ER membrane following translation. RNAi ablation of TRAPP inhibited STING function and impeded the production of type I IFN in response to polyIC.

Further experiments showed that STING itself binds nucleic acids including single- and double-stranded DNA such as from pathogens and apoptotic DNA, and plays a central role in regulating pro-inflammatory gene expression in inflammatory conditions such as DNA-mediated arthritis and cancer. Various new methods of, and compositions for, upregulating STING expression or function are described herein along with further characterization of other cellular molecule which interact with STING. These discoveries allow for the design of new adjuvants, vaccines and therapies to regulate the immune system and other systems.

In one aspect, the present application relates to a composition for treating a human subject suffering from cancer comprising a Nano-STAV comprising a double-stranded DNA; and a lipid nanoparticle comprising a polymer-conjugated lipid, a sterol, a phospholipid; and an ionizing lipid.

In one aspect, the present application relates to a composition for treating a human subject suffering from cancer comprising a first Nano-STAV comprising a first STAV selected from the group consisting of STAV1=(SEQ ID NO:24)+(SEQ ID NO:25); STAV2=(SEQ ID NO:26)+(SEQ ID NO:27); and STAV3=(SEQ ID NO:37)+(SEQ ID NO:38), and a LNP comprising a polymer-conjugated lipid, a sterol, a phospholipid, and an ionizing lipid.

In one aspect, the present application relates to a composition for treating a human subject suffering from cancer comprising a first Nano-STAV comprising a first STAV selected from the group consisting of STAV1=(SEQ ID NO:24)+(SEQ ID NO:25); STAV2=(SEQ ID NO:26)+(SEQ ID NO:27); and STAV3=(SEQ ID NO:37)+(SEQ ID NO:38), and a LNP comprising a polymer-conjugated lipid selected from the group consisting of DMG-PEG 2000 and DSPE PEG 2000, cholesterol, distearoylphosphatidylcholine, and (6Z,9Z,28Z,31Z)-Heptatriaconta-6,9,28,31-tetraen-19-yl 4-(dimethylamino)butanoate.

In another aspect, the present application relates to a pharmaceutical composition comprising a therapeutically effective amount of the composition of the application and a pharmaceutically acceptable carrier.

In an embodiment of the present invention, a method of modulating (e.g., inhibiting or stimulating) a STING protein involves application of the composition of the application or the pharmaceutical composition. The method comprises administering to a subject in need thereof an effective amount of the composition of the application or a pharmaceutically acceptable salt or ester thereof, or a pharmaceutical composition of the application. In one embodiment, the STING protein is a human STING protein.

In an embodiment of the present invention, a method of treating or preventing a disease, wherein the disease is caused by, or associated with, STING expression, activity, and/or function (e.g., deregulation of STING expression, activity, and/or function) involves application of the composition of the application. The method further comprises administering to a subject in need thereof an effective amount of the composition of the application or a pharmaceutically acceptable salt or ester thereof, or a pharmaceutical composition of the application.

In an embodiment of the present invention, a method of treating or preventing a disease associated with deregulation of one or more of the intracellular pathways in which a STING protein is involved (e.g., deregulation of intracellular dsDNA mediated type I interferon activation). The method comprises administering to a subject in need thereof an effective amount of the composition of the application or a pharmaceutically acceptable salt or ester thereof, or a pharmaceutical composition of the application.

In an embodiment of the present invention, a kit comprising the composition of the application or a pharmaceutically acceptable salt or ester thereof, or a pharmaceutical composition of the application.

In an embodiment of the present invention, a compound of the application or a pharmaceutically acceptable salt or ester thereof, or a pharmaceutical composition of the application, for use in the manufacture of a medicament for modulating (e.g., inhibiting or stimulating) a STING protein, for treating or preventing a disease, wherein the diseases is caused by, or associated with, STING expression, activity, and/or function (e.g., deregulation of STING expression, activity, and/or function), or for treating or preventing a disease associated with deregulation of one or more of the intracellular pathways in which a STING protein is involved (e.g., deregulation of intracellular dsDNA mediated type I interferon activation).

In an embodiment of the present invention, use of the composition of the application or a pharmaceutically acceptable salt or ester thereof, or a pharmaceutical composition of the application, in the manufacture of a medicament for modulating (e.g., inhibiting or stimulating) a STING protein, for treating or preventing a disease, wherein the diseases is caused by, or associated with, STING expression, activity, and/or function (e.g., deregulation of STING expression, activity, and/or function), or for treating or preventing a disease associated with deregulation of one or more of the intracellular pathways in which a STING protein is involved (e.g., deregulation of intracellular dsDNA mediated type I interferon activation).

In an embodiment of the present invention, the composition of the application or a pharmaceutically acceptable salt or ester thereof, or a pharmaceutical composition of the application, for use in modulating (e.g., inhibiting or stimulating) a STING protein, in treating or preventing a disease, wherein the diseases is caused by, or associated with, STING expression, activity, and/or function (e.g., deregulation of STING expression, activity, and/or function), or in treating or preventing a disease associated with deregulation of one or more of the intracellular pathways in which a STING protein is involved (e.g., deregulation of intracellular dsDNA mediated type I interferon activation).

In an embodiment of the present invention, use of the composition of the application or a pharmaceutically acceptable salt or ester thereof, or a pharmaceutical composition of the application, in modulating (e.g., inhibiting or stimulating) a STING protein, in treating or preventing a disease, wherein the diseases is caused by, or associated with, STING expression, activity, and/or function (e.g., deregulation of STING expression, activity, and/or function), or in treating or preventing a disease associated with deregulation of one or more of the intracellular pathways in which a STING protein is involved (e.g., deregulation of intracellular dsDNA mediated type-1 interferon activation).

The present application provides nano-STAVs that are therapeutic agents in the treatment or prevention of diseases such as cancer inflammation, and other immunological disorders.

The details of the disclosure are set forth in the accompanying description below. Although methods and materials similar or equivalent to those described herein can be used in the practice or testing of the present application, illustrative methods and materials are now described. In the case of conflict, the present specification, including definitions, will control. In addition, the materials, methods, and examples are illustrative only and are not intended to be limiting. Other features, objects, and advantages of the disclosure will be apparent from the description and from the claims. In the specification and the appended claims, the singular forms also include the plural unless the context clearly dictates otherwise. Unless defined otherwise, all technical and scientific terms used herein have the same meaning as commonly understood by one of ordinary skill in the art to which this disclosure belongs.

### BRIEF DESCRIPTION OF THE DRAWINGS

Various embodiments of the present invention will be described in detail based on the following Figures, where:

FIG. 1 is a line drawing representation showing the confocal analysis of B16 OVA cells transfected with no DNA, labeled with DAPI 210 and anti-calreticulin 205;

FIG. 2 is a line drawing representation of showing confocal analysis of B16 OVA cells transfected with STAVs-FAM, labeled with FAM 215 DAPI 210 and anti-calreticulin 205;

FIG. 3A shows flow cytometry analysis of B16 OVA cells transfected with no DNA;

FIG. 3B shows flow cytometry analysis of B16 OVA cells transfected with STAVs-FAM;

FIG. 4A shows a Transmission Electron Microscopy image of Nano-Empty LNPs at high magnification;

FIG. 4B shows a Transmission Electron Microscopy image of Nano-STAV LNPs at high magnification;

FIG. 4C shows a Transmission Electron Microscopy image of Nano-Empty LNPs at low magnification;

FIG. 4D shows a Transmission Electron Microscopy image of Nano-STAV LNPs at low magnification;

FIG. 4E shows a plot of cytokine expression for CXCL10 measured with qPCR in WT bone marrow derived mouse macrophages (11=PBS, 32=STAV, 33=STAV+lipofectamine, 34=Nano-Empty, and 35=Nano-STAV);

FIG. 4F shows a plot of cytokine expression for CXCL10 measured with qPCR in SKO bone marrow derived mouse macrophages (11=PBS, 32=STAV, 33=STAV+lipofectamine, 34=Nano-Empty, and 35=Nano-STAV);

FIG. 4G shows a plot of cytokine expression for CXCL10 measured with qPCR in MAVS KO bone marrow derived mouse macrophages (11=PBS, 32=STAV, 33=STAV+lipofectamine, 34=Nano-Empty, and 35=Nano-STAV), according to various embodiments of the present invention;

FIG. 4H shows a plot of cytokine expression for IFN3 measured with ELISA in WT bone marrow derived mouse macrophages (11=PBS, 32=STAV, 33=STAV+lipofectamine, 34=Nano-Empty, and 35=Nano-STAV), according to various embodiments of the present invention;

FIG. 4I shows a plot of cytokine expression for IFN3 measured with ELISA in SKO bone marrow derived mouse macrophages (11=PBS, 32=STAV, 33=STAV+lipofectamine, 34=Nano-Empty, and 35=Nano-STAV), according to various embodiments of the present invention;

FIG. 4J shows a plot of cytokine expression for IFN3 measured with ELISA in MAVS KO bone marrow derived mouse macrophages (11=PBS, 32=STAV, 33=STAV+lipofectamine, 34=Nano-Empty, and 35=Nano-STAV), according to various embodiments of the present invention;

FIG. 4K shows a plot of cytokine expression for CXCL10 measured with qPCR in DCs (11=PBS, 32=STAV, 33=STAV+lipofectamine, 34=Nano-Empty, and 35=Nano-STAV), according to various embodiments of the present invention;

FIG. 4L shows a plot of IFN3 measured with ELISA in DCs (11=PBS, 32=STAV, 33=STAV+lipofectamine, 34=Nano-Empty, and 35=Nano-STAV), according to various embodiments of the present invention;

FIG. 5A shows tumor volume of mice s.c. injected with B16 OVA cells (5×10⁵ cells/mouse) on the right flank. On day 7, 10, and 13 after tumor inoculation, the mice were i.t. injected with 11=PBS, 32=STAV, 34=Nano-Empty, and 35=Nano-STAV (0.1 μg/mouse). The tumor volume was measured and calculated with the formula V=(length×width²)/2. At 17 days, the spleen was extracted to measure IFNγ release from CD8+ T cells, according to various embodiments of the present invention;

FIG. 5B shows digital photographs of mice treated as in FIG. 5A, according to various embodiments of the present invention;

FIG. 5C shows IFNγ-ELISPOT of (OVA−) mice treated as in FIG. 5A;

FIG. 5D shows IFNγ-ELISPOT of (OVA+) mice (i.e., s.c. injected with B16 OVA cells (5×10⁵ cells/mouse) on the right flank) and treated as in FIG. 5A, according to various embodiments of the present invention;

FIG. 5E shows tumor volume of mice treated as in FIG. 5A or 36=HSV1-γ34.5;

FIG. 6A shows tumor volume of mice treated as in FIG. 4A with 11=PBS, 32=STAV, 37=anti-PD1 (50 μg/mice) administered i.p., 34=Nano-Empty, 38=Nano-Empty+anti-PD1 (50 μg/mice) administered i.p., 35=Nano-STAV (0.1 μg/mouse), and 39=Nano-STAV+anti-PD1 (50 μg/mice) administered i.p., according to various embodiments of the present invention;

FIG. 6B shows digital photographs of mice treated as in FIG. 6A, according to various embodiments of the present invention;

FIG. 6C shows IFNγ-ELISPOT of (OVA−) mice treated as in FIG. 6A;

FIG. 6D shows IFNγ-ELISPOT of (OVA+) mice (i.e., s.c. injected with B16 OVA cells (5×10⁵ cells/mouse) on the right flank) and treated as in FIG. 6A, according to an embodiment of the present invention;

FIG. 7A a flow diagram showing the protocol for administration of Nano-STAVs according to an embodiment of the present invention;

FIG. 7B a flow diagram showing the protocol for administration of Nano-STAVs according to an embodiment of the present invention;

FIG. 7C a flow diagram showing the protocol for administration of Nano-STAVs with check point inhibitors according to an embodiment of the present invention;

FIG. 7D a flow diagram showing the protocol for administration of Nano-STAVs with check point inhibitors according to an embodiment of the present invention;

FIG. 8A is a histogram showing an IFN3 ELISA assay in mouse embryonic fibroblasts (MEFs) Wild Type (WT) or STING Knock Out (SKO) cells transfected with different lengths of AT rich-STING ligands (lipofectamine 2000 transfection reagent only 11; A:T30ES 22 (SEQ ID NO:1, SEQ ID NO:2); A:T50ES 23 (SEQ ID NO:3, SEQ ID NO:4); A:T60ES 24 (SEQ ID NO:5, SEQ ID NO:6); A:T70ES 25 (SEQ ID NO:7, SEQ ID NO:8); A:T80ES 26 (SEQ ID NO:9, SEQ ID NO:10); A:T90ES (27 (SEQ ID NO:11, SEQ ID NO:12); and A:T100ES 28 (SEQ ID NO:13, SEQ ID NO:14));

FIG. 8B is a histogram showing an IFN3 ELISA assay in hTERT fibroblasts transfected with different lengths of AT rich-STING ligands (11; A:T30ES 22, A:T50ES 23, A:T60ES 24; A:T70ES 25; A:T80ES 26; A:T90ES 27; and A:T100ES 28);

FIG. 8C is a histogram showing a quantitative Real Time-Polymerase Chain Reaction (qRT-PCR) analysis of IFN31 in human macrophages transfected with different length of AT rich-STING ligands (11; A:T30ES 22; A:T50ES 23; A:T60ES 24; A:T70ES 25; A:T80ES 26; A:T90ES 27; A:T100ES 28); and A:T110ES 29 (SEQ ID NO:15, SEQ ID NO:16));

FIG. 8D is a histogram showing an IFN3 ELISA assay in MEFs WT or SKO cells transfected with different lengths of GC rich-STING ligands (11; GC30ES 32 (SEQ ID NO:17); GC50ES 33 (SEQ ID No:18); GC60ES 34 (SEQ ID NO:19); GC70ES 35 (SEQ ID NO:20); GC80ES 36 (SEQ ID NO:21); GC90ES 37 (SEQ ID NO:22); and GC100ES 38 (SEQ ID NO:23));

FIG. 8E is a histogram showing an IFNβ ELISA assay hTERT fibroblast transfected with different lengths of GC rich-STING ligands (11; GC30ES 32; GC50ES 33; GC60ES 34; GC70ES 35; GC80ES 36; GC90ES 37; and GC100ES 38);

FIG. 8F is a histogram showing a qRT-PCR analysis of IFNβ1 in human macrophages transfected with different length of GC rich-STING ligands (11; GC30ES 32; GC50ES 33; GC60ES 34; GC70ES 35; GC80ES 36; GC90ES 37; and GC100ES 38);

FIG. 9A shows the growth in tumor volumes from WT (n=7/group) mice s.c. injected with murine B16 melanoma cells (B16-OVA cells) on the flank and subsequently injected i.t. every 3 days with 10 μg of STAVs (STAV1=SEQ ID NO:24, SEQ ID NO:25; STAV2=SEQ ID NO:26, SEQ ID NO:27; STAV3=SEQ ID NO:28, SEQ ID NO:29; STAV4=, SEQ ID NO:30, SEQ ID NO:31; STAV5=(SEQ ID NO:32, SEQ ID NO:33) or Phosphate Buffered Saline (PBS) (as a control) and measured on the indicated days;

FIG. 9B shows the growth in tumor volumes from SKO (n=7/group) mice s.c. injected with murine B16 melanoma cells (B16-OVA cells) on the flank and subsequently injected i.t. every 3 days with 10 μg of STAVs or PBS (as control) and measured on the indicated days;

FIG. 9C is a histogram showing the frequency of OVA specific CD8+ T cells (using OVA257-264 (SIINFEKL) peptide, SEQ ID NO:34) in the spleen from: WT (n=4/group) mice injected with PBS as control 12, with STAV1-STAV5 44; SKO (n=4/group) mice injected with PBS as control 13, with STAV1 45;

FIG. 10A shows tumor volumes of mice treated as follows: on Day 0, C1498 cells were s.c. inoculated in wild type C57/BL6 mice, where the C1498 cells (AML tumor cells) were transfected with STAVs (3 μg/ml) for 3 hours and irradiated by UV (120 mJ/cm for 1 minute) and incubated for 24 hours. The mice were intraperitoneally (i.p.) injected with the irradiated C1498 cells with/without STAVs three times, STAV1 on Day 2, STAV2 on Day 5, and STAV3 on Day 10; and measured on the indicated days, where the tumor size from mice treated with C1498 cells with PBS 12, mice treated with C1498 cells without STAVs 45, and mice treated with C1498 containing STAVs 44 were measured;

FIG. 10B shows the tumor weight measured on Day 16 of the mice treated as per FIG. 3A;

FIG. 10C shows an indirect ELISA analysis of plates pre-coated with STAV1 at 0.1 μg/ml, where serum from mice treated with C1498 cells with PBS 12, mice treated with C1498 cells without STAVs 45, and mice treated with C1498 containing STAVs 44 was added to the ELISA plate wells. Anti-dsDNA (Abcam: ab27156) 14 was used as calibrator (standard curve), mice treated with control cells (not C1498 cells AML tumor) were used as a control 13.

FIG. 10D shows flow cytometry analysis of splenocytes isolated on Day 16 and stained with anti-CD19-Alexa Fluor 700, where splenocytes from mice treated with C1498 cells with PBS 12, mice treated with C1498 cells without STAVs 45, and mice treated with C1498 containing STAVs 44, mice treated with anti-dsDNA (Abcam: ab27156) 14 was used as calibrator (standard curve), and mice treated with control cells (not C1498 cells AML tumor) were used as a control 13;

FIG. 10E shows flow cytometry analysis of splenocytes isolated on Day 16 and stained with anti-CD3-FITC, and anti-CD45-Pacific Blue antibodies, where splenocytes from mice treated with C1498 cells with PBS 12, mice treated with C1498 cells without STAVs 45, and mice treated with C1498 containing STAVs 44, mice treated with anti-dsDNA (Abcam: ab27156) 14 was used as calibrator (standard curve), and mice treated with control cells (not C1498 cells AML tumor) were used as a control 13;

FIG. 10F shows flow cytometry analysis of splenocytes isolated on Day 16 and stained with anti-CD4-PE and anti-CD3-FITC antibodies, where splenocytes from mice treated with C1498 cells with PBS 12, mice treated with C1498 cells without STAVs 45, and mice treated with C1498 containing STAVs 44, mice treated with anti-dsDNA (Abcam: ab27156) 14 was used as calibrator (standard curve), and mice treated with control cells (not C1498 cells AML tumor) were used as a control 13;

FIG. 10G shows flow cytometry analysis of splenocytes isolated on Day 16 and stained with anti-CD8a-PercP anti-CD3-FITC antibodies, where splenocytes from mice treated with C1498 cells with PBS 12, mice treated with C1498 cells without STAVs 45, and mice treated with C1498 containing STAVs 44, mice treated with anti-dsDNA (Abcam: ab27156) 14 was used as calibrator (standard curve), and mice treated with control cells (not C1498 cells AML tumor) were used as a control 13;

FIG. 11A shows immunoblot panels revealing phosphorylation of STING (pSTING) and IRF-3 (pIRF3) 4 hours after transfecting interferon regulatory DNA (ISD) in AML and ATLL (ATLL-84c or JAE) relative to unphosphorylated forms of STING and IRF3, and pTBK, cGAS, and b-Actin (loading control);

FIG. 11B shows the presence of fluorescent FAM labelled STAVs in AML cells;

FIG. 11C shows the presence of fluorescent FAM labelled STAVs in ATLL cells;

FIG. 11D qRT-PCR analysis of CXC110 in human macrophages 16 hours after exposure to AML cells transfected with STAVs 46 or not (Mock 19 vs. UV irradiated only 41;

FIG. 11E qRT-PCR analysis of IFNB1 in human macrophages 16 hours after exposure to AML cells transfected with STAVs 46 or not (Mock 19 vs. UV irradiated only 41;

FIG. 11F qRT-PCR analysis of CXC110 in human macrophages 16 hours after exposure to ATLL (ATLL-84c or JAE) cells transfected with STAVs 47 or not (Mock 20 vs. UV irradiated only 42;

FIG. 11G qRT-PCR analysis of IFNB1 in human macrophages 16 hours after exposure to ATLL (ATLL-84c or JAE) cells transfected with STAVs 47 or not (Mock 20 vs. UV irradiated only 42;

FIG. 12A shows flow cytometry analysis of splenocytes isolated and stained with the fluorescently labeled anti-CD3-FITC antibodies from C57/BL56 mice sacrificed on day 25 (9 days after a first boost), where the splenocytes were isolated from the mice i.p. injected with PBS 13, EL4 cells 12, EL4 cells transfected with STAV1 47, EL4-cGAS cells 45, or EL4-cGAS cells transfected with STAV1 45 (the mice were i.p. injected 24 hours after the cells were transfected with STAV1 (3 μg/mL) for 3 hours and UV irradiated (120 mJ/cm for 1 minute) and boosted with the STAV1 after 16 days;

FIG. 12B shows flow cytometry analysis of splenocytes isolated and stained with the fluorescently labeled anti-CD4-PE antibodies from C57/BL56 mice sacrificed on day 25 (9 days after a first boost), where the splenocytes were isolated from the mice i.p. injected with PBS 13, EL4 cells 12, EL4 cells transfected with STAV1 47, EL4-cGAS cells 45, or EL4-cGAS cells transfected with STAV1 45 (the mice were i.p. injected 24 hours after the cells were transfected with STAV1 (3 μg/mL) for 3 hours and UV irradiated (120 mJ/cm for 1 minute) and boosted with the STAV1 after 16 days;

FIG. 12C shows flow cytometry analysis of splenocytes isolated and stained with the fluorescently labeled anti-CD8a-PercP antibodies from C57/BL56 mice sacrificed on day 25 (9 days after a first boost), where the splenocytes were isolated from the mice i.p. injected with PBS 13, EL4 cells 12, EL4 cells transfected with STAV1 47, EL4-cGAS cells 45, or EL4-cGAS cells transfected with STAV1 45 (the mice were i.p. injected 24 hours after the cells were transfected with STAV1 (3 μg/mL) for 3 hours and UV irradiated (120 mJ/cm for 1 minute) and boosted with the STAV1 after 16 days;

FIG. 12D shows flow cytometry analysis of splenocytes isolated and stained with the fluorescently labeled anti-CD45-Pacific Blue antibodies from C57/BL56 mice sacrificed on day 25 (9 days after a first boost), where the splenocytes were isolated from the mice i.p. injected with PBS 13, EL4 cells 12, EL4 cells transfected with STAV1 47, EL4-cGAS cells 45, or EL4-cGAS cells transfected with STAV1 45 (the mice were i.p. injected 24 hours after the cells were transfected with STAV1 (3 μg/mL) for 3 hours and UV irradiated (120 mJ/cm for 1 minute) and boosted with the STAV1 after 16 days;

FIG. 12E shows flow cytometry analysis of splenocytes isolated and stained with the fluorescently labeled anti-CD19-Alexa Fluor 700 antibodies from C57/BL56 mice sacrificed on day 25 (9 days after a first boost), where the splenocytes were isolated from the mice i.p. injected with PBS 13, EL4 cells 12, EL4 cells transfected with STAV1 47, EL4-cGAS cells 45, or EL4-cGAS cells transfected with STAV1 45 (the mice were i.p. injected 24 hours after the cells were transfected with STAV1 (3 μg/mL) for 3 hours and UV irradiated (120 mJ/cm for 1 minute) and boosted with STAV1 after 16 days;

FIG. 12F shows flow cytometry analysis of splenocytes isolated and stained with the fluorescently labeled anti-CD49b-PE/Cy7 antibodies from C57/BL56 mice sacrificed on day 25 (9 days after a first boost), where the splenocytes were isolated from the mice i.p. injected with PBS 13, EL4 cells 12, EL4 cells transfected with STAV1 47, EL4-cGAS cells 45, or EL4-cGAS cells transfected with STAV1 45 (the mice were i.p. injected 24 hours after the cells were transfected with STAV1 (3 μg/mL) for 3 hours and UV irradiated (120 mJ/cm for 1 minute) and boosted with the STAV1 after 16 days;

FIG. 12G shows flow cytometry analysis of splenocytes isolated and stained with the fluorescently labeled anti-CD11b-FITC antibodies from C57/BL56 mice sacrificed on day 25 (9 days after a first boost), where the splenocytes were isolated from the mice i.p. injected with PBS 13, EL4 cells 12, EL4 cells transfected with STAV1 47, EL4-cGAS cells 45, or EL4-cGAS cells transfected with STAV1 45 (the mice were i.p. injected 24 hours after the cells were transfected with STAV1 (3 μg/mL) for 3 hours and UV irradiated (120 mJ/cm for 1 minute) and boosted with the STAV1 after 16 days;

FIG. 13A shows flow cytometry analysis of splenocytes isolated and stained with the fluorescently labeled anti-CD3-FITC antibodies from C57/BL56 mice sacrificed on day 12, where the splenocytes were isolated from the mice i.p. injected with C1498 cells (24 hours after the cells were transfected with STAV1 (3 μg/mL) for 3 hours and UV irradiated (120 mJ/cm for 1 minute) 44, or the UV irradiated cells (only) 45, and the PBS control 47 and boosted with the STAV1 after 6 days;

FIG. 13B shows flow cytometry analysis of splenocytes isolated and stained with the fluorescently labeled anti-CD4-PE antibodies from C57/BL56 mice sacrificed on day 12, where the splenocytes were isolated from the mice i.p. injected with C1498 cells (24 hours after the cells were transfected with STAV1 (3 μg/mL) for 3 hours and UV irradiated (120 mJ/cm for 1 minute) 44, or the UV irradiated cells (only) 45, and the PBS control 47 and boosted with the STAV1 after 6 days;

FIG. 13C shows flow cytometry analysis of splenocytes isolated and stained with the fluorescently labeled anti-CD8-PercP antibodies from C57/BL56 mice sacrificed on day 12, where the splenocytes were isolated from the mice i.p. injected with C1498 cells (24 hours after the cells were transfected with STAV1 (3 μg/mL) for 3 hours and UV irradiated (120 mJ/cm for 1 minute) 44, or the UV irradiated cells (only) 45, and the PBS control 47 and boosted with the STAV1 after 6 days;

FIG. 13D shows flow cytometry analysis of splenocytes isolated and stained with the fluorescently labeled anti-CD45-Pacific Blue antibodies from C57/BL56 mice sacrificed on day 12, where the splenocytes were isolated from the mice i.p. injected with C1498 cells (24 hours after the cells were transfected with STAV1 (3 μg/mL) for 3 hours and UV irradiated (120 mJ/cm for 1 minute) 44, or the UV irradiated cells (only) 45, and the PBS control 47 and boosted with the STAV1 after 6 days;

FIG. 13E shows flow cytometry analysis of splenocytes isolated and stained with the fluorescently labeled anti-CD19-Alexa Fluor 700 antibodies from C57/BL56 mice sacrificed on day 12, where the splenocytes were isolated from the mice i.p. injected with C1498 cells (24 hours after the cells were transfected with STAV1 (3 μg/mL) for 3 hours and UV irradiated (120 mJ/cm for 1 minute) 44, or the UV irradiated cells (only) 45, and the PBS control 47 and boosted with the STAV1 after 6 days;

FIG. 13F shows flow cytometry analysis of splenocytes isolated and stained with the fluorescently labeled anti-CD49b-PE/Cy7 antibodies from C57/BL56 mice sacrificed on day 12, where the splenocytes were isolated from the mice i.p. injected with C1498 cells (24 hours after the cells were transfected with STAV1 (3 μg/mL) for 3 hours and UV irradiated (120 mJ/cm for 1 minute) 44, or the UV irradiated cells (only) 45, and the PBS control 47 and boosted with the STAV1 after 6 days;

FIG. 13G shows flow cytometry analysis of splenocytes isolated and stained with the fluorescently labeled anti-CD11b-FITC antibodies from C57/BL56 mice sacrificed on day 12, where the splenocytes were isolated from the mice i.p. injected with C1498 cells (24 hours after the cells were transfected with STAV1 (3 μg/mL) for 3 hours and UV irradiated (120 mJ/cm for 1 minute) 44, or the UV irradiated cells (only) 45, and the PBS control 47 and boosted with the STAV1 after 6 days;

FIG. 13H shows flow cytometry analysis of splenocytes isolated and stained with the fluorescently labeled anti-CD11c-FITC antibodies from C57/BL56 mice sacrificed on day 12, where the splenocytes were isolated from the mice i.p. injected with C1498 cells (24 hours after the cells were transfected with STAV1 (3 μg/mL) for 3 hours and UV irradiated (120 mJ/cm for 1 minute) 44, or the UV irradiated cells (only) 45, and the PBS control 47 and boosted with the STAV1 after 6 days;

FIG. 14A shows a schematic representation of the dead cell therapy in murine ALL model (EL4 cells) for a protocol of four (4) groups differing in the amount of STAVs transfected (PBS control (n=3) 12, 5×10⁶ cells/mouse (n=3) 72, 1×10⁶ cells/mouse (n=3) 74, 2×10⁵ cells/mouse) (n=3) 76, of mice I.V. sequentially injected every week with the irradiated T-ALL cells (EL4) using the MaxCyte GT system at 4 μg STAV/1×10⁶ cells and irradiated by UV (120 mJ/cm for 1 minute);

FIG. 14B shows flow cytometry analysis (performed using LSR-II) of the mouse T-ALL cells with anti-CD3-FITC, and anti-CD45-PacificBlue on day 38 (10 days after the last injection), where the splenocytes were isolated and stained with the different fluorescently labeled antibodies, where the PBS control (n=3) is shown as 12, 5×10⁶ cells/mouse (n=3) 72, 1×10⁶ cells/mouse (n=3) 74, 2×10⁵ cells/mouse) (n=3) 76;

FIG. 14C shows flow cytometry analysis (performed using LSR-II) of the mouse T-ALL cells with anti-CD3-FITC, and anti-CD4-PE on day 38 (10 days after the last injection), where the splenocytes were isolated and stained with the different fluorescently labeled antibodies, where the PBS control (n=3) is shown as 12, 5×10⁶ cells/mouse (n=3) 72, 1×10⁶ cells/mouse (n=3) 74, 2×10⁵ cells/mouse) (n=3) 76;

FIG. 14D shows flow cytometry analysis (performed using LSR-II) of the mouse T-ALL cells with anti-CD3-FITC, and anti-CD8a-PercP on day 38 (10 days after the last injection), where splenocytes were isolated/stained with the different fluorescently labeled antibodies, where PBS control (n=3) is 12, 5×10⁶ cells/mouse (n=3) 72, 1×10⁶ cells/mouse (n=3) 74, 2×10⁵ cells/mouse) (n=3) 76;

FIG. 14E shows flow cytometry analysis (performed using LSR-II) of the mouse T-ALL cells with anti-CD19-AlexaFluor 700 on day 38 (10 days after the last injection), where the splenocytes were isolated and stained with the different fluorescently labeled antibodies, where the PBS control (n=3) is shown as 12, 5×10⁶ cells/mouse (n=3) 72, 1×10⁶ cells/mouse (n=3) 74, 2×10⁵ cells/mouse) (n=3) 76;

FIG. 15A is a flow diagram showing a treatment protocol for cancer requiring treatment with a plurality of doses of leukemic cells treated with a plurality of STAVs selected from the group consisting of STAV1, STAV2, STAV3, STAV4 and STAV5, according to an embodiment of the invention;

FIG. 15B is a flow diagram showing a treatment protocol for cancer requiring treatment with a plurality of doses of leukemic cells treated with a plurality of STAVs selected from the group consisting of STAV1, STAV2, STAV3, STAV4 and STAV5 and a treatment with a Dendritic Cell vaccine generated with at least one STAV, according to an embodiment of the invention;

FIG. 15C is a flow diagram showing a treatment protocol for cancer requiring treatment with a plurality of doses of leukemic cells treated with a plurality of STAVs selected from the group consisting of STAV1, STAV2, STAV3, STAV4 and STAV5 and a treatment with a plurality of Dendritic Cell vaccines generated with a plurality of STAVs selected from the group consisting of STAV1, STAV2, STAV3, STAV4 and STAV5, according to an embodiment of the invention;

FIG. 15D is a flow diagram showing an alternative treatment protocol for cancer requiring treatment with a plurality of doses of leukemic cells treated with up to five STAVs comprising the group consisting of STAV1, STAV2, STAV3, STAV4 and STAV5 and a treatment with a plurality of Dendritic Cell vaccines generated with a plurality of STAVs selected from the group consisting of STAV1, STAV2, STAV3, STAV4 and STAV5, according to an embodiment of the invention; and

FIG. 16 is a flow diagram showing a limiting toxicity protocol for relapsed/refractory aggressive leukemia.

### DETAILED DESCRIPTION OF THE INVENTION

Listed below are definitions of various terms used in this application. These definitions apply to the terms as they are used throughout this specification and claims, unless otherwise limited in specific instances, either individually or as part of a larger group.

The transitional term ‘comprising’ is synonymous with ‘including’, ‘containing,’ or ‘characterized by’ is inclusive or open-ended and does not exclude additional, unrecited elements or method steps. The transitional phrase ‘consisting of’ excludes any element, step, or ingredient not specified in the claim, but does not exclude additional components or steps that are unrelated to the invention such as impurities ordinarily associated with a composition. The transitional phrase ‘consisting essentially of’ limits the scope of a claim to the specified materials or steps and those that do not materially affect the basic and novel characteristic(s) of the claimed invention.

The term ‘cancer’ includes, but is not limited to, the following cancers: epidermoid Oral: buccal cavity, lip, tongue, mouth, pharynx; Cardiac: sarcoma (angiosarcoma, fibrosarcoma, rhabdomyosarcoma, liposarcoma), myxoma, rhabdomyoma, fibroma, lipoma, and teratoma; Lung: bronchogenic carcinoma (squamous cell or epidermoid, undifferentiated small cell, undifferentiated large cell, adenocarcinoma), alveolar (bronchiolar) carcinoma, bronchial adenoma, sarcoma, lymphoma, chondromatous hamartoma, mesothelioma; Gastrointestinal: esophagus (squamous cell carcinoma, larynx, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel or small intestines (adenocarcinoma, lymphoma, carcinoid tumors, Karposi's sarcoma, leiomyoma, hemangioma, lipoma, neurofibroma, fibroma), large bowel or large intestines (adenocarcinoma, tubular adenoma, villous adenoma, hamartoma, leiomyoma), colon, colon-rectum, colorectal, rectum; Genitourinary tract: kidney (adenocarcinoma, Wilm's tumor (nephroblastoma), lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma); Liver: hepatoma (hepatocellular carcinoma), cholangiocarcinoma, hepatoblastoma, angiosarcoma, hepatocellular adenoma, hemangioma, biliary passages; Bone: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors; Nervous system: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma (pinealoma), glioblastoma multiform, oligodendroglioma, schwannoma, retinoblastoma, congenital tumors), spinal cord neurofibroma, meningioma, glioma, sarcoma); Gynecological: uterus (endometrial carcinoma), cervix (cervical carcinoma, pre-tumor cervical dysplasia), ovaries (ovarian carcinoma (serous cystadenocarcinoma, mucinous cystadenocarcinoma, unclassified carcinoma), granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma), breast; Hematologic: blood (myeloid leukemia (acute and chronic), acute lymphoblastic leukemia, chronic lymphocytic leukemia, myeloproliferative diseases, multiple myeloma, myelodysplastic syndrome), Hodgkin's disease, non-Hodgkin's lymphoma (malignant lymphoma) hairy cell; lymphoid disorders; Skin: malignant melanoma, basal cell carcinoma, squamous cell carcinoma, Karposi's sarcoma, keratoacanthoma, moles dysplastic nevi, lipoma, angioma, dermatofibroma, keloids, psoriasis, Thyroid gland: papillary thyroid carcinoma, follicular thyroid carcinoma; medullary thyroid carcinoma, undifferentiated thyroid cancer, multiple endocrine neoplasia type 2A, multiple endocrine neoplasia type 2B, familial medullary thyroid cancer, pheochromocytoma, paraganglioma; and adrenal glands: neuroblastoma. Thus, the term ‘cancerous cell’ as provided herein, includes a cell afflicted by any one of the above-identified conditions.

The term ‘subject’ as used herein refers to a mammal. A subject therefore refers to, for example, dogs, cats, horses, cows, sheep, pigs, guinea pigs, rats, mice, monkeys, apes and the like. Preferably the subject is a human. When the subject is a human, the subject may be referred to herein as a patient.

The words ‘treat’, ‘treating’ and ‘treatment’ refer to a method of alleviating or abating a disease and/or its attendant symptoms.

The words ‘preventing’ and ‘prevent’ describe reducing or eliminating the onset of the symptoms or complications of the disease, condition or disorder.

The terms ‘disease(s)’, ‘disorder(s)’, and ‘condition(s)’ are used interchangeably, unless the context clearly dictates otherwise.

The term ‘therapeutically effective amount’ of a compound or pharmaceutical composition of the application, as used herein, means a sufficient amount of the compound or pharmaceutical composition so as to decrease the symptoms of a disorder in a subject. As is well understood in the medical arts a therapeutically effective amount of a compound or pharmaceutical composition of this application will be at a reasonable benefit/risk ratio applicable to any medical treatment. It will be understood, however, that the total daily usage of the compounds and compositions of the present application will be decided by the attending physician within the scope of sound medical judgment. The specific modulatory (e.g., inhibitory or stimulatory) dose for any particular patient will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts.

As used herein, the phrase ‘pharmaceutically acceptable’ refers to those compounds, materials, compositions, carriers, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, the term ‘pharmaceutically acceptable salt’ refers to those salts of the compounds formed by the process of the present application which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge, et al. describes pharmaceutically acceptable salts in detail in J. Pharmaceutical Sciences, 66: 1-19 (1977), which is herein expressly incorporated by reference in its entirety and for all purposes. The salts can be prepared in situ during the final isolation and purification of the compounds of the application, or separately by reacting the free base or acid function with a suitable acid or base.

Examples of pharmaceutically acceptable salts include, but are not limited to, nontoxic acid addition salts: salts formed with inorganic acids such as hydrochloric acid, hydrobromic acid, phosphoric acid, sulfuric acid and perchloric acid, or with organic acids such as acetic acid, maleic acid, tartaric acid, citric acid, succinic acid or malonic acid. Other pharmaceutically acceptable salts include, but are not limited to, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, 7-toluenesulfonate, undecanoate, valerate salts, and the like. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counter ions such as halide, hydroxide, carboxylate, sulfate, phosphate, nitrate, alkyl having from 1 to 6 carbon atoms, sulfonate and aryl sulfonate.

As used herein, the term ‘pharmaceutically acceptable ester’ refers to esters of the compounds formed by the process of the present application which hydrolyze in vivo and include those that break down readily in the human body to leave the parent compound or a salt thereof. Suitable ester groups include, for example, those derived from pharmaceutically acceptable aliphatic carboxylic acids, particularly alkanoic, alkenoic, cycloalkanoic and alkanedioic acids, in which each alkyl or alkenyl moiety advantageously has not more than 6 carbon atoms. Examples of particular esters include, but are not limited to, formates, acetates, propionates, butyrates, acrylates and ethylsuccinates.

The term ‘pharmaceutically acceptable prodrugs’ as used herein, refers to those prodrugs of the compounds formed by the process of the present application which are, within the scope of sound medical judgment, suitable for use in contact with the tissues of humans and lower animals with undue toxicity, irritation, allergic response, and the like, commensurate with a reasonable benefit/risk ratio, and effective for their intended use, as well as the zwitterionic forms, where possible, of the compounds of the present application.

‘Prodrug’, as used herein, means a compound which is convertible in vivo by metabolic means (e.g., by hydrolysis) to afford any compound delineated by the formulae of the instant application. Various forms of prodrugs are known in the art, for example, as discussed in Bundgaard, (ed.), Design of Prodrugs, Elsevier (1985); Widder, et al. (ed.), Methods in Enzymology, vol. 4, Academic Press (1985); Krogsgaard-Larsen, et al., (ed). “Design and Application of Prodrugs, Textbook of Drug Design and Development, Chapter 5, 113-191 (1991); Bundgaard, et al., Journal of Drug Deliver Reviews, 8:1-38(1992); Bundgaard, J. of Pharmaceutical Sciences, 77:285 et seq. (1988); Higuchi and Stella (eds.) Prodrugs as Novel Drug Delivery Systems, American Chemical Society (1975); and Bernard Testa & Joachim Mayer, “Hydrolysis In Drug And Prodrug Metabolism: Chemistry, Biochemistry And Enzymology,” John Wiley and Sons, Ltd. (2002), which are all herein expressly incorporated by reference in their entireties and for all purposes.

‘Pharmaceutically acceptable excipient’ means an excipient that is useful in preparing a pharmaceutical composition that is generally safe, non-toxic and neither biologically nor otherwise undesirable, and includes an excipient that is acceptable for veterinary use as well as human pharmaceutical use. A pharmaceutically acceptable excipient as used in the specification and claims includes both one and more than one such excipient.

This application also encompasses pharmaceutical compositions containing, and methods of treating disorders through administering, pharmaceutically acceptable prodrugs of compounds of the application. For example, compounds of the application having free amino, amido, hydroxy or carboxylic groups can be converted into prodrugs. Prodrugs include compounds wherein an amino acid residue, or a polypeptide chain of two or more (e.g., two, three or four) amino acid residues is covalently joined through an amide or ester bond to a free amino, hydroxy or carboxylic acid group of compounds of the application. The amino acid residues include but are not limited to the 20 naturally occurring amino acids commonly designated by three letter symbols and also includes 4-hydroxyproline, hydroxylysine, demosine, isodemosine, 3-methylhistidine, norvalin, beta-alanine, gamma-aminobutyric acid, citrulline, homocysteine, homoserine, ornithine and methionine sulfone. Additional types of prodrugs are also encompassed. For instance, free carboxyl groups can be derivatized as amides or alkyl esters. Free hydroxy groups may be derivatized using groups including but not limited to hemisuccinates, phosphate esters, dimethylaminoacetates, and phosphoryloxymethyloxy carbonyls, as outlined in Advanced Drug Delivery Reviews, 1996, 19, 1-15, which is herein expressly incorporated by reference in its entirety and for all purposes. Carbamate prodrugs of hydroxy and amino groups are also included, as are carbonate prodrugs, sulfonate esters and sulfate esters of hydroxy groups. Derivatization of hydroxy groups as (acyloxy)methyl and (acyloxy)ethyl ethers wherein the acyl group may be an alkyl ester, optionally substituted with groups including but not limited to ether, amine and carboxylic acid functionalities, or where the acyl group is an amino acid ester as described above, are also encompassed. Prodrugs of this type are described in J. Med. Chem. 1996, 39, 10, which is herein expressly incorporated by reference in its entirety and for all purposes. Free amines can also be derivatized as amides, sulfonamides or phosphonamides. All of these prodrug moieties may incorporate groups including but not limited to ether, amine and carboxylic acid functionalities.

Combinations of substituents and variables envisioned by this application are only those that result in the formation of stable compounds. The term ‘stable’, as used herein, refers to compounds which possess stability sufficient to allow manufacture and which maintains the integrity of the compound for a sufficient period of time to be useful for the purposes detailed herein (e.g., therapeutic or prophylactic administration to a subject).

When any variable (e.g., R₁) occurs more than one time in any constituent or formula for a compound, its definition at each occurrence is independent of its definition at every other occurrence. Thus, for example, if a group is shown to be substituted with one or more R moieties, then R at each occurrence is selected independently from the definition of R. Also, combinations of substituents and/or variables are permissible, but only if such combinations result in stable compounds within a designated atom's normal valency.

In addition, some of the compounds of this application have one or more double bonds, or one or more asymmetric centers. Such compounds can occur as racemates, racemic mixtures, single enantiomers, individual diastereomers, diastereomeric mixtures, and cis- or trans- or E- or Z-double isomeric forms, and other stereoisomeric forms that may be defined, in terms of absolute stereochemistry, as (R)- or (S)-, or as (D)- or (L)- for amino acids. When the compounds described herein contain olefinic double bonds or other centers of geometric asymmetry, and unless specified otherwise, it is intended that the compounds include both E and Z geometric isomers. The configuration of any carbon-carbon double bond appearing herein is selected for convenience only and is not intended to designate a particular configuration unless the text so states; thus a carbon-carbon double bond depicted arbitrarily herein as trans may be cis, trans, or a mixture of the two in any proportion. All such isomeric forms of such compounds are expressly included in the present application.

‘Isomerism’ means compounds that have identical molecular formulae but differ in the sequence of bonding of their atoms or in the arrangement of their atoms in space. Isomers that differ in the arrangement of their atoms in space are termed ‘stereoisomers’. Stereoisomers that are not mirror images of one another are termed ‘diastereoisomers’, and stereoisomers that are non-superimposable mirror images of each other are termed ‘enantiomers’ or sometimes optical isomers. A mixture containing equal amounts of individual enantiomeric forms of opposite chirality is termed a ‘racemic mixture’.

A carbon atom bonded to four non-identical substituents is termed a ‘chiral center’.

‘Chiral isomer’ means a compound with at least one chiral center. Compounds with more than one chiral center may exist either as an individual diastereomer or as a mixture of diastereomers, termed ‘diastereomeric mixture’. When one chiral center is present, a stereoisomer may be characterized by the absolute configuration (R or S) of that chiral center, e.g., carbon. Absolute configuration refers to the arrangement in space of the substituents attached to the chiral center. The substituents attached to the chiral center under consideration are ranked in accordance with the Sequence Rule of Cahn, Ingold and Prelog. (Cahn et al., Angew. Chem. Inter. Edit. 1966, 5, 385; errata 511; Cahn et al., Angew. Chem. 1966, 78, 413; Cahn and Ingold, J. Chem. Soc. 1951 (London), 612; Cahn et al., Experientia 1956, 12, 81; Cahn, J. Chem. Educ. 1964, 41, 116).

‘Geometric isomer’ means the diastereomers that owe their existence to hindered rotation about double bonds. These configurations are differentiated in their names by the prefixes cis and trans, or Z and E, which indicate that the groups are on the same or opposite side of the double bond in the molecule according to the Cahn-Ingold-Prelog rules.

Furthermore, the structures and other compounds discussed in this application include all atropic isomers thereof. ‘Atropic isomers’ are a type of stereoisomer in which the atoms of two isomers are arranged differently in space. Atropic isomers owe their existence to a restricted rotation caused by hindrance of rotation of large groups about a central bond. Such atropic isomers typically exist as a mixture, however as a result of recent advances in chromatography techniques; it has been possible to separate mixtures of two atropic isomers in select cases.

‘Tautomer’ is one of two or more structural isomers that exist in equilibrium and is readily converted from one isomeric form to another. This conversion results in the formal migration of a hydrogen atom accompanied by a switch of adjacent conjugated double bonds. Tautomers exist as a mixture of a tautomeric set in solution. In solid form, usually one tautomer predominates. In solutions where tautomerization is possible, a chemical equilibrium of the tautomers will be reached. The exact ratio of the tautomers depends on several factors, including temperature, solvent and pH. The concept of tautomers that are interconvertable by tautomerizations is called tautomerism.

Of the various types of tautomerism that are possible, two are commonly observed. In keto-enol tautomerism a simultaneous shift of electrons and a hydrogen atom occurs. Ring-chain tautomerism arises as a result of the aldehyde group (—CHO) in a sugar chain molecule reacting with one of the hydroxy groups (—OH) in the same molecule to give it a cyclic (ring-shaped) form as exhibited by glucose. Common tautomeric pairs are: ketone-enol, amide-nitrile, lactam-lactim, amide-imidic acid tautomerism in heterocyclic rings (e.g., in nucleobases such as adenine, guanine, thymine and cytosine), amine-enamine and enamine-enamine. The compounds of this application may also be represented in multiple tautomeric forms, in such instances, the application expressly includes all tautomeric forms of the compounds described herein (e.g., alkylation of a ring system may result in alkylation at multiple sites, the application expressly includes all such reaction products).

In the present application, the structural formula of the compound represents a certain isomer for convenience in some cases, but the present application includes all isomers, such as geometrical isomers, optical isomers based on an asymmetrical carbon, stereoisomers, tautomers, and the like. In the present specification, the structural formula of the compound represents a certain isomer for convenience in some cases, but the present application includes all isomers, such as geometrical isomers, optical isomers based on an asymmetrical carbon, stereoisomers, tautomers, and the like.

Additionally, the compounds of the present application, for example, the salts of the compounds, can exist in either hydrated or unhydrated (the anhydrous) form or as solvates with other solvent molecules. Non-limiting examples of hydrates include monohydrates, dihydrates, etc. Non-limiting examples of solvates include ethanol solvates, acetone solvates, etc.

‘Solvate’ means solvent addition forms that contain either stoichiometric or non stoichiometric amounts of solvent. Some compounds have a tendency to trap a fixed molar ratio of solvent molecules in the crystalline solid state, thus forming a solvate. If the solvent is water the solvate formed is a hydrate; and if the solvent is alcohol, the solvate formed is an alcoholate. Hydrates are formed by the combination of one or more molecules of water with one molecule of the substance in which the water retains its molecular state as H₂O

In the present specification, the structural formula of the compound represents a certain isomer for convenience in some cases, but the present application includes all isomers, such as geometrical isomers, optical isomers based on an asymmetrical carbon, stereoisomers, tautomers, and the like.

All documents mentioned herein are incorporated herein by reference. All publications and patent documents cited in this application are incorporated by reference for all purposes to the same extent as if each individual publication or patent document were so individually denoted. By their citation of various references in this document, Applicants do not admit any particular reference is ‘prior art’ to their invention. Embodiments of inventive compositions and methods are illustrated in the following examples.

STING is a cellular innate immune receptor essential for controlling the transcription of numerous host defense genes, including type I IFN and pro-inflammatory cytokines following the recognition of CDN's or aberrant DNA species in the cytosol of the cell. The source of DNA can comprise the genome of invading pathogens such as herpes simplex 1 virus (HSV1) or CDNs which are known to be secreted by bacteria such as Listeria monocytogenes. That is, STING can directly sense CDNs including c-di-GMP or c-di-AMP secreted by invading intracellular bacteria, cyclic-GMP-AMP (cGAMP) generated by the cellular synthase, Cyclic GMP-AMP synthase (cGAS) following association with cytosolic dsDNA species such as microbial DNA, or self-DNA. Generally, the cytosol of the cell is free of DNA, since it can aggravate STING-dependent cytokine production, an event that can lead to lethal auto-inflammatory disease. For example, self-DNA leaked from the nucleus of cells, following cell division or following DNA damage, is prevented from activating STING signaling by the exonuclease DNase III (Trex1). Consequently, defects in Trex1 function can lead to severe auto-inflammatory diseases due to undigested self-DNA triggering STING activity. In addition, following the engulfment of apoptotic cells, phagocyte-dependent DNase II plays a critical role in digesting the DNA from the dead cell, to prevent it from activating STING-signaling extrinsically within the phagocyte.

As used herein the phrase ‘STING intracellular pathways’ include IRF-3 and NF-kB pathways. In the absence of any contrary indication in the specification, the use of the term approximately means plus or minus ten percent, e.g., approximately 200 minutes means 200 plus or minus 20 minutes.

Self-DNA leaked from the nucleus of the host cell, following cell division or even as a consequence of DNA damage can activate STING. Such self-DNA may be responsible for causing a variety of auto-inflammatory disease such as Systemic Lupus Erythamatosis (SLE) or Aicardi-Goutieres Syndrome (AGS) and may even be associated with inflammation-associated cancer. Recent insight into the regulation of STING signaling has generated much needed information relating to the causes of inflammatory disease, providing new opportunities to develop novel anti-inflammatory compounds that target this pathway.

The ability of dying cells to activate Antigen Presenting Cells (APCs) is carefully controlled to avoid unwanted inflammatory responses. For example, following phagocytosis, regular dying cells do not trigger inflammatory responses which can be required for efficient cytotoxic T lymphocyte (CTL) priming of the immune system.

Tumor cells presumably mimic these processes to avoid activating APCs. However, dying tumor cells contain exogenous innate immune agonists such as cytosolic DNA. The cytosolic DNA can potently activate APCs in trans through extrinsic innate immune, STING-dependent signaling, to generate potent Cytotoxic T Lymphocyte (CTL) activity. In the absence of STING agonists, dying cells are ineffectual in the stimulation of APCs in trans. Indeed, cytosolic STING activators, including cytosolic DNA and cyclic dinucleotides (CDNs), constitute cellular danger associated molecular patterns (DAMPs) usually only generated by viral infection or following DNA-damage events, that can render tumor cells highly immunogenic (i.e., STING activators make a ‘cold’ tumor ‘hot’).

Thus, the efficient eradication of apoptotic cells is designed to avoid invoking an inflammatory event. Dying cells are generally poor activators of phagocytes and are immunologically indolent due to the genomic DNA being degraded by host DNases to prevent the intrinsic and extrinsic activation of STING. Tumor cells mimic this efficient process and avoid activating anti-tumor CTL activity. However, cancer cells containing cytosolic dsDNA species, that escape degradation, can potently stimulate APCs, via extrinsic STING-signaling, to promote the cross-presentation of tumor antigen.

STING is activated by cyclic dinucleotides (CDNs) such as cyclic di-GMP and cyclic-di-AMP secreted by intracellular bacteria following infection. Alternatively, STING can be activated by cyclic GMP-AMP (cGAMP) generated by a cellular cGAMP synthase cGAS after association with aberrant cytosolic dsDNA species, which can include microbial DNA or self-DNA leaked from the nucleus. Notably, STING signaling has been shown to be important for facilitating anti-tumor T cell activity. Cytosolic dsDNA species present within a dying tumor cell can activate extrinsic STING signaling in phagocytes likely following association with cGAS which can generate CDNs.

The phrase ‘STing Dependent AdjuVants’ or ‘STAVs’ refers to dsDNA oligonucleotides of 70 bp which are innate immune activators of STING. The STAV compositions of the present invention comprise at least one modification which confers increased or enhanced stability to the STAVs, including, for example, improved resistance to nuclease digestion in vivo. In an embodiment, the STAV compositions of the present invention have undergone a chemical or biological modification to render them more stable. Exemplary modifications to the STAVs include the modification of a base, for example, the chemical modification of a base.

The term ‘functional’ as used herein means that the STAV has biological activity to activate STING. The STA compositions of the invention are useful for the treatment of cancer, inflammation and other disorders. The term ‘therapeutic levels’ refers to levels of STAVs above normal physiological levels, or the levels in the subject prior to administration of the STAV composition. As provided herein, the compositions include a transfer vehicle. As used herein, the term ‘transfer vehicle’ includes any of the standard pharmaceutical carriers, diluents, excipients and the like which are generally intended for use in connection with the administration of biologically active agents, including nucleic acids. The compositions and in particular the transfer vehicles described herein are capable of delivering STAVs to the target cell. In embodiments, the transfer vehicle is a lipid nanoparticle.

The term ‘complimentary’ when referring to dsDNA as used herein means traditional Watson and Crick complementary, at least approximately eighty (80) percent, where approximately in this range means plus or minus twenty (20) percent. The phrase ‘the first strand comprises at least eighty percent complimentary nucleobases with respect to the second strand’ implies that a first strand that contains 76 nucleobases of which 61 nucleobases are adenine means that the second strand contains at least 61 nucleobases that are thymine.

The term ‘polymer-conjugated lipid’ means a polymer (for example, polyethylene glycol (PEG), polypropylene glycol, polyvinvylpyrrolidone, poly(N-(2-hydroxypropyl)methacrylamide)s and PEGylated liposomes with different functional groups, including methoxy (OCH₃), amino (NH₂), carboxyl (COOH), and hydroxyl (OH) moieties) conjugated with a lipid. For example PEG can be conjugated with myristoyl diglyceride to generate DMG-PEG 2000. Alternatively, PEG can be conjugated with DSPE a water soluble derivative of phosphatidylethanolamine with (18:0) stearic acid acyl chains to generate DSPE PEG 2000. PEG conjugated lipids can incorporate various functionalized PEG terminal groups including amine, carboxylic acid, azide, aldehyde, thiol, and hydroxyl moieties. PEG conjugated lipids improve circulation times, drug stability, suitability of different routes of administration, and help achieve targeted drug delivery. In an alternative embodiment of the present invention, a branched polymer (e.g., poly(oligo(ethylene glycol) methyl ether methacrylate, i.e., poly(tri(ethylene glycol) methyl ether methacrylate, poly(tetra(ethylene glycol) methyl ether methacrylate, poly(penta(ethylene glycol) methyl ether methacrylate, poly(hexa(ethylene glycol) methyl ether methacrylate, poly(hepta(ethylene glycol) methyl ether methacrylate, poly(octa(ethylene glycol) methyl ether methacrylate, poly(noan(ethylene glycol) methyl ether methacrylate) can be conjugated with lipids.

In addition to PEG conjugated lipids, a ‘sterol’ or an unsaturated steroid alcohol, can be used to enhance the stability of the LNP. Sterols can include natural sterols and sterols with unnatural ring junctions. Sterols can be used to assist the efficiency of introducing the STAV into the cells. Changing the nature of the sterol component can also be used to alter the efficiency of introducing the STAV into cells. Natural sterols include cholesterol, cholesterol sulfate, desmosterol, stigmasterol, lanosterol, 7-dehydrocholesterol, dihydrolanosterol, zymosterol, lathosterol, 14-demethyl-lanosterol, 8(9)-dehydrocholesterol, 8(14)-dehydrocholesterol, FF-MAS, diosgenin, dehydroepiandrosterone (DHEA) sulfate, DHEA, sitosterol, lanosterol-95, zymostenol, sitostanol, campestanol, campesterol, 7-dehydrodesmosterol, pregnenolone, dihydro T-MAS, delta 5-avenasterol, brassicasterol, dihydro FF-MAS, 24-methylene cholesterol, 3B-hydroxy-7-oxo-5-cholestenoic acid, 7α-hydroxy-3-oxo-4-cholestenoic acid, 3B,7α-dihydroxy-5-cholestenoic acid, 3B,7B-dihydroxy-5-cholestenoic acid, 3B-hydroxy-5-cholestenoic acid, 3-oxo-4-cholestenoic acid, 3B,7α,24S-trihydroxy-5-cholestenoic acid, 3B,24S-dihydroxy-5-cholestenoic acid, 3B,7α,25-trihydroxy-5-cholestenoic acid, and 30,25-OH-7-oxo-5-cholestenoic acid.

A ‘phospholipid’ means a molecule with a hydrophilic head group and an aliphatic chain linked to an alcohol moiety. The nature of the head group, the aliphatic chain and the alcohol can be used to generate a wide variety of phospholipids. The aliphatic chain includes saturated acyl chains, saturated alkyl chains, unsaturated acyl chains, unsaturated alkyl chains, saturated acyl chains with ether bonds, saturated alkyl chains with ester bonds, unsaturated acyl chains with ether bonds and unsaturated alkyl chains with ester bonds. Glycerophospholipids and sphingomyelins are phospholipids which differ based on the alcohol moieties. Phospholipids include phosphatidylcholine, phosphatidylethanolamine, phosphatidylserine, phosphatidic acid, phosphatidylinositol, phosphatidylglycerol, cardiolipin, dipalmitoyl, dimyristoyl, DSPC, dioleoyl, and L-α-phosphatidylcholine. In an embodiment of the present invention, the alcohol in the phospholipid can be a C₃ alcohol. In an alternative embodiment of the present invention, the phospholipid can include a C₄-C₈ alcohol.

An ‘ionizing lipid’ is a class of lipid molecules which remain neutral at physiological pH, but are protonated under acidic conditions. Ionizing lipids promote endosome escape and reduce toxicity of the LNP. Ionizable lipids include 7-[(2-Hydroxyethyl)[8-(nonyloxy)-8-oxooctyl]amino]heptyl 2-octyldecanoate, DODMA (MBN 305A), DLin-KC2-DMA, (6Z,9Z,28Z,31Z)-Heptatriaconta-6,9,28,31-tetraen-19-yl 4-(dimethylamino)butanoate (D-Lin-MC3-DMA, or MC3), Heptadecan-9-yl 8-{(2-hydroxyethyl)[6-oxo-6-(undecyloxy)hexyl]amino}octanoate (SM-102), and [(4-Hydroxybutyl)azanediyl]di(hexane-6,1-diyl) bis(2-hexyldecanoate) (ALC-0315).

A ‘cationic lipid’ is a class of lipid molecules that are positively charged amphiphiles consisting of three basic chemical functional domains: a hydrophilic head, a hydrophobic tail, and a tether between the hydrophilic head and the hydrophobic tail. Cationic lipids include DOTAP, dimethyldioctadecylammonium bromide, 33-[N-(N′,N′-dimethylaminoethane)-carbamoyl]cholesterol, dimethyldioctadecylammonium, 1,2-dimyristoyl-3-trimethylammonium-propane, 1,2-stearoyl-3-trimethylammonium-propane and N-(4-carboxybenzyl)-N,N-dimethyl-2,3-bis(oleoyloxy)propan-1-aminium.

The term ‘LNP’ means a lipid nanoparticle. A LNP represents a particle made from lipids (e.g., cationic lipids, non-cationic lipids, conjugated lipids and/or a sterol that prevents aggregation of the nanoparticle), and a STAV, where the STAV is encapsulated within the lipid (e.g., Nano-STAVs) (the LNPs used in the Nano-STAVs described herein were synthesized by Precision Nanosystems, South San Francisco, Calif. 94080).

In an embodiment of the present invention, LNP formulations can have four major components, other than the nucleic acid. In an embodiment of the present invention, a LNP comprises a phospholipid, a sterol, an ionizable lipid, and a polymer-conjugated lipid. In an alternative embodiment of the present invention, a LNP comprises a phospholipid, a sterol, a cationic lipid, and a polymer-conjugated lipid. In an embodiment of the present invention, the cationic lipid can be DOTAP. In an embodiment of the present invention, the phospholipid can be DSPC. In an embodiment of the present invention, the sterol can be cholesterol. In an embodiment of the present invention, the ionizable lipid can be MC3. In an embodiment of the present invention, the polymer conjugated lipid can be DMG-PEG 2000. In an embodiment of the present invention, DSPC, cholesterol, MC3 and DMG-PEG 2000 can be used to generate the LNP to be combined with STAVB1, STAV2 or STAV3 to generate Nano-STAV1, Nano-STAV2 and Nano-STAV3 respectively, where the diameter of the spherical LNPs can be approximately 88 nm, where approximately means+−10 nm. In an embodiment of the present invention, the cholesterol can be between 35-45% of the LNP composition. In an embodiment of the present invention, the LNP comprises a DSPC, cholesterol, an MC3-like lipid and a PEG-conjugated lipid. The phospholipid and cholesterol promote stability and structural integrity of the LNP. The ionizable lipid promotes electrostatic interaction with the negatively charged nucleic acids and assists intracellular delivery. The polymer-conjugated lipid improves solubility of the LNP in serum, and circulation by preventing the particles from aggregating, while retaining good biocompatibility and having good tolerance characteristics. In an embodiment of the present invention, the Nano-STAVs were composed of 76 bp of dsDNA modified with ps to block exonuclease activity, encapsulated at a nitrogen to phosphate mole ratio of approximately 6 (where approximately means plus or minus one). In an embodiment of the present invention, the Nano-STAVs can be approximately 100 nm in size, where approximately means plus or minus ten (10) percent. In an embodiment of the present invention, the STAVS are approximately 50% encapsulated in the Nano-STAVS. In this range approximately means plus or minus twenty (20) percent. In an alternative embodiment of the present invention, the STAVS are approximately 75% encapsulated in the Nano-STAVS. In this range approximately means plus or minus ten (10) percent. In another embodiment of the present invention, the STAVS are at least approximately 90% encapsulated in the Nano-STAVS. In this range approximately means plus or minus five (5) percent. In another alternative embodiment of the present invention, the STAVS are approximately 98% encapsulated in the Nano-STAVS. In this range approximately means plus or minus one (1) percent. In an embodiment of the present invention, the STAVS are approximately 98% encapsulated in the Nano-STAVS, at a concentration of dsDNA in the LNP in PBS of 0.2 mg/mL. LNP can be extremely useful for systemic applications, as they can exhibit extended circulation lifetimes following i.v. injection, they can accumulate at distal sites, and they can deliver the STAVs at sites distal to the site of administration.

The methods of the invention provide for optional co-delivery of one or more unique STAVs to target cells, for example, by combining two unique STAVs into a single transfer vehicle. In an embodiment of the present invention, a therapeutic first STAV, and a therapeutic second STAV, can be formulated in a single transfer vehicle and administered. The present invention also contemplates co-delivery and/or co-administration of a therapeutic first STAV and a second STAV to facilitate and/or enhance the function or delivery of one or both the therapeutic first STAV and the therapeutic second STAV.

Pharmaceutical compositions including a compound of the present application in free form or in a pharmaceutically acceptable salt form in association with at least one pharmaceutically acceptable carrier or diluent may be manufactured in a conventional manner by mixing, granulating or coating methods. For example, oral compositions can be tablets or gelatin capsules comprising the active ingredient together with a) diluents, e.g., lactose, dextrose, sucrose, mannitol, sorbitol, cellulose and/or glycine; b) lubricants, e.g., silica, talcum, stearic acid, its magnesium or calcium salt and/or polyethyleneglycol; for tablets also c) binders, e.g., magnesium aluminum silicate, starch paste, gelatin, tragacanth, methylcellulose, sodium carboxymethylcellulose and or polyvinylpyrrolidone; if desired d) disintegrants, e.g., starches, agar, alginic acid or its sodium salt, or effervescent mixtures; and/or e) absorbents, colorants, flavors and sweeteners. Injectable compositions can be aqueous isotonic solutions or suspensions, and suppositories can be prepared from fatty emulsions or suspensions. The compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances. Suitable formulations for transdermal applications include an effective amount of a compound of the present application with a carrier. A carrier may include absorbable pharmacologically acceptable solvents to assist passage through the skin of the host. For example, transdermal devices may be in the form of a bandage comprising a backing member, a reservoir containing the compound optionally with carriers, optionally a rate controlling barrier to deliver the compound to the skin of the host at a controlled and predetermined rate over a prolonged period of time, and means to secure the device to the skin. Matrix transdermal formulations may also be used. Suitable formulations for topical application, e.g., to the skin and eyes, are preferably aqueous solutions, ointments, creams or gels well-known in the art. Such may contain solubilizers, stabilizers, tonicity enhancing agents, buffers and preservatives.

The pharmaceutical compositions of the present application comprise a therapeutically effective amount of a compound of the present application formulated together with one or more pharmaceutically acceptable carriers. As used herein, the term ‘pharmaceutically acceptable carrier’ means a non-toxic, inert solid, semi-solid or liquid filler, diluent, encapsulating material or formulation auxiliary of any type. Some examples of materials which may serve as pharmaceutically acceptable carriers include, but are not limited to, ion exchangers, alumina, aluminum stearate, lecithin, serum proteins, such as human serum albumin, buffer substances such as phosphates, glycine, sorbic acid, or potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, polyacrylates, waxes, polyethylenepolyoxy propylene-block polymers, wool fat, sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes, oils such as peanut oil, cottonseed oil; safflower oil; sesame oil; olive oil; corn oil and soybean oil; glycols such a propylene glycol or polyethylene glycol; esters such as ethyl oleate and ethyl laurate, agar; buffering agents such as magnesium hydroxide and aluminum hydroxide; alginic acid; pyrogen-free water, isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

The pharmaceutical compositions of this application may be administered to humans and other animals orally, rectally, parenterally, intracisternally, intravaginally, intraperitoneally, topically (as by powders, ointments, or drops), buccally, or as an oral or nasal spray.

Liquid dosage forms for oral administration may include pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous, or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

In order to prolong the effect of a drug, it is often desirable to slow the absorption of the drug from s.c. or intramuscular injection. This may be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle.

Compositions for rectal or vaginal administration are preferably suppositories which can be prepared by mixing the compounds of this application with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid compositions of a similar type may also be employed as fillers in soft and hard filled gelatin capsules using such excipients as lactose or milk sugar as well as high molecular weight polyethylene glycols and the like.

The active compounds may also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents.

Dosage forms for topical or transdermal administration of a compound of this application include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, eye ointments, powders and solutions are also contemplated as being within the scope of this application.

The ointments, pastes, creams and gels may contain, in addition to an active compound of this application, excipients such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to the compounds of this application, excipients such as lactose, talc, silicic acid, aluminum hydroxide, calcium silicates and polyamide powder, or mixtures of these substances. Sprays can additionally contain customary propellants such as chlorofluorohydrocarbons.

Transdermal patches have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

For any compound, the therapeutically effective amount can be estimated initially either in cell culture assays, e.g., of neoplastic cells, or in animal models, usually rats, mice, rabbits, dogs, or pigs. The animal model may also be used to determine the appropriate concentration range and route of administration. Such information can then be used to determine useful doses and routes for administration in humans. Therapeutic/prophylactic efficacy and toxicity may be determined by standard pharmaceutical procedures in cell cultures or experimental animals, e.g., ED₅₀ (the dose therapeutically effective in 50% of the population) and LD₅₀ (the dose lethal to 50% of the population). The dose ratio between toxic and therapeutic effects is the therapeutic index, and it can be expressed as the ratio, LD₅₀/ED₅₀. Pharmaceutical compositions that exhibit large therapeutic indices are preferred. The dosage may vary within this range depending upon the dosage form employed, sensitivity of the patient, and the route of administration.

Dosage and administration are adjusted to provide sufficient levels of the active agent(s) or to maintain the desired effect. Factors which may be taken into account include the severity of the disease state, general health of the subject, age, weight, and gender of the subject, diet, time and frequency of administration, drug combination(s), reaction sensitivities, and tolerance/response to therapy. Long-acting pharmaceutical compositions may be administered every 3 to 4 days, every week, or once every two weeks depending on half-life and clearance rate of the particular formulation.

The quantity of active ingredient (e.g., a formulation of the disclosed compound or salt, hydrate, solvate or isomer thereof) in a unit dose of composition is an effective amount and is varied according to the particular treatment involved. One skilled in the art will appreciate that it is sometimes necessary to make routine variations to the dosage depending on the age and condition of the patient. The dosage will also depend on the route of administration. A variety of routes are contemplated, including oral, pulmonary, rectal, parenteral, transdermal, s.c., i.v., intramuscular, intraperitoneal, inhalational, buccal, sublingual, intrapleural, intrathecal, intranasal, and the like. Dosage forms for the topical or transdermal administration of a compound of this application include powders, sprays, ointments, pastes, creams, lotions, gels, solutions, patches and inhalants. In one embodiment, the active compound is mixed under sterile conditions with a pharmaceutically acceptable carrier, and with any preservatives, buffers or propellants that are required.

The pharmaceutical compositions containing active compounds of the present application may be manufactured in a manner that is generally known, e.g., by means of conventional mixing, dissolving, granulating, dragee-making, levigating, emulsifying, encapsulating, entrapping, or lyophilizing processes. Pharmaceutical compositions may be formulated in a conventional manner using one or more pharmaceutically acceptable carriers comprising excipients and/or auxiliaries that facilitate processing of the active compounds into preparations that can be used pharmaceutically. Of course, the appropriate formulation is dependent upon the route of administration chosen.

Techniques for formulation and administration of the disclosed compounds of the application can be found in Remington: the Science and Practice of Pharmacy, 19t edition, Mack Publishing Co., Easton, Pa. (1995), which is herein expressly incorporated by reference in its entirety and for all purposes. In an embodiment, the compounds described herein, and the pharmaceutically acceptable salts thereof, are used in pharmaceutical preparations in combination with a pharmaceutically acceptable carrier or diluent. Suitable pharmaceutically acceptable carriers include inert solid fillers or diluents and sterile aqueous or organic solutions. The compounds will be present in such pharmaceutical compositions in amounts sufficient to provide the desired dosage amount in the range described herein.

Therapies based on the use CDNs as anti-tumor agents have been tested. In this scenario, CDNs are directly inoculated into tumors which plausibly stimulate APC activity to augment anti-tumor T cells responses. However, while working robustly in murine tumor models, the CDNs have exhibited little effect in human cancer trials, almost certainly due to their high turnover rate, in vivo. This has led to the generation of non-nucleotide-based STING agonists (small drugs), which may be able to escape degradation more effectively. STING signaling in the context of combined treatment with checkpoint inhibitors found that the therapeutic effect of an immune checkpoint inhibitory receptor (CTLA-4) and anti-PD-L1 monoclonal antibodies was lost in STING-deficient mice. In an embodiment of the present invention, STAVs represent a new generation of innate immune activators that trigger STING signaling.

Retrieved tumor cells transfected with STAVs activate APCs in trans and can generate potent anti-tumor T cell activity. Immunocompetent mice bearing metastatic syngeneic tumors can be treated with STAV ‘loaded’ tumor cells after reinfusion and inoculation. Select leukemias, such as acute myeloid leukemia (AML), acute lymphocytic leukemia (ALL) and adult T cell leukemia (ALL) can theoretically be amenable to treatment with STAVs. Further, the range of cancers can be extended to include melanomas and cutaneous T cell lymphomas. The i.t. inoculation of syngeneic melanoma tumors (B16) in immunocompetent mice can be used to generate effective anti-tumor CTL activity and cause tumor regression. However, in situations where it is not feasible to retrieve sufficient tumor cells to carry out the transfection with STAVs for re-infusion, the STAV based approach may not be applicable.

In an embodiment of the present invention, the direct introduction of the STAVs into the tumor microenvironment (TME) can represent a significant advance. Further, in various embodiments of the present invention, the range of cancers amenable to STAV therapy can be extended using a non-cell based LNP strategy that effectively delivers high concentrations of Nano-STAVs into the TME to potently generate anti-tumor cytotoxic T cell activity. In an embodiment of the present invention, the tumor regression generated by Nano-STAVs can be augmented by co-delivery of checkpoint inhibitors.

In an embodiment of the present invention, data indicates that Nano-STAVs are a potent anti-tumor therapy that suppresses the growth of localized tumors (B16 melanoma model in C57/BL6 mice). In an embodiment of the present invention, the tumor regression effect was greatly augmented with the synergistic addition of checkpoint inhibitors. In an embodiment of the present invention, the activation of STING signaling in APC's is a main mechanism of generating anti-tumor T cell activity and is capable of overcoming resistance to checkpoint therapy. In an embodiment of the present invention, the benefit of Nano-STAVs over small drug agonists is that the procedure mimics the normal process of antigen cross-presentation, is non-toxic, simple, and inexpensive.

Some of the foregoing compounds can comprise one or more asymmetric centers, and thus can exist in various isomeric forms, e.g., stereoisomers and/or diastereomers. Accordingly, compounds of the application may be in the form of an individual enantiomer, diastereomer or geometric isomer, or may be in the form of a mixture of stereoisomers. In one embodiment, the compounds of the application are enantiopure compounds. In another embodiment, mixtures of stereoisomers or diastereomers are provided.

Another aspect is an isotopically labeled compound of any of the formulae delineated herein. Such compounds have one or more isotope atoms which may or may not be radioactive (e.g., ³H, ²H, ⁴C, ¹³C, ¹⁸F, ³⁵S, ³²P, ¹²⁵I, and ¹³¹I) introduced into the compound. Such compounds are useful for drug metabolism studies and diagnostics, as well as therapeutic applications.

Potency can also be determined by IC₅₀ value. A compound with a lower IC₅₀ value, as determined under substantially similar conditions, is more potent relative to a compound with a higher IC₅₀ value. In some embodiments, the substantially similar conditions comprise determining the level of binding of a known STING ligand to a STING protein, in vitro or in vivo, in the presence of a compound of the application.

In one embodiment, the compounds of the present application are useful as therapeutic agents, and thus may be useful in the treatment of a disease caused by, or associated with, STING expression, activity, and/or function (e.g., deregulation of STING expression, activity, and/or function) or a disease associated with one or more of the intracellular pathways that STING is involved in (e.g. regulation of intracellular DNA-mediated type I interferon activation), such as those described herein.

A ‘selective STING modulator’ can be identified, for example, by comparing the ability of a compound to modulate STING expression/activity/function to its ability to modulate the other proteins or a STING protein from another species. In some embodiments, the selectivity can be identified by measuring the EC₅₀ or IC₅₀ of the compounds.

The compounds of the application are defined herein by their chemical structures and/or chemical names. Where a compound is referred to by both a chemical structure and a chemical name, and the chemical structure and chemical name conflict, the chemical structure is determinative of the compound's identity.

The recitation of a listing of chemical groups in any definition of a variable herein includes definitions of that variable as any single group or combination of listed groups. The recitation of an embodiment for a variable herein includes that embodiment as any single embodiment or in combination with any other embodiments or portions thereof.

In another aspect, the application provides a method of synthesizing a compound disclosed herein. The synthesis of the compounds of the application can be found herein and in the Examples below. Other embodiments are a method of making a compound of any of the formulae herein using any one, or combination of, reactions delineated herein. The method can include the use of one or more intermediates or chemical reagents delineated herein.

The application also provides for a pharmaceutical composition comprising a therapeutically effective amount of a compound of the application, or a pharmaceutically acceptable salt or ester thereof, and a pharmaceutically acceptable carrier.

Another aspect of the present application relates to a kit comprising a compound of the application or a pharmaceutically acceptable salt or ester thereof, or a pharmaceutical composition of the application. In another aspect, the application provides a kit comprising a compound capable of modulating STING activity selected from one or more compounds disclosed herein, or a pharmaceutically acceptable salt or ester thereof, optionally in combination with a second agent and instructions for use.

Another aspect of the present application relates to a compound of the application or a pharmaceutically acceptable salt or ester thereof, or a pharmaceutical composition of the application, for use in the manufacture of a medicament for modulating (e.g., inhibiting or stimulating) a STING protein, for treating or preventing a disease, wherein the diseases is caused by, or associated with, STING expression, activity, and/or function (e.g., deregulation of STING expression, activity, and/or function), or for treating or preventing a disease associated with deregulation of one or more of the intracellular pathways in which a STING protein is involved (e.g., deregulation of intracellular dsDNA mediated type I interferon activation).

Another aspect of the present application relates to use of a compound of the application or a pharmaceutically acceptable salt or ester thereof, or a pharmaceutical composition of the application, in the manufacture of a medicament for modulating (e.g., inhibiting or stimulating) a STING protein, for treating or preventing a disease, wherein the diseases is caused by, or associated with, STING expression, activity, and/or function (e.g., deregulation of STING expression, activity, and/or function), or for treating or preventing a disease associated with deregulation of one or more of the intracellular pathways in which a STING protein is involved (e.g., deregulation of intracellular dsDNA mediated type I interferon activation).

Another aspect of the present application relates to a compound of the application or a pharmaceutically acceptable salt or ester thereof, or a pharmaceutical composition of the application, for use in modulating (e.g., inhibiting or stimulating) a STING protein, in treating or preventing a disease, wherein the diseases is caused by, or associated with, STING expression, activity, and/or function (e.g., deregulation of STING expression, activity, and/or function), or in treating or preventing a disease associated with deregulation of one or more of the intracellular pathways in which a STING protein is involved (e.g. deregulation of intracellular dsDNA mediated type I interferon activation).

Another aspect of the present application relates to use of a compound of the application or a pharmaceutically acceptable salt or ester thereof, or a pharmaceutical composition of the application, in antagonizing a STING protein, in treating or preventing a disease, wherein the diseases is caused by, or associated with, STING expression, activity, and/or function (e.g., deregulation of STING expression, activity, and/or function), or in treating or preventing a disease associated with deregulation of one or more of the intracellular pathways in which a STING protein is involved (e.g., deregulation of intracellular dsDNA mediated type I interferon activation).

### Method of Synthesizing the Compounds

Compounds of the present application can be prepared in a variety of ways using commercially available starting materials, compounds known in the literature, or from readily prepared intermediates, by employing standard synthetic methods and procedures either known to those skilled in the art, or which will be apparent to the skilled artisan in light of the teachings herein. Standard synthetic methods and procedures for the preparation of organic molecules and functional group transformations and manipulations can be obtained from the relevant scientific literature or from standard textbooks in the field. Although not limited to any one or several sources, classic texts such as Smith, M. B., March, J., March's Advanced Organic Chemistry: Reactions, Mechanisms, and Structure, 5^{th} edition, John Wiley & Sons: New York, 2001; and Greene, T. W., Wuts, P. G. M., Protective Groups in Organic Synthesis, 3^{rd} edition, John Wiley & Sons: New York, 1999 are useful and recognized reference textbooks of organic synthesis known to those in the art. The following descriptions of synthetic methods are designed to illustrate, but not to limit, general procedures for the preparation of compounds of the present application. The processes generally provide the desired final compound at or near the end of the overall process, although it may be desirable in certain instances to further convert the compound to a pharmaceutically acceptable salt, ester, or prodrug thereof. Suitable synthetic routes are depicted in the schemes below.

### Synthesis and Evaluation of STAVs

In an embodiment of the present invention, a variety of ssDNA and dsDNA oligonucleotides, containing exonuclease resistant phosphorothioates at the ends (ES) that varied in their nucleotide content were synthesized (clinical grade, TriLink Biotechnologies) using procedures known to a person of ordinary skill in the art. The ssDNA and dsDNA oligonucleotides evaluated to determine which was better at stimulating STING signaling following transfection of normal human and mouse cells including APCs had nucleotide content as follows:

A:T30ES = polyA30ES (SEQ ID NO: 1) +
polyT30ES (SEQ ID NO: 2)
polyA30ES is
(SEQ ID NO: 1)
A(ps)A(ps)A(ps)AAAAAAAAAAAAAAAAAAAAAAAA(ps)A(ps)A
(ps)A,
polyT30ES is
(SEQ ID NO: 2)
T(ps)T(ps)T(ps)TTTTTTTTTTTTTTTTTTTTTTTT(ps)T(ps)T
(ps)T.
A:T50ES = polyA50ES (SEQ ID NO: 3) +
polyT50ES (SEQ ID NO: 4)
polyA50ESis
(SEQ ID NO: 3)
A(ps)A(ps)A(ps)AAAAAAAAAAAAAAAAAAAAAAAAAAAAAAAAAA
AAAAAAAAAA(ps)A(ps)A(ps)A,
polyT50ES is
(SEQ ID NO: 4)
T(ps)T(ps)T(ps)TTTTTTTTTTTTTTTTTTTTTTTTTTTTTTTTTT
TTTTTTTTTT(ps)T(ps)T(ps)T.
A:T60ES = polyA60ES (SEQ ID NO: 5) +
polyT60ES (SEQ ID NO: 6)
polyA60ES is
(SEQ ID NO: 5)
A(ps)A(ps)A(ps)AAAAAAAAAAAAAAAAAAAAAAAAAAAAAAAAAA
AAAAAAAAAAAAAAAAAAAA(ps)A(ps)A(ps)A,
polyT60ES is
(SEQ ID NO: 6)
T(ps)T(ps)T(ps)TTTTTTTTTTTTTTTTTTTTTTTTTTTTTTTTTT
TTTTTTTTTTTTTTTTTTTT(ps)T(ps)T(ps)T.
A:T70ES = polyA70ES (SEQ ID NO: 7) +
polyT70ES (SEQ ID NO: 8)
polyA70ES is
(SEQ ID NO: 7)
A(ps)A(ps)A(ps)AAAAAAAAAAAAAAAAAAAAAAAAAAAAAAAAAA
AAAAAAAAAAAAAAAAAAAAAAAAAAAAAA(ps)A(ps)A(ps)A,
polyT70ES is
(SEQ ID NO: 8)
T(ps)T(ps)T(ps)TTTTTTTTTTTTTTTTTTTTTTTTTTTTTTTTTT
TTTTTTTTTTTTTTTTTTTTTTTTTTTTTT(ps)T(ps)T(ps)T.
A:T80ES = polyA80ES (SEQ ID NO: 9) +
polyT80ES (SEQ ID NO: 10)
polyA80ES is
(SEQ ID NO: 9)
A(ps)A(ps)A(ps)AAAAAAAAAAAAAAAAAAAAAAAAAAAAAAAAAA
AAAAAAAAAAAAAAAAAAAAAAAAAAAAAAAAAAAAAAAA(ps)A(ps)
A(ps)A,
polyT80ES is
(SEQ ID NO: 10)
T(ps)T(ps)T(ps)TTTTTTTTTTTTTTTTTTTTTTTTTTTTTTTTTT
TTTTTTTTTTTTTTTTTTTTTTTTTTTTTTTTTTTTTTTT(PS)T(PS)
T(PS)T.
A:T90ES = polyA90ES (SEQ ID NO: 11) +
polyT90ES (SEQ ID NO: 12)
polyA90ES is
(SEQ ID NO: 11)
A(ps)A(ps)A(ps)AAAAAAAAAAAAAAAAAAAAAAAAAAAAAAAAAA
AAAAAAAAAAAAAAAAAAAAAAAAAAAAAAAAAAAAAAAAAAAAAAAAA
A(ps)A(ps)A(ps)A,
polyT90ES is
(SEQ ID NO: 12)
T(ps)T(ps)T(ps)TTTTTTTTTTTTTTTTTTTTTTTTTTTTTTTTTT
TTTTTTTTTTTTTTTTTTTTTTTTTTTTTTTTTTTTTTTTTTTTTTTTT
T(PS)T(PS)T(Ps)T.
A:T100ES = polyA100ES (SEQ ID NO: 13) +
polyT100ES (SEQ ID NO: 14)
polyA100ES is
(SEQ ID NO: 13)
A(ps)A(ps)A(ps)AAAAAAAAAAAAAAAAAAAAAAAAAAAAAAAAAA
AAAAAAAAAAAAAAAAAAAAAAAAAAAAAAAAAAAAAAAAAAAAAAAAA
AAAAAAAAAAA(ps)A(ps)A(ps)A,
polyT100ES is
(SEQ ID NO: 14)
T(ps)T(ps)T(ps)TTTTTTTTTTTTTTTTTTTTTTTTTTTTTTTTTT
TTTTTTTTTTTTTTTTTTTTTTTTTTTTTTTTTTTTTTTTTTTTTTTTT
TTTTTTTTTTT(PS)T(PS)T(PS)T.
A:T110ES = polyA110ES (SEQ ID NO: 15)
polyT110ES (SEQ ID NO: 16)
polyA110ES is
(SEQ ID NO: 15)
A(ps)A(ps)A(ps)AAAAAAAAAAAAAAAAAAAAAAAAAAAAAAAAAA
AAAAAAAAAAAAAAAAAAAAAAAAAAAAAAAAAAAAAAAAAAAAAAAAA
AAAAAAAAAAAAAAAAAAAAA(ps)A(ps)A(ps)A,
polyT110ES
(SEQ ID NO: 16)
T(ps)T(ps)T(ps)TTTTTTTTTTTTTTTTTTTTTTTTTTTTTTTTTT
TTTTTTTTTTTTTTTTTTTTTTTTTTTTTTTTTTTTTTTTTTTTTTTTT
TTTTTTTTTTTTTTTTTTTTT(PS)T(ps)T(ps)T.
GC30-100ES
(SEQ ID NO: 17)
GC30ES is G(ps)C(ps)G(ps)CGCGCGCGCGCGCGCGCGCGCGCG
(ps)C(ps)G(ps)C.
GC50ES is
(SEQ ID NO: 18)
G(ps)C(ps)G(ps)CGCGCGCGCGCGCGCGCGCGCGCGCGCGCGCGCG
CGCGCGCGCG(ps)C(ps)G(ps)C,
GC60ES is
(SEQ ID NO: 19)
G(ps)C(ps)G(ps)CGCGCGCGCGCGCGCGCGCGCGCGCGCGCGCGCG
CGCGCGCGCGCGCGCGCGCG(ps)C(ps)G(ps)C,
GC70ES is
(SEQ ID NO: 20)
G(ps)C(ps)G(ps)CGCGCGCGCGCGCGCGCGCGCGCGCGCGCGCGCG
CGCGCGCGCGCGCGCGCGCGCGCGCGCGCG(ps)C(ps)G(ps)C,
GC80ES is
(SEQ ID NO: 21)
G(ps)C(ps)G(ps)CGCGCGCGCGCGCGCGCGCGCGCGCGCGCGCGCG
CGCGCGCGCGCGCGCGCGCGCGCGCGCGCGCGCGCGCGCG(ps)C(ps)
G(ps)C,
GC90ES is
(SEQ ID NO: 22)
G(ps)C(ps)G(ps)CGCGCGCGCGCGCGCGCGCGCGCGCGCGCGCGCG
CGCGCGCGCGCGCGCGCGCGCGCGCGCGCGCGCGCGCGCGCGCGCGCGC
G(ps)C(ps)G(ps)C,
and
GC100ES is
(SEQ ID NO: 23)
G(ps)C(ps)G(ps)CGCGCGCGCGCGCGCGCGCGCGCGCGCGCGCGCG
CGCGCGCGCGCGCGCGCGCGCGCGCGCGCGCGCGCGCGCGCGCGCGCGC
GCGCGCGCGCG(ps)C(ps)G(ps)C.
STAV1 = polyA76ES (SEQ ID NO: 24) +
polyT76ES (SEQ ID NO: 25)
PolyA76ES is
(SEQ ID NO: 24)
A(ps)A(ps)A(ps)AAAAAAAAAAAAAAAAAAAAAAAAAAAAAAAAAA
AAAAAAAAAAAAAAAAAAAAAAAAAAAAAAAAAAAA(ps)A(ps)A
(ps)A,
PolyT76ES
(SEQ ID NO: 25)
T(ps)T(ps)T(ps)TTTTTTTTTTTTTTTTTTTTTTTTTTTTTTTTTT
TTTTTTTTTTTTTTTTTTTTTTTTTTTTTTTTTTTT(ps)T(ps)T
(ps)T.
STAV2 = polyAC76ES (SEQ ID NO: 26)
polyTG76ES (SEQ ID NO: 27)
PolyAC76ES is
(SEQ ID NO: 26)
A(ps)C(ps)A(ps)CACACACACACACACACACACACACACACACACA
CACACACACACACACACACACACACACACACACACA(ps)C(ps)A
(ps)C,
PolyTG76ES
(SEQ ID NO: 27)
G(ps)T(ps)G(ps)TGTGTGTGTGTGTGTGTGTGTGTGTGTGTGTGTG
TGTGTGTGTGTGTGTGTGTGTGTGTGTGTGTGTGTG(ps)T(ps)G
(ps)T.
PolyXX76ES
PolyAT76ES is
(SEQ ID NO: 28)
A(ps)T(ps)A(ps)TATATATATATATATATATATATATATATATATA
TATATATATATATATATATATATATATATATATATA(ps)T(ps)A
(ps)T,
PolyTA76ES is
(SEQ ID NO: 29)
T(ps)A(ps)T(ps)ATATATATATATATATATATATATATATATATAT
ATATATATATATATATATATATATATATATATATAT(ps)A(ps)T
(ps)A,
PolyACTG76ES is
(SEQ ID NO: 30)
A(ps)C(ps)T(ps)GACTGACTGACTGACTGACTGACTGACTGACTGA
CTGACTGACTGACTGACTGACTGACTGACTGACTGA(ps)C(ps)T
(ps)G,
PolyCAGT76ES is
(SEQ ID NO: 31)
C(ps)A(ps)G(ps)TCAGTCAGTCAGTCAGTCAGTCAGTCAGTCAGTC
AGTCAGTCAGTCAGTCAGTCAGTCAGTCAGTCAGTC(ps)A(ps)G
(ps)T,
HSVRL2 intron-S is
(SEQ ID NO: 32)
G(ps)A(ps)C(ps)CCTATCGATACAGGGCACGGGGTCGAACTGTTGG
GTTTCGCCATGGTACCCCCTGCATTTATATAGCCAG(ps)A(ps)C
(ps)C,
HSVRL2 intron-AS is
(SEQ ID NO: 33)
G(ps)G(ps)T(ps)CTGGCTATATAAATGCAGGGGGTACCATGGCGAA
ACCCAACAGTTCGACCCCGTGCCCTGTATCGATAGG(ps)G(ps)T
(ps)C.
PolyX90ES
polyA90ES-FAMisFAM-
(SEQ ID NO: 35)
A(ps)A(ps)A(ps)AAAAAAAAAAAAAAAAAAAAAAAAAAAAAAAAAA
AAAAAAAAAAAAAAAAAAAAAAAAAAAAAAAAAAAAAAAAAAAAAAAAA
A(ps)A(ps)A(ps)A,
polyT90ES is
FAM-
(SEQ ID NO: 36)
T(ps)T(ps)T(ps)TTTTTTTTTTTTTTTTTTTTTTTTTTTTTTTTTT
TTTTTTTTTTTTTTTTTTTTTTTTTTTTTTTTTTTTTTTTTTTTTTTTT
T(PS)T(PS)T(Ps)T.
STAV3 = polyAT76ES (SEQ ID NO: 37)
polyTA76ES (SEQ ID NO: 38)
PolyAT:TA76ES is
(SEQ ID NO: 37)
A(ps)T(ps)T(ps)AATTAATTAATTAATTAATTAATTAATTAATTAA
TTAATTAATTAATTAATTAATTAATTAATTAATTAA(ps)T(ps)T
(ps)A,
PolyTA:AT76ES
(SEQ ID NO: 38)
T(ps)A(ps)A(ps)TTAATTAATTAATTAATTAATTAATTAATTAATT
AATTAATTAATTAATTAATTAATTAATTAATTAATT(ps)A(ps)A
(ps)T.
STAV4 = (SEQ ID NO: 39) +
(SEQ ID NO: 40)
(SEQ ID NO: 39)
A(ps)C(ps)T(ps)GACTGACTGACTGACTGACTGACTGACTGACTGA
CTGACTGACTGACTGACTGACTGACTGACTGACTGA(ps)C(ps)T
(ps)G,
(SEQ ID NO: 40)
C(ps)A(ps)G(ps)TCAGTCAGTCAGTCAGTCAGTCAGTCAGTCAGTC
AGTCAGTCAGTCAGTCAGTCAGTCAGTCAGTCAGTC(ps)A(ps)G
(ps)T.
STAV5 = (SEQ ID NO: 41) +
(SEQ ID NO: 42)
(SEQ ID NO: 41)
G(ps)A(ps)C(ps)CCTATCGATACAGGGCACGGGGTCGAACTGTTGG
GTTTCGCCATGGTACCCCCTGCATTTATATAGCCAG(ps)A(ps)C
(ps)C,
(SEQ ID NO: 42)
G(ps)G(ps)T(ps)CTGGCTATATAAATGCAGGGGGTACCATGGCGAA
ACCCAACAGTTCGACCCCGTGCCCTGTATCGATAGG(ps)G(ps)T
(ps)C.
STAV6 = (SEQ ID NO: 43) +
(SEQ ID NO: 44)
(SEQ ID NO: 43)
A(ps)A(ps)A(ps)AAAAAAAAAAAAAAAAAAAAAAAAAAAAAAAAAA
AAAAAAAAAAAAAAAAAAAAAAAAAAAAAAAAAAAAAAAAAAAAAAAAA
A(ps)A(ps)A(ps)A,
(SEQ ID NO: 44)
T(ps)T(ps)T(ps)TTTTTTTTTTTTTTTTTTTTTTTTTTTTTTTTTT
TTTTTTTTTTTTTTTTTTTTTTTTTTTTTTTTTTTTTTTTTTTTTTTTT
T(PS)T(PS)T(PS)T.
STAV7 = (SEQ ID NO: 45) +
(SEQ ID NO: 46)
(SEQ ID NO: 45)
G(ps)C(ps)G(ps)CGCGCGCGCGCGCGCGCGCGCGCGCGCGCGCGCG
CGCGCGCGCGCGCGCGCGCGCGCGCGCGCGCGCGCGCGCGCGCGCGCGC
G(ps)C(ps)G(ps)C,
(SEQ ID NO: 46)
C(ps)G(ps)C(ps)GCGCGCGCGCGCGCGCGCGCGCGCGCGCGCGCGC
GCGCGCGCGCGCGCGCGCGCGCGCGCGCGCGCGCGCGCGCGCGCGCGCG
C(ps)G(ps)C(ps)G.

A number of STAVs that were greater than 70 bp were effective in stimulating STING-based cytokine production. Based on the result that STAVs greater than 70 bp were effective in stimulating STING, three (3) STAVs were used herein as follows: STAV1 a double-stranded polyA:T76ES, oligonucleotides (SEQ ID NO:24)+(SEQ ID NO:25); STAV2 a double-stranded polyAC:TG76ES, oligonucleotides (SEQ ID NO:26)+(SEQ ID NO:27); and STAV3 a double-stranded polyAT:TA76ES, oligonucleotides (SEQ ID NO:37)+(SEQ ID NO:38).

### Nano-STAV Synthesis

In an embodiment of the present invention, a LNP can be synthesized from distearoylphosphatidylcholine, cholesterol, MC3, and DMG-PEG 2000 by dissolving in ethanol that is rapidly mixed with the STAV1 (SEQ ID NO:24)+(SEQ ID NO:25); STAV2 (SEQ ID NO:26)+(SEQ ID NO:27); or STAV3 (SEQ ID NO:37)+(SEQ ID NO:38) in aqueous buffer at a pH approximately 4. The resulting dispersion can then be dialyzed against a normal saline buffer to remove residual ethanol and raise the pH above approximately 7.4, (where approximately means+−pH 1) to produce the finished Nano-STAV1, Nano-STAV2, and Nano-STAV3 respectively.

### Check Point Inhibitor Analysis

Anti-PD-L1 (IgG BE0091 or anti-PD-L1 BE0101, BioXcell) and anti-PD1 (J43 BE0033-2, BioXcell) were used in the B16 melanoma model. Sex matched C57/BL6 mice (n=10) were inoculated with B16-OVA (5×10⁵) on the flanks. After 7, 10, and 13 days, when tumors are 50 mm³ in volume, 25 μl (4 μg/mL; 0.1 μg/mouse) of Nano-STAVs (STAV1 a double-stranded polyA:T76ES, oligonucleotides (SEQ ID NO:24)+(SEQ ID NO:25); STAV2 a double-stranded polyAC:TG76ES, oligonucleotides (SEQ ID NO:26)+(SEQ ID NO:27); and STAV3 a double-stranded polyAT:TA76ES, oligonucleotides (SEQ ID NO:37)+(SEQ ID NO:38)) were injected i.t. in presence or absence of anti-PD-1 or anti-PD-L1 (50 μg/mouse).

The compounds of the present application can be prepared in a number of ways well known to those skilled in the art of organic synthesis. By way of example, compounds of the present application can be synthesized using the methods described below, together with synthetic methods known in the art of synthetic organic chemistry, or variations thereon as appreciated by those skilled in the art. Preferred methods include but are not limited to those methods described below.

Compounds of the present application can be synthesized by following the steps outlined in the following Schemes, which comprise different sequences of assembling intermediates. Starting materials are either commercially available or made by known procedures in the reported literature or as illustrated.

A compound of the application can be prepared as a pharmaceutically acceptable acid addition salt by reacting the free base form of the compound with a pharmaceutically acceptable inorganic or organic acid. Alternatively, a pharmaceutically acceptable base addition salt of a compound of the application can be prepared by reacting the free acid form of the compound with a pharmaceutically acceptable inorganic or organic base. The pharmaceutically acceptable salt may include various counterions, e.g., counterions of the inorganic or organic acid, counterions of the inorganic or organic base, or counterions afforded by counterion exchange.

Acids and bases useful in the methods herein are known in the art. Acid catalysts are any acidic chemical, which can be inorganic (e.g., hydrochloric, sulfuric, nitric acids, aluminum trichloride) or organic (e.g., camphorsulfonic acid, p-toluenesulfonic acid, acetic acid, ytterbium triflate) in nature. Acids are useful in either catalytic or stoichiometric amounts to facilitate chemical reactions. Bases are any basic chemical, which can be inorganic (e.g., sodium bicarbonate, potassium hydroxide) or organic (e.g., triethylamine, pyridine) in nature. Bases are useful in either catalytic or stoichiometric amounts to facilitate chemical reactions.

Alternatively, the salt forms of the compounds of the application can be prepared using salts of the starting materials or intermediates. The free acid or free base forms of the compounds of the application can be prepared from the corresponding base addition salt or acid addition salt from, respectively. For example, a compound of the application in an acid addition salt form can be converted to the corresponding free base by treating with a suitable base (e.g., ammonium hydroxide solution, sodium hydroxide, and the like). A compound of the application in a base addition salt form can be converted to the corresponding free acid by treating with a suitable acid (e.g., hydrochloric acid, etc.).

Those skilled in the art will recognize if a stereo center exists in the compounds disclosed herein. Accordingly, the present application includes both possible stereoisomers (unless specified in the synthesis) and includes not only racemic compounds but the individual enantiomers and/or diastereomers as well. When a compound is desired as a single enantiomer or diastereomer, it may be obtained by stereospecific synthesis or by resolution of the final product or any convenient intermediate. Resolution of the final product, an intermediate, or a starting material may be affected by any suitable method known in the art. See, for example, “Stereochemistry of Organic Compounds” by E. L. Eliel, S. H. Wilen, and L. N. Mander (Wiley-Interscience, 1994).

Compounds of the present application that contain non pyrrolo quinoxaline nitrogens can be converted to N-oxides by treatment with an oxidizing agent (e.g., 3-chloroperoxybenzoic acid (m-CPBA) and/or hydrogen peroxides) to afford other compounds of the present application. Thus, all shown and claimed non pyrrolo quinoxaline nitrogen-containing compounds are considered, when allowed by valency and structure, to include both the compound as shown and its N-oxide derivative (which can be designated as N→O or N⁺—O—). Furthermore, in other instances, the nitrogens in the non pyrrolo quinoxaline compounds of the present application can be converted to N-hydroxy or N-alkoxy compounds. For example, N-hydroxy compounds can be prepared by oxidation of the parent amine by an oxidizing agent such as m-CPBA. All shown and claimed non pyrrolo quinoxaline nitrogen-containing compounds are also considered, when allowed by valency and structure, to cover both the compound as shown and its N-hydroxy (i.e., N—OH) and N-alkoxy (i.e., N—OR, wherein R is substituted or unsubstituted C₁-C₆ alkyl, C₁-C₆ alkenyl, C₁-C₆ alkynyl, 3-14-membered carbocycle or 3-14-membered heterocycle) derivatives.

Prodrugs of the compounds of the application can be prepared by methods known to those of ordinary skill in the art (e.g., for further details see Saulnier et al., (1994), Bioorganic and Medicinal Chemistry Letters, Vol. 4, p. 1985, which is herein expressly incorporated by reference in its entirety and for all purposes). For example, appropriate prodrugs can be prepared by reacting a non-derivatized compound of the application with a suitable carbamylating agent (e.g., 1,1-acyloxyalkylcarbanochloridate, para-nitrophenyl carbonate, or the like). Specifically, the central N-acetic acid moiety, and other analogous carboxylic acid groups, of the compounds of the present invention can be modified through techniques known in the art to produce effective prodrugs of the present invention.

Protected derivatives of the compounds of the application can be made by means known to those of ordinary skill in the art. A detailed description of techniques applicable to the creation of protecting groups and their removal can be found in T. W. Greene, “Protecting Groups in Organic Chemistry”, 3rd edition, John Wiley and Sons, Inc., 1999, which is herein expressly incorporated by reference in its entirety and for all purposes.

Compounds of the present application can be conveniently prepared, or formed during the process of the application, as solvates (e.g., hydrates). Hydrates of compounds of the present application can be conveniently prepared by recrystallization from an aqueous/organic solvent mixture, using organic solvents such as dioxin, tetrahydrofuran or methanol.

Optical isomers may be prepared from their respective optically active precursors by the procedures described herein, or by resolving the racemic mixtures. The resolution can be carried out in the presence of a resolving agent, by chromatography or by repeated crystallization or by some combination of these techniques which are known to those skilled in the art. Further details regarding resolutions can be found in Jacques, et al., Enantiomers, Racemates, and Resolutions (John Wiley & Sons, 1981), which is herein expressly incorporated by reference in its entirety and for all purposes

The synthesized compounds can be separated from a reaction mixture and further purified by a method such as column chromatography, high pressure liquid chromatography, or recrystallization. As can be appreciated by the skilled artisan, further methods of synthesizing the compounds of the formulae herein will be evident to those of ordinary skill in the art. Additionally, the various synthetic steps may be performed in an alternate sequence or order to give the desired compounds. In addition, the solvents, temperatures, reaction durations, etc. delineated herein are for purposes of illustration only and one of ordinary skill in the art will recognize that variation of the reaction conditions can produce the desired bridged macrocyclic products of the present application. Synthetic chemistry transformations and protecting group methodologies (protection and deprotection) useful in synthesizing the compounds described herein are known in the art and include, for example, those such as described in R. Larock, Comprehensive Organic Transformations, VCH Publishers (1989); T. W. Greene and P. G. M. Wuts, Protective Groups in Organic Synthesis, 2d. Ed., John Wiley and Sons (1991); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), which are herein expressly incorporated by reference in their entireties and for all purposes, and subsequent editions thereof.

In one aspect, the present application provides a method of inhibiting a STING protein. The method comprises administering to a subject in need thereof an effective amount of a compound of the application or a pharmaceutically acceptable salt or ester thereof, or a pharmaceutical composition of the application.

In some embodiments, the modulation of a STING protein activity is measured by IC₅₀. In some embodiments, the modulation of a STING protein activity is measured by EC₅₀.

A compound of the present application (e.g., a compound of any of the formulae described herein, or selected from any compounds described herein) is capable of treating or preventing a disease, wherein the diseases is caused by, or associated with, STING expression, activity, and/or function (e.g., deregulation of STING expression, activity, and/or function) or a disease associated with deregulation of one or more of the intracellular pathways in which a STING protein is involved (e.g., deregulation of intracellular dsDNA mediated type I interferon activation).

In one aspect, the present application provides a method of treating or preventing a disease, wherein the diseases is caused by, or associated with, STING expression, activity, and/or function (e.g., deregulation of STING expression, activity, and/or function). The method comprises administering to a subject in need thereof an effective amount of a STING antagonist compound of the application or a pharmaceutically acceptable salt or ester thereof, or a pharmaceutical composition of the application. In one aspect, the disease is a STING mediated disorder.

In one aspect, the present application provides a method of treating or preventing a disease associated with deregulation of one or more of the intracellular pathways in which a STING protein is involved (e.g., deregulation of intracellular dsDNA mediated type I interferon activation). The method comprises administering to a subject in need thereof an effective amount of a STING antagonist compound of the application or a pharmaceutically acceptable salt or ester thereof, or a pharmaceutical composition of the application.

In one embodiment, the present application provides a method of treating or preventing any of the diseases, disorders, and conditions described herein, wherein the subject is a human. In one embodiment, the application provides a method of treating. In one embodiment, the application provides a method of preventing.

As antagonists of a STING protein, the compounds and compositions of this application are particularly useful for treating or lessening the severity of a disease, condition, or disorder where a STING protein or one or more of the intracellular pathways that STING is involved is implicated in the disease, condition, or disorder. In one embodiment, the present application provides a method for treating or lessening the severity of a disease, condition, or disorder with STING antagonist compounds that modulate binding of a cyclic di-nucleotide, (CDN) including non-canonical cyclic di-nucleotide, such as 2′3′cGAMP, to a STING protein. In one embodiment, the present application provides a method for treating or lessening the severity of a disease, condition, or disorder with compounds that modulate the synthesis of type I interferon and/or type I IFN response and other cytokines, chemokines (STING-inducible proteins).

In one aspect, the present application also provides a method of treating or preventing cell proliferative disorders such as hyperplasias, dysplasias, or pre-cancerous lesions. Dysplasia is the earliest form of pre-cancerous lesion recognizable in a biopsy by a pathologist. The compounds of the present application may be administered for the purpose of preventing hyperplasias, dysplasias, or pre-cancerous lesions from continuing to expand or from becoming cancerous. Examples of pre-cancerous lesions may occur in skin, esophageal tissue, breast, and cervical intra-epithelial tissue.

In one embodiment, the disease or disorder includes, but is not limited to, immune disorders, autoimmunity, a cell proliferative disease or disorder, cancer, inflammation, graft vs host, transplantation, gastrointestinal disorder, rheumatoid arthritis, systemic lupus, cachexia, neurodegenerative disease or disorders, neurological diseases or disorders, cardiac dysfunction, or microbial infection (e.g., viral, bacterial, and/or fungi infection, parasitic, or infection caused by other microorganism).

In one embodiment, the disease or disorder is a cell proliferative disease or disorder.

As used herein, the term ‘cell proliferative disorder’ refers to conditions in which unregulated or abnormal growth, or both, of cells can lead to the development of an unwanted condition or disease, which may or may not be cancerous. The term ‘rapidly dividing cell’ as used herein is defined as any cell that divides at a rate that exceeds or is greater than what is expected or observed among neighboring or juxtaposed cells within the same tissue. A cell proliferative disease or disorder includes a precancer or a precancerous condition. A cell proliferative disease or disorder includes cancer.

In one embodiment, the proliferative disease or disorder is non-cancerous. In one embodiment, the non-cancerous disease or disorder includes, but is not limited to, rheumatoid arthritis; inflammation; autoimmune disease; lymphoproliferative conditions; acromegaly; rheumatoid spondylitis; osteoarthritis; gout; other arthritic conditions; sepsis; septic shock; endotoxic shock; gram-negative sepsis; toxic shock syndrome; asthma; adult respiratory distress syndrome; chronic obstructive pulmonary disease; chronic pulmonary inflammation; inflammatory bowel disease; Crohn's disease; skin-related hyperproliferative disorders; psoriasis; eczema; atopic dermatitis; hyperpigmentation disorders; eye-related hyperproliferative disorders; age-related macular degeneration; ulcerative colitis; pancreatic fibrosis; hepatic fibrosis; acute and chronic renal disease; irritable bowel syndrome; pyresis; restenosis; cerebral malaria; stroke and ischemic injury; neural trauma; Alzheimer's disease; Huntington's disease; Parkinson's disease; acute and chronic pain; allergic rhinitis; allergic conjunctivitis; chronic heart failure; acute coronary syndrome; cachexia; malaria; leprosy; leishmaniasis; Lyme disease; Reiter's syndrome; acute synovitis; muscle degeneration, bursitis; tendonitis; tenosynovitis; herniated, ruptures, or prolapsed intervertebral disk syndrome; osteopetrosis; thrombosis; restenosis; silicosis; pulmonary sarcosis; bone resorption diseases, such as osteoporosis; graft-versus-host reaction; fibroadipose hyperplasia; spinocerebullar ataxia type 1; CLOVES syndrome; Harlequin ichthyosis; macrodactyly syndrome; Proteus syndrome (Wiedemann syndrome); LEOPARD syndrome; systemic sclerosis; Multiple Sclerosis; lupus; fibromyalgia; AIDS and other viral diseases such as Herpes Zoster, Herpes Simplex I or II, influenza virus and cytomegalovirus; diabetes mellitus; hemihyperplasia-multiple lipomatosis syndrome; megalencephaly; rare hypoglycemia, Klippel-Trenaunay syndrome; harmatoma; Cowden syndrome; or overgrowth-hyperglycemia.

In one embodiment, the proliferative disease or disorder is cancer. In one embodiment, the cancer is lung cancer, colon cancer, breast cancer, prostate cancer, liver cancer, pancreas cancer, brain cancer, kidney cancer, ovarian cancer, stomach cancer, skin cancer, bone cancer, gastric cancer, breast cancer, pancreatic cancer, glioma, glioblastoma, hepatocellular carcinoma, papillary renal carcinoma, head and neck squamous cell carcinoma, leukemias, lymphomas, myelomas, or solid tumors.

The term ‘cancer’ includes, but is not limited to, the following cancers: breast; ovary; cervix; prostate; testis, genitourinary tract; esophagus; larynx, glioblastoma; neuroblastoma; stomach; skin, keratoacanthoma; lung, epidermoid carcinoma, large cell carcinoma, small cell carcinoma, lung adenocarcinoma; bone; colon; colorectal; adenoma; pancreas, adenocarcinoma; thyroid, follicular carcinoma, undifferentiated carcinoma, papillary carcinoma; seminoma; melanoma; sarcoma; bladder carcinoma; liver carcinoma and biliary passages; kidney carcinoma; myeloid disorders; lymphoid disorders, Hodgkin's, hairy cells; buccal cavity and pharynx (oral), lip, tongue, mouth, pharynx; small intestine; colon, rectum, large intestine, rectum, brain and central nervous system; chronic myeloid leukemia (CML), and leukemia. The term ‘cancer’ includes, but is not limited to, the following cancers: myeloma, lymphoma, or a cancer selected from gastric, renal, or and the following cancers: head and neck, oropharangeal, non-small cell lung cancer (NSCLC), endometrial, hepatocarcinoma, Non-Hodgkins lymphoma, and pulmonary.

The term ‘cancer’ also refers to any cancer caused by the proliferation of malignant neoplastic cells, such as tumors, neoplasms, carcinomas, sarcomas, leukemias, lymphomas and the like. For example, cancers include, but are not limited to, mesothelioma, leukemias and lymphomas such as cutaneous T-cell lymphomas (CTCL), noncutaneous peripheral T-cell lymphomas, lymphomas associated with human T-cell lymphotrophic virus (HTLV) such as adult T-cell leukemia/lymphoma (ATLL), B-cell lymphoma, acute nonlymphocytic leukemias, chronic lymphocytic leukemia, chronic myelogenous leukemia, acute myelogenous leukemia, lymphomas, and multiple myeloma, non-Hodgkin lymphoma, acute lymphatic leukemia (ALL), chronic lymphatic leukemia (CLL), Hodgkin's lymphoma, Burkitt lymphoma, adult T-cell leukemia lymphoma, acute-myeloid leukemia (AML), chronic myeloid leukemia (CML), or hepatocellular carcinoma. Further examples include myelodisplastic syndrome, childhood solid tumors such as brain tumors, neuroblastoma, retinoblastoma, Wilms' tumor, bone tumors, and soft-tissue sarcomas, common solid tumors of adults such as head and neck cancers (e.g., oral, laryngeal, nasopharyngeal and esophageal), genitourinary cancers (e.g., prostate, bladder, renal, uterine, ovarian, testicular), lung cancer (e.g., small-cell and non-small cell), breast cancer, pancreatic cancer, melanoma and other skin cancers, stomach cancer, brain tumors, tumors related to Gorlin's syndrome (e.g., medulloblastoma, meningioma, etc.), and liver cancer. Additional exemplary forms of cancer which may be treated by the subject compounds include, but are not limited to, cancer of skeletal or smooth muscle, stomach cancer, cancer of the small intestine, rectum carcinoma, cancer of the salivary gland, endometrial cancer, adrenal cancer, anal cancer, rectal cancer, parathyroid cancer, and pituitary cancer.

Cancer may also include colon carcinoma, familial adenomatous polyposis carcinoma and hereditary non-polyposis colorectal cancer, or melanoma. Further, cancers include, but are not limited to, labial carcinoma, larynx carcinoma, hypopharynx carcinoma, tongue carcinoma, salivary gland carcinoma, gastric carcinoma, adenocarcinoma, thyroid cancer (medullary and papillary thyroid carcinoma), renal carcinoma, kidney parenchyma carcinoma, cervix carcinoma, uterine corpus carcinoma, endometrium carcinoma, chorion carcinoma, testis carcinoma, urinary carcinoma, melanoma, brain tumors such as glioblastoma, astrocytoma, meningioma, medulloblastoma and peripheral neuroectodermal tumors, gall bladder carcinoma, bronchial carcinoma, multiple myeloma, basalioma, teratoma, retinoblastoma, choroidea melanoma, seminoma, rhabdomyosarcoma, craniopharyngeoma, osteosarcoma, chondrosarcoma, myosarcoma, liposarcoma, fibrosarcoma, Ewing sarcoma, and plasmocytoma.

Cancer may also include colorectal, thyroid, breast, and lung cancer; and myeloproliferative disorders, such as polycythemia vera, thrombocythemia, myeloid metaplasia with myelofibrosis, chronic myelogenous leukemia, chronic myelomonocytic leukemia, hypereosinophilic syndrome, juvenile myelomonocytic leukemia, and systemic mast cell disease. In one embodiment, the compounds of this application are useful for treating hematopoietic disorders, in particular, acute-myelogenous leukemia (AML), chronic-myelogenous leukemia (CML), acute-promyelocytic leukemia, and acute lymphocytic leukemia (ALL).

Exemplary cancers may also include, but are not limited to, adrenocortical carcinoma, AIDS-related cancers, AIDS-related lymphoma, anal cancer, anorectal cancer, cancer of the anal canal, appendix cancer, childhood cerebellar astrocytoma, childhood cerebral astrocytoma, basal cell carcinoma, skin cancer (non-melanoma), biliary cancer, extrahepatic bile duct cancer, intrahepatic bile duct cancer, bladder cancer, uringary bladder cancer, bone and joint cancer, osteosarcoma and malignant fibrous histiocytoma, brain cancer, brain tumor, brain stem glioma, cerebellar astrocytoma, cerebral astrocytoma/malignant glioma, ependymoma, medulloblastoma, supratentorial primitive neuroectodeimal tumors, visual pathway and hypothalamic glioma, breast cancer, bronchial adenomas/carcinoids, carcinoid tumor, gastrointestinal, nervous system cancer, nervous system lymphoma, central nervous system cancer, central nervous system lymphoma, cervical cancer, childhood cancers, chronic lymphocytic leukemia, chronic myelogenous leukemia, chronic myeloproliferative disorders, colon cancer, colorectal cancer, cutaneous T-cell lymphoma, lymphoid neoplasm, mycosis fungoides, Seziary Syndrome, endometrial cancer, esophageal cancer, extracranial germ cell tumor, extragonadal germ cell tumor, extrahepatic bile duct cancer, eye cancer, intraocular melanoma, retinoblastoma, gallbladder cancer, gastric (stomach) cancer, gastrointestinal carcinoid tumor, gastrointestinal stromal tumor (GIST), germ cell tumor, ovarian germ cell tumor, gestational trophoblastic tumor glioma, head and neck cancer, hepatocellular (liver) cancer, Hodgkin lymphoma, hypopharyngeal cancer, intraocular melanoma, ocular cancer, islet cell tumors (endocrine pancreas), Kaposi Sarcoma, kidney cancer, renal cancer, kidney cancer, laryngeal cancer, acute lymphoblastic leukemia, acute myeloid leukemia, chronic lymphocytic leukemia, chronic myelogenous leukemia, hairy cell leukemia, lip and oral cavity cancer, liver cancer, lung cancer, non-small cell lung cancer, small cell lung cancer, AIDS-related lymphoma, non-Hodgkin lymphoma, primary central nervous system lymphoma, Waldenstram macroglobulinemia, medulloblastoma, melanoma, intraocular (eye) melanoma, Merkel cell carcinoma, mesothelioma malignant, mesothelioma, metastatic squamous neck cancer, mouth cancer, cancer of the tongue, multiple endocrine neoplasia syndrome, Mycosis fungoides, myelodysplastic syndromes, myelodysplastic/myeloproliferative diseases, chronic myelogenous leukemia, acute myeloid leukemia, multiple myeloma, chronic myeloproliferative disorders, nasopharyngeal cancer, neuroblastoma, oral cancer, oral cavity cancer, oropharyngeal cancer, ovarian cancer, ovarian epithelial cancer, ovarian low malignant potential tumor, pancreatic cancer, islet cell pancreatic cancer, paranasal sinus and nasal cavity cancer, parathyroid cancer, penile cancer, pharyngeal cancer, pheochromocytoma, pineoblastoma and supratentorial primitive neuroectodermal tumors, pituitary tumor, plasma cell neoplasm/multiple myeloma, pleuropulmonary blastoma, prostate cancer, rectal cancer, renal pelvis and ureter, transitional cell cancer, retinoblastoma, rhabdomyosarcoma, salivary gland cancer, Ewing family of sarcoma tumors, Kaposi Sarcoma, soft tissue sarcoma, uterine cancer, uterine sarcoma, skin cancer (non-melanoma), skin cancer (melanoma), merkel cell skin carcinoma, small intestine cancer, soft tissue sarcoma, squamous cell carcinoma, stomach (gastric) cancer, supratentorial primitive neuroectodermal tumors, testicular cancer, throat cancer, thymoma, thymoma and thymic carcinoma, thyroid cancer, transitional cell cancer of the renal pelvis and ureter and other urinary organs, gestational trophoblastic tumor, urethral cancer, endometrial uterine cancer, uterine sarcoma, uterine corpus cancer, vaginal cancer, vulvar cancer, and Wilm's Tumor.

A ‘cell proliferative disorder of the hematologic system’ is a cell proliferative disease or disorder involving cells of the hematologic system. A cell proliferative disorder of the hematologic system can include lymphoma, leukemia, myeloid neoplasms, mast cell neoplasms, myelodysplasia, benign monoclonal gammopathy, lymphomatoid granulomatosis, lymphomatoid papulosis, polycythemia vera, chronic myelocytic leukemia, agnogenic myeloid metaplasia, and essential thrombocythemia. A cell proliferative disorder of the hematologic system can include hyperplasia, dysplasia, and metaplasia of cells of the hematologic system. Compounds and compositions of the present application may be used to treat a cancer selected from the group consisting of a hematologic cancer or a hematologic cell proliferative disorder. A hematologic cancer can include multiple myeloma, lymphoma (including Hodgkin's lymphoma, non-Hodgkin's lymphoma, childhood lymphomas, and lymphomas of lymphocytic and cutaneous origin), leukemia (including childhood leukemia, hairy-cell leukemia, acute lymphocytic leukemia, acute myelocytic leukemia, chronic lymphocytic leukemia, chronic myelocytic leukemia, chronic myelogenous leukemia, and mast cell leukemia), myeloid neoplasms, and mast cell neoplasms.

A ‘cell proliferative disorder of the lung’ is a cell proliferative disease or disorder involving cells of the lung. Cell proliferative disorders of the lung can include all forms of cell proliferative disorders affecting lung cells. Cell proliferative disorders of the lung can include lung cancer, a precancer or precancerous condition of the lung, benign growths or lesions of the lung, and malignant growths or lesions of the lung, and metastatic lesions in tissue and organs in the body other than the lung. Compounds and compositions of the present application may be used to treat lung cancer or cell proliferative disorders of the lung. Lung cancer can include all forms of cancer of the lung. Lung cancer can include malignant lung neoplasms, carcinoma in situ, typical carcinoid tumors, and atypical carcinoid tumors. Lung cancer can include small cell lung cancer (SCLC), non-small cell lung cancer (NSCLC), squamous cell carcinoma, adenocarcinoma, small cell carcinoma, large cell carcinoma, adenosquamous cell carcinoma, and mesothelioma. Lung cancer can include ‘scar carcinoma’, bronchioalveolar carcinoma, giant cell carcinoma, spindle cell carcinoma, and large cell neuroendocrine carcinoma. Lung cancer can include lung neoplasms having histologic and ultrastructual heterogeneity (e.g., mixed cell types).

Cell proliferative disorders of the lung can also include hyperplasia, metaplasia, and dysplasia of the lung. Cell proliferative disorders of the lung can include asbestos-induced hyperplasia, squamous metaplasia, and benign reactive mesothelial metaplasia. Cell proliferative disorders of the lung can include replacement of columnar epithelium with stratified squamous epithelium, and mucosal dysplasia. Individuals exposed to inhaled injurious environmental agents such as cigarette smoke and asbestos may be at increased risk for developing cell proliferative disorders of the lung. Prior lung diseases that may predispose individuals to development of cell proliferative disorders of the lung can include chronic interstitial lung disease, necrotizing pulmonary disease, scleroderma, rheumatoid disease, sarcoidosis, interstitial pneumonitis, tuberculosis, repeated pneumonias, idiopathic pulmonary fibrosis, granulomata, asbestosis, fibrosing alveolitis, and Hodgkin's disease.

A ‘cell proliferative disorder of the colon’ is a cell proliferative disorder involving cells of the colon. A cell proliferative disorder of the colon includes colon cancer. Compounds and compositions of the present application may be used to treat colon cancer or cell proliferative disorders of the colon. Colon cancer can include all forms of cancer of the colon. Colon cancer can include sporadic and hereditary colon cancers. Colon cancer can include malignant colon neoplasms, carcinoma in situ, typical carcinoid tumors, and atypical carcinoid tumors. Colon cancer can include adenocarcinoma, squamous cell carcinoma, and adenosquamous cell carcinoma. Colon cancer can be associated with a hereditary syndrome selected from the group consisting of hereditary nonpolyposis colorectal cancer, familial adenomatous polyposis, Gardner's syndrome, Peutz-Jeghers syndrome, Turcot's syndrome and juvenile polyposis. Colon cancer can be caused by a hereditary syndrome selected from the group consisting of hereditary nonpolyposis colorectal cancer, familial adenomatous polyposis, Gardner's syndrome, Peutz-Jeghers syndrome, Turcot's syndrome, and juvenile polyposis.

Cell proliferative disorders of the colon can also include colon cancer, precancerous conditions of the colon, adenomatous polyps of the colon and metachronous lesions of the colon. A cell proliferative disorder of the colon can include adenoma. Cell proliferative disorders of the colon can be characterized by hyperplasia, metaplasia, and dysplasia of the colon. Prior colon diseases that may predispose individuals to development of cell proliferative disorders of the colon can include prior colon cancer. Current disease that may predispose individuals to development of cell proliferative disorders of the colon can include Crohn's disease and ulcerative colitis. A cell proliferative disorder of the colon can be associated with a mutation in a gene selected from the group consisting of p53, ras, FAP and DCC. An individual can have an elevated risk of developing a cell proliferative disorder of the colon due to the presence of a mutation in a gene selected from the group consisting of p53, ras, FAP and DCC.

A ‘cell proliferative disorder of the pancreas’ is a cell proliferative disorder involving cells of the pancreas. Compounds and compositions of the present application may be used to treat pancreatic cancer or cell proliferative disorders of the pancreas. Cell proliferative disorders of the pancreas can include all forms of cell proliferative disorders affecting pancreatic cells. Cell proliferative disorders of the pancreas can include pancreas cancer, a precancer or precancerous condition of the pancreas, hyperplasia of the pancreas, and dysaplasia of the pancreas, benign growths or lesions of the pancreas, and malignant growths or lesions of the pancreas, and metastatic lesions in tissue and organs in the body other than the pancreas. Pancreatic cancer includes all forms of cancer of the pancreas. Pancreatic cancer can include ductal adenocarcinoma, adenosquamous carcinoma, pleomorphic giant cell carcinoma, mucinous adenocarcinoma, osteoclast-like giant cell carcinoma, mucinous cystadenocarcinoma, acinar carcinoma, unclassified large cell carcinoma, small cell carcinoma, pancreatoblastoma, papillary neoplasm, mucinous cystadenoma, papillary cystic neoplasm, and serous cystadenoma. Pancreatic cancer can also include pancreatic neoplasms having histologic and ultrastructual heterogeneity (e.g., mixed cell types).

A ‘cell proliferative disorder of the prostate’ is a cell proliferative disorder involving cells of the prostate. Compounds and compositions of the present application may be used to treat prostate cancer or cell proliferative disorders of the prostate. Cell proliferative disorders of the prostate can include all forms of cell proliferative disorders affecting prostate cells. Cell proliferative disorders of the prostate can include prostate cancer, a precancer or precancerous condition of the prostate, benign growths or lesions of the prostate, and malignant growths or lesions of the prostate, and metastatic lesions in tissue and organs in the body other than the prostate. Cell proliferative disorders of the prostate can include hyperplasia, metaplasia, and dysplasia of the prostate.

A ‘cell proliferative disorder of the skin’ is a cell proliferative disorder involving cells of the skin. Compounds and compositions of the present application may be used to treat skin cancer or cell proliferative disorders of the skin. Cell proliferative disorders of the skin can include all forms of cell proliferative disorders affecting skin cells. Cell proliferative disorders of the skin can include a precancer or precancerous condition of the skin, benign growths or lesions of the skin, melanoma, malignant melanoma and other malignant growths or lesions of the skin, and metastatic lesions in tissue and organs in the body other than the skin. Cell proliferative disorders of the skin can include hyperplasia, metaplasia, and dysplasia of the skin.

A ‘cell proliferative disorder of the ovary’ is a cell proliferative disorder involving cells of the ovary. Compounds and compositions of the present application may be used to treat ovarian cancer or cell proliferative disorders of the ovary. Cell proliferative disorders of the ovary can include all forms of cell proliferative disorders affecting cells of the ovary. Cell proliferative disorders of the ovary can include a precancer or precancerous condition of the ovary, benign growths or lesions of the ovary, ovarian cancer, malignant growths or lesions of the ovary, and metastatic lesions in tissue and organs in the body other than the ovary. Cell proliferative disorders of the skin can include hyperplasia, metaplasia, and dysplasia of cells of the ovary.

A ‘cell proliferative disorder of the breast’ is a cell proliferative disorder involving cells of the breast. Compounds and compositions of the present application may be used to treat breast cancer or cell proliferative disorders of the breast. Cell proliferative disorders of the breast can include all forms of cell proliferative disorders affecting breast cells. Cell proliferative disorders of the breast can include breast cancer, a precancer or precancerous condition of the breast, benign growths or lesions of the breast, and malignant growths or lesions of the breast, and metastatic lesions in tissue and organs in the body other than the breast. Cell proliferative disorders of the breast can include hyperplasia, metaplasia, and dysplasia of the breast.

In one embodiment, the disease or disorder includes, but is not limited to, a disease or disorders caused by or associated with Entamoeba histolytica, Pneumocystis carindi, Trypanosoma cruzi, Trypanosmna brucei, Leishmania mexicana, Clostridium histolyticum, Staphylococcus aureus, foot-and-mouth disease virus, or Crithidia fasciculata, as well as disease or disorder associated with osteoporosis, autoimmunity, schistosomiasis, malaria, tumor metastasis, metachromatic leukodystrophy, muscular dystrophy, or amytrophy.

Additional examples of the diseases or disorders include, but are not limited to, diseases or disorders caused by or associated with veterinary and human pathogenic protozoa, intracellular active parasites of the phylum Apicomplexa or Sarcomastigophora, Trypanosoma, Plasmodia, Leishmania, Babesia and Theileria, Cryptosporidia, Sacrocystida, Amoeba, Coccidia, and Trichomonadia. For example, the diseases or disorders include, but are not limited to, Malaria tropica, caused by, for example, Plasmodium Alciparum; Malaria terdana, caused by Plasmodium vivax or Plasmodium ovale, Malaria quartana, caused by Plasmodium malariae; Toxoplasnosis, caused by Toxoplasma gondii; Coccidiosis, caused for instance by Isospora belli; intestinal Sarcosporidiosis, caused by Sarcocystis suihominis; dysentery caused by Entamoeba histolytica; Cryptosporidiosis, caused by Cryptosporidium parvum; Chagas' disease, caused by Typanosoma cruzi; sleeping sickness, caused by Typanosoma brucei rhodesiense or gambiense, the cutaneous and visceral as well as other forms of Leishmaniosis; diseases or disorders caused by veterinary pathogenic protozoa, such as Theileria parva, the pathogen causing bovine East coast fever, Trypanosoma congolense congolense or Trypanosoma vivax vivax, Trypanosoma brucei brucei, pathogens causing Nagana cattle disease in Africa, Trypanosama brucei evansi causing Surra, Babesia bigemina., the pathogen causing Texas fever in cattle and buffalos, Babesia bovis, the pathogen causing European bovine Babesiosis as well as Babesiosis in dogs, cats and sheep, Sarcocystis ovicanis and ovifelis pathogens causing Sarcocystiosis in sheep, cattle and pigs, Cryptosporidia, pathogens causing Cryptosporidiosis in cattle and birds, Eimeria and Isospora species, pathogens causing Coccidiosis in rabbits, cattle, sheep, goats, pigs and birds, especially in chickens and turkeys. Rickettsia comprise species such as Rickettsia felis, Rickettsia prowazekii, Rickettsia ricketti, Rickettsia typhi, Rickettsia conorii, Rickettsia africae and cause diseases such as typhus, rickettsial pox, Boutonneuse fever, African Tick Bite Fever, Rocky Mountain spotted fever, Australian Tick Typhus, Flinders Island Spotted Fever and Queensland Tick Typhus.

In one embodiment, the disease or disorder is caused by, or associated with, one or more bacteria. Examples of the bacteria include, but are not limited to, the Gram positive organisms (e.g., Staphylococcus aureus, Staphiococcus epidermidis, Enterococcus faecalis and E. faecium, Streptococcus pneumoniae) and the Gram negative organisms (e.g., Pseudomonas aeruginosa, Burkholdia cepacia, Xanthomonas nalophila, Escherichia coli, Enterobacter spp, Klebsiella pneumoniae and Salmonella spp).

In one embodiment, the disease or disorder is caused by, or associated with, one or more fungi. Examples of the fingi include, but are not limited to, Candida albicans, Histoplasma neoformans, Coccidioides immitis, and Penicillium marneffei.

In one embodiment, the disease or disorder is a neurological disease or disorder. In one embodiment, the neurological disease or disorder involves the central nervous system (e.g., brain, brainstem and cerebellum), the peripheral nervous system (e.g., cranial nerves), and/or the autonomic nervous system (e.g., parts of which are located in both central and peripheral nervous system).

Examples of the neurological disorders include, but are not limited to, acquired epileptiform aphasia; acute disseminated encephalomyelitis; adrenoleukodystrophy; age-related macular degeneration; agenesis of the corpus callosurn; agnosia; Aicardi syndrome; Alexander disease; Alpers' disease; alternating hemiplegia; Alzbeimer's disease; Vascular dementia; amyotrophic lateral sclerosis; anencephaly; Angelman syndrome; angiomatosis; anoxia; aphasia; apraxia; arachnoid cysts; arachnoiditis; Anronl-Chiari malformation; arteriovenous malformation; Asperger syndrome; ataxia telegiectasia; attention deficit hyperactivity disorder; autism; autonomic dysfunction; back pain; Batten disease; Behcet's disease; Bell's palsy; benign essential blepharospasm; benign focal; amyotrophy; benign intracranial hypertension; Binswanger's disease; blepharospasm; Bloch Sulzberger syndrome; brachial plexus injury; brain abscess; brain injury; brain tumors (including glioblastoma multiforme); spinal tumor; Brown-Sequard syndrome; Canavan disease; carpal tunnel syndrome; causalgia; central pain syndrome; central pontine myelinolysis; cephalic disorder; cerebral aneurysm; cerebral arteriosclerosis; cerebral atrophy; cerebral gigantism; cerebral palsy; Charcot-Marie-Tooth disease; chemotherapy-induced neuropathy and neuropathic pain; Chiari malformation; chorea; chronic inflammatory demyelinating polyneuropathy; chronic pain; chronic regional pain syndrome; Coffin Lowry syndrome; coma, including persistent vegetative state; congenital facial diplegia; corticobasal degeneration; cranial arteritis; craniosynostosis; Creutzfeldt-Jakob disease; cumulative trauma disorders; Cushing's syndrome; cytomegalic inclusion body disease; cytomegalovirus infection; dancing eyes-dancing feet syndrome; Dandy-Walker syndrome; Dawson disease; De Morsier's syndrome; Dejerine-Klumke palsy; dementia; dermatomyositis; diabetic neuropathy; diffuse sclerosis; dysautonomia; dysgraphia; dyslexia; dystonias; early infantile epileptic encephalopathy; empty sella syndrome; encephalitis; encephaloceles; encephalotrigeminal angiomatosis; epilepsy; Erb's palsy; essential tremor; Fabry's disease; Fahr's syndrome; fainting; familial spastic paralysis; febrile seizures; Fisher syndrome; Friedreich's ataxia; fronto-temporal dementia and other ‘tauopathies’; Gaucher's disease; Gerstmann's syndrome; giant cell arteritis; giant cell inclusion disease; globoid cell leukodystrophy; Guillain-Barre syndrome; HTLV-1-associated myelopathy; Hallervorden-Spatz disease; head injury; headache; hemifacial spasm; hereditary spastic paraplegia; heredopathia atactica polyneuritiformis; herpes zoster oticus; herpes zoster; Hirayama syndrome; HIV-associated dementia and neuropathy (also neurological manifestations of AIDS); holoprosencephaly; Huntington's disease and other polyglutamine repeat diseases; hydranencephaly; hydrocephalus; hypercortisolism; hypoxia; immune-mediated encephalomyelitis; inclusion body myositis; incontinentia pigmenti; infantile phytanic acid storage disease; infantile refsum disease; infantile spasms; inflammatory myopathy; intracranial cyst; intracranial hypertension; Joubert syndrome; Kearns-Sayre syndrome; Kennedy disease Kinsbourne syndrome; Klippel Feil syndrome; Krabbe disease; Kugelberg-Welander disease; kuru; Lafora disease; Lambert-Eaton myasthenic syndrome; Landau-Kleffner syndrome; lateral medullary (Wallenberg) syndrome; leaning disabilities; Leigh's disease; Lennox-Gustaut syndrome; Leseh-Nyhan syndrome; leukodystrophy; Lewy body dementia; Lissencephaly; locked-in syndrome; Lou Giehrig's disease (i.e., motor neuron disease or amyotrophic lateral sclerosis); lumbar disc disease; Lyme disease—neurological sequelae; Machado-Joseph disease; macrencephaly; megalencephaly; Melkersson-Rosenthal syndrome; Menieres disease; meningitis; Menkes disease; metachromatic leukodystrophy; microcephaly; migraine; Miller Fisher syndrome; mini-strokes; mitochondrial myopathies; Mobius syndrome; monomelic amyotrophy; motor neuron disease; Moyamoya disease; mucopolysaccharidoses; multi-infarct dementia; multifocal motor neuropathy; multiple sclerosis and other demyelinating disorders; multiple system atrophy with postural hypotension; p muscular dystrophy; myasthenia gravis; myelinoclastic diffuse sclerosis; nyoclonic encephalopathy of infants; myoclonus; myopathy; myotonia congenital; narcolepsy; neurofibromatosis; neuroleptic malignant syndrome; neurological manifestations of AIDS: neurological sequelae of lupus; neuromyotonia; neuronal ceroid lipofuscinosis; neuronal migration disorders; Niemann-Pick disease; O'Sullivan-McLeod syndrome; occipital neuralgia; occult spinal dysraphism sequence; Ohtahara syndrome; olivopontocerebellar atrophy; opsoclonus myoclonus; optic neuritis; orthostatic hypotension; overuse syndrome; paresthesia; Parkinson's disease; paramyotonia congenital; paraneoplastic diseases; paroxysmal attacks; Parry Romberg syndrome; Pelizaeus-Merzbacher disease; periodic paralyses; peripheral neuropathy; painful neuropathy and neuropathic pain; persistent vegetative state; pervasive developmental disorders; photic sneeze reflex; phytanic acid storage disease; Pick's disease; pinched nerve; pituitary tumors; polymyositis; porencephaly; post-polio syndrome; postherpetic neuralgia; postinfectious encephalomyelitis; postural hypotension; Prader-Willi syndrome; primary lateral sclerosis; prion diseases; progressive hemifacial atrophy; progressive multifocal leukoencephalopathv; progressive sclerosing poliodystrophy; progressive supranuclear palsy; pseudotumor cerebri; Ramsay-Hunt syndrome (types I and II); Rasmussen's encephalitis; reflex sympathetic dystrophy syndrome; Refsum disease; repetitive motion disorders; repetitive stress injuries; restless legs syndrome; retrovirus-associated myelopathy; Rett syndrome; Reye's syndrome; Saint Vitus dance; Sandhoff disease; Schilder's disease; schizencephaly; septo-optic dysplasia; shaken baby syndrome; shingles; Shy-Drager syndrome; Sjögren's syndrome; sleep apnea; Soto's syndrome; spasticity; spina bifida; spinal cord injury; spinal cord tumors; spinal muscular atrophy; Stiff-Person syndrome; stroke; Sturge-Weber syndrome; subacute sclerosing panencephalitis; subcortical arteriosclerotic encephalopathy; Sydenham chorea; syncope; syringomyelia; tardive dyskinesia; Tay-Sachs disease; temporal arteritis; tethered spinal cord syndrome; Thomsen disease; thoracic outlet syndrome; Tic Douloureux; Todd's paralysis; Tourette syndrome; transient ischemic attack; transmissible spongiform encephalopathies; transverse myelitis; traumatic brain injury; tremor; trigeminal neuralgia; tropical spastic paraparesis; tuberous sclerosis; vascular dementia (multi-infarct dementia); vasculitis including temporal arteritis; Von Hippel-Lindau disease; Wallenberg's syndrome; Werdnig-Hoffman disease; West syndrome; whiplash; Williams syndrome; Wildon's disease; and Zellweger syndrome.

Examples of neurodegenerative diseases may also include, without limitation, Adrenoleukodystrophy (ALD), Alexander's disease, Alper's disease, Alzheimer's disease, Amyotrophic lateral sclerosis (Lou Gehrig's Disease), Ataxia telangiectasia, Batten disease (also known as Spielmeyer-Vogt-Sjogren-Batten disease), Bovine spongiform encephalopathy (BSE), Canavan disease, Cockayne syndrome, Corticobasal degeneration, Creutzfeldt-Jakob disease, Familial fatal insomnia, Frontotemporal lobar degeneration, Huntington's disease, HIV-associated dementia, Kennedy's disease, Krabbe's disease, Lewy body dementia, Neuroborreliosis, Machado-Joseph disease (Spinocerebellar ataxia type 3), Multiple System Atrophy, Multiple sclerosis, Narcolepsy, Niemann Pick disease, Parkinson's disease, Pelizaeus-Merzbacher Disease, Pick's disease, Primary lateral sclerosis, Prion diseases, Progressive Supranuclear Palsy, Refsum's disease, Sandhoff disease, Schilder's disease, Subacute combined degeneration of spinal cord secondary to Pernicious Anaemia, Spielmeyer-Vogt-Sjogren-Batten disease (also known as Batten disease), Spinocerebellar ataxia (multiple types with varying characteristics), Spinal muscular atrophy, Steele-Richardson-Olszewski disease, Tabes dorsalis, and Toxic encephalopathy.

In one embodiment, the disease or disorder is an autoimmune disease. Examples of autoimmune diseases include, but are not limited to, rheumatoid arthritis, systemic lupus erythematosus, inflammatory bowel diseases (IBDs) comprising Crohn disease (CD), and ulcerative colitis (UC) which are chronic inflammatory conditions with polygenic susceptibility.

In one embodiment, the disease or disorder is inflammation, arthritis, rheumatoid arthritis, spondyiarthropathies, gouty arthritis, osteoarthritis, juvenile arthritis, and other arthritic conditions, systemic lupus erthematosus (SLE), skin-related conditions, psoriasis, eczema, burns, dermatitis, neuroinflammation, allergy, pain, neuropathic pain, fever, pulmonary disorders, lung inflammation, adult respiratory distress syndrome, pulmonary sarcoisosis, asthma, silicosis, chronic pulmonary inflammatory disease, and chronic obstructive pulmonary disease (COPD), cardiovascular disease, arteriosclerosis, myocardial infarction (including post-myocardial infarction indications), thrombosis, congestive heart failure, cardiac reperfusion injury, as well as complications associated with hypertension and/or heart failure such as vascular organ damage, restenosis, cardiomyopathy, stroke including ischemic and hemorrhagic stroke, reperfusion injury, renal reperfusion injury, ischemia including stroke and brain ischemia, and ischemia resulting from cardiac/coronary bypass, neurodegenerative disorders, liver disease and nephritis, gastrointestinal conditions, inflammatory bowel disease, Crohn's disease, gastritis, irritable bowel syndrome, ulcerative colitis, ulcerative diseases, gastric ulcers, viral and bacterial infections, sepsis, septic shock, gram negative sepsis, malaria, meningitis, HIV infection, opportunistic infections, cachexia secondary to infection or malignancy, cachexia secondary to acquired immune deficiency syndrome (AIDS), AIDS, ARC (AIDS related complex), pneumonia, herpes virus, myalgias due to infection, influenza, autoimmune disease, graft vs. host reaction and allograft rejections, treatment of bone resorption diseases, osteoporosis, multiple sclerosis, cancer, leukemia, lymphoma, colorectal cancer, brain cancer, bone cancer, epithelial call-derived neoplasia (epithelial carcinoma), basal cell carcinoma, adenocarcinoma, gastrointestinal cancer, lip cancer, mouth cancer, esophageal cancer, small bowel cancer, stomach cancer, colon cancer, liver cancer, bladder cancer, pancreas cancer, ovarian cancer, cervical cancer, lung cancer, breast cancer, skin cancer, squamous cell and/or basal cell cancers, prostate cancer, renal cell carcinoma, and other known cancers that affect epithelial cells throughout the body, chronic myelogenous leukemia (CML), acute myeloid leukemia (AML) and acute promyelocytic leukemia (APL), angiogenesis including neoplasia, metastasis, central nervous system disorders, central nervous system disorders having an inflammatory or apoptotic component, Alzheimer's disease, Parkinson's disease, Huntington's disease, amyotrophic lateral sclerosis, spinal cord injury, peripheral neuropathy, or B-Cell Lymphoma.

In one embodiment, the disease or disorder is selected from autoimmune diseases, inflammatory diseases, proliferative and hyper proliferative diseases, immunologically-mediated diseases, bone diseases, metabolic diseases, neurological and neurodegenerative diseases, cardiovascular diseases, hormone related diseases, allergies, asthma, and Alzheimer's disease. In one embodiment, the disease or disorder is selected from a proliferative disorder and an immune disorder.

As modulators of a STING protein, the compounds and compositions of this application are also useful in assessing, studying, or testing biological samples. One aspect of the application relates to modulating the activity of a STING protein in a biological sample, comprising contacting the biological sample with a compound or a composition of the application.

The term ‘biological sample’, as used herein, means an in vitro or an ex vivo sample, including, without limitation, cell cultures or extracts thereof; biopsied material obtained from a mammal or extracts thereof; and blood, saliva, urine, feces, semen, tears, or other body fluids or extracts thereof. Modulation (e.g., inhibition or stimulation) of protein kinase activity in a biological sample is useful for a variety of purposes that are known to one of skill in the art. Examples of such purposes include, but are not limited to, blood transfusion, organ transplantation, and biological specimen storage.

Another aspect of this application relates to the study of a STING protein in biological and pathological phenomena; the study of intracellular signal transduction pathways mediated by STING protein. Examples of such uses include, but are not limited to, biological assays such as enzyme assays and cell-based assays.

The activity of the compounds and compositions of the present application as STING modulators may be assayed in vitro, in vivo, or in a cell line. In vitro assays include assays that determine modulation (e.g., inhibition or stimulation) of binding of a STING ligand to a STING protein through competitive binding assay. Alternate in vitro assays quantitate the ability of the agonist to bind to the protein kinase and may be measured either by radio or fluorescent labelling the agonist prior to binding, isolating the ligand/protein complex and determining the amount of radio/fluorescent label bound. Detailed conditions for assaying a compound utilized in this application as an antagonist of a STING protein are set forth in the Examples below.

In accordance with the foregoing, the present application provides a method for preventing or treating any of the diseases or disorders described above in a subject in need of such treatment, comprising administering to the subject a therapeutically effective amount of a compound of the application or an enantiomer, diastereomer, stereoisomer, or pharmaceutically acceptable salt thereof, or a pharmaceutical composition of the application. For any of the above uses, the required dosage will vary depending on the mode of administration, the particular condition to be treated and the effect desired.

Compounds and compositions of the application can be administered in therapeutically effective amounts in a combinational therapy with one or more therapeutic agents (pharmaceutical combinations) or modalities, e.g., anti-proliferative, anti-cancer, immunomodulatory, or anti-inflammatory agent, and/or non-drug therapies, etc. For example, synergistic effects can occur with anti-proliferative, anti-cancer, immunomodulatory, or anti-inflammatory substances. Where the compounds of the application are administered in conjunction with other therapies, dosages of the co-administered compounds will of course vary depending on the type of co-drug employed, on the specific drug employed, on the condition being treated and so forth.

Combination therapy may include the administration of the subject compounds in further combination with one or more other biologically active ingredients (such as, but not limited to, a second STING modulator (inhibitor or stimulator), a modulator (inhibitor or stimulator) of the cGAS-CDN-STING axis, or a modulator (inhibitor or stimulator) involved in the intracellular dsDNA mediated type-˜ interferon activation. U.S. patent application Ser. No. 16/717,325 entitled Modulating Immune Responses inventor Glen N. Barber, filed Dec. 17, 2019 is herein incorporated by reference in its entirety and for all purposes. Other biologically active ingredients may also include anti-proliferative agents, anti-cancer agents (e.g., chemotherapeutic agents), immunomodulatory agents, antibodies, etc. For instance, the compounds of the application can be used in combination with other pharmaceutically active compounds, preferably compounds that are able to enhance the agonist effect of the compounds of the application. The compounds of the application can be administered simultaneously (as a single preparation or separate preparation) or sequentially to the other drug therapy or treatment modality. In general, a combination therapy envisions administration of two or more drugs during a single cycle or course of therapy.

In one embodiment, the chemotherapeutic agent is an alkylating agent; an antibiotic; an anti-metabolite; a detoxifying agent; an interferon; a polyclonal or monoclonal antibody; an EGFR inhibitor; a HER2 inhibitor; a histone deacetylase inhibitor; a hormone; a mitotic inhibitor; an MTOR inhibitor; a multi-kinase inhibitor; a serine/threonine kinase inhibitor; a tyrosine kinase inhibitors; a VEGF/VEGFR inhibitor; a taxane or taxane derivative, an aromatase inhibitor, an anthracycline, a microtubule targeting drug, a topoisomerase poison drug, an inhibitor of a molecular target or enzyme (e.g., a kinase inhibitor), a cytidine analog drug, or any chemotherapeutic, anti-neoplastic or anti-proliferative agent listed in www.cancer.org/docroot/cdg/cdg_0.asp, last visited Apr. 27, 2020.

Exemplary alkylating agents include, but are not limited to, cyclophosphamide (Cytoxan; Neosar); chlorambucil (Leukeran); melphalan (Alkeran); carmustine (BiCNU); busulfan (Busulfex); lomustine (CeeNU); dacarbazine (DTIC-Dome); oxaliplatin (Eloxatin); carmustine (Gliadel); ifosfamide (Ifex); mechlorethamine (Mustargen); busulfan (Myleran); carboplatin (Paraplatin); cisplatin (CDDP; Platinol); temozolomide (Temodar); thiotepa (Thioplex); bendamustine (Treanda); or streptozocin (Zanosar).

Exemplary antibiotics include, but are not limited to, doxorubicin (Adriamycin); doxorubicin liposomal (Doxil); mitoxantrone (Novantrone); bleomycin (Blenoxane); daunorubicin (Cerubidine); daunorubicin liposomal (DaunoXome); dactinomycin (Cosmegen); epirubicin (Ellence); idarubicin (Idamycin); plicamycin (Mithracin); mitomycin (Mutamycin); pentostatin (Nipent); or valrubicin (Valstar). Exemplary anti-metabolites include, but are not limited to, fluorouracil (Adrucil); capecitabine (Xeloda); hydroxyurea (Hydrea); mercaptopurine (Purinethol); pemetrexed (Alimta); fludarabine (Fludara); nelarabine (Arranon); cladribine (Cladribine Novaplus); clofarabine (Clolar); cytarabine (Cytosar-U); decitabine (Dacogen); cytarabine liposomal (DepoCyt); hydroxyurea (Droxia); pralatrexate (Folotyn); floxuridine (FUDR); gemcitabine (Gemzar); cladribine (Leustatin); fludarabine (Oforta); methotrexate (MTX; Rheumatrex); methotrexate (Trexall); thioguanine (Tabloid); TS-1 or cytarabine (Tarabine PFS). Exemplary detoxifying agents include, but are not limited to, amifostine (Ethyol) or mesna (Mesnex). Exemplary interferons include, but are not limited to, interferon alfa-2b (Intron A) or interferon alfa-2a (Roferon-A). Exemplary polyclonal or monoclonal antibodies include, but are not limited to, trastuzumab (Herceptin); ofatumumab (Arzerra); bevacizumab (Avastin); rituximab (Rituxan); cetuximab (Erbitux); panitumumab (Vectibix); tositumomab/iodine³′ tositumomab (Bexxar); alemtuzumab (Campath); ibritumomab (Zevalin; In-111; Y-90 Zevalin); gemtuzumab (Mylotarg); eculizumab (Soliris) ordenosumab.

Exemplary EGFR inhibitors include, but are not limited to, gefitinib (Iressa); lapatinib (Tykerb); cetuximab (Erbitux); erlotinib (Tarceva); panitumumab (Vectibix); PKI-166; canertinib (CI-1033); matuzumab (Emd7200) or EKB-569. Exemplary HER2 inhibitors include, but are not limited to, trastuzumab (Herceptin); lapatinib (Tykerb) or AC-480. Exemplary histone Deacetylase Inhibitors include, but are not limited to, vorinostat (Zolinza). Exemplary hormones include, but are not limited to, tamoxifen (Soltamox; Nolvadex); raloxifene (Evista); megestrol (Megace); leuprolide (Lupron; Lupron Depot; Eligard; Viadur); fulvestrant (Faslodex); letrozole (Femara); triptorelin (Trelstar LA; Trelstar Depot); exemestane (Aromasin); goserelin (Zoladex); bicalutamide (Casodex); anastrozole (Arimidex); fluoxymesterone (Androxy; Halotestin); medroxyprogesterone (Provera; Depo-Provera); estramustine (Emcyt); flutamide (Eulexin); toremifene (Fareston); degarelix (Firmagon); nilutamide (Nilandron); abarelix (Plenaxis); or testolactone (Teslac).

Exemplary mitotic inhibitors include, but are not limited to, paclitaxel (Taxol; Onxol; Abraxane); docetaxel (Taxotere); vincristine (Oncovin; Vincasar PFS); vinblastine (Velban); etoposide (Toposar; Etopophos; VePesid); teniposide (Vumon); ixabepilone (Ixempra); nocodazole; epothilone; vinorelbine (Navelbine); camptothecin (CPT); irinotecan (Camptosar); topotecan (Hycamtin); amsacrine or lamellarin D (LAM-D). Exemplary MTOR inhibitors include, but are not limited to, everolimus (Afinitor) or temsirolimus (Torisel); rapamune, ridaforolimus; or AP23573. Exemplary multi-kinase inhibitors include, but are not limited to, sorafenib (Nexavar); sunitinib (Sutent); BIBW 2992; E7080; Zd6474; PKC-412; motesanib; or AP24534. Exemplary serine/threonine kinase inhibitors include, but are not limited to, ruboxistaurin; eril/easudil hydrochloride; flavopiridol; seliciclib (CYC202; Roscovitrine); SNS-032 (BMS-387032); Pkc412; bryostatin; KAI-9803; SF1126; VX-680; Azd1152; Arry-142886 (AZD-6244); SCIO-469; GW681323; CC-401; CEP-1347 or PD 332991. Exemplary tyrosine kinase inhibitors include, but are not limited to, erlotinib (Tarceva); gefitinib (Iressa); imatinib (Gleevec); sorafenib (Nexavar); sunitinib (Sutent); trastuzumab (Herceptin); bevacizumab (Avastin); rituximab (Rituxan); lapatinib (Tykerb); cetuximab (Erbitux); panitumumab (Vectibix); everolimus (Afinitor); alemtuzumab (Campath); gemtuzumab (Mylotarg); temsirolimus (Torisel); pazopanib (Votrient); dasatinib (Sprycel); nilotinib (Tasigna); vatalanib (Ptk787; ZK222584); CEP-701; SU5614; MLN518; XL999; VX-322; Azd0530; BMS-354825; SKI-606 CP-690; AG-490; WHI-P154; WHI-P131; AC-220; or AMG888.

Exemplary VEGF/VEGFR inhibitors include, but are not limited to, bevacizumab (Avastin); sorafenib (Nexavar); sunitinib (Sutent); ranibizumab; pegaptanib; or vandetinib. Exemplary microtubule targeting drugs include, but are not limited to, paclitaxel, docetaxel, vincristin, vinblastin, nocodazole, epothilones and navelbine. Exemplary topoisomerase poison drugs include, but are not limited to, teniposide, etoposide, adriamycin, camptothecin, daunorubicin, dactinomycin, mitoxantrone, amsacrine, epirubicin and idarubicin. Exemplary taxanes or taxane derivatives include, but are not limited to, paclitaxel and docetaxol.

Exemplary general chemotherapeutic, anti-neoplastic, anti-proliferative agents include, but are not limited to, altretamine (Hexalen); isotretinoin (Accutane; Amnesteem; Claravis; Sotret); tretinoin (Vesanoid); azacitidine (Vidaza); bortezomib (Velcade) asparaginase (Elspar); levamisole (Ergamisol); mitotane (Lysodren); procarbazine (Matulane); pegaspargase (Oncaspar); denileukin diftitox (Ontak); porfimer (Photofrin); aldesleukin (Proleukin); lenalidomide (Revlimid); bexarotene (Targretin); thalidomide (Thalomid); temsirolimus (Torisel); arsenic trioxide (Trisenox); verteporfin (Visudyne); mimosine (Leucenol); (1M tegafur—0.4 M 5-chloro-2,4-dihydroxypyrimidine—1 M potassium oxonate) or lovastatin.

Exemplary kinase inhibitors include, but are not limited to, Bevacizumab (targets VEGF), BIBW 2992 (targets EGFR and Erb2), Cetuximab/Erbitux (targets Erb1), Imatinib/Gleevic (targets Bcr-Abl), Trastuzumab (targets Erb2), Gefitinib/Iressa (targets EGFR), Ranibizumab (targets VEGF), Pegaptanib (targets VEGF), Erlotinib/Tarceva (targets Erb1), Nilotinib (targets Bcr-Abl), Lapatinib (targets Erb1 and Erb2/Her2), GW-572016/lapatinib ditosylate (targets HER2/Erb2), Panitumumab/Vectibix (targets EGFR), Vandetinib (targets RET/VEGFR), E7080 (multiple targets including RET and VEGFR), Herceptin (targets HER2/Erb2), PKI-166 (targets EGFR), Canertinib/CI-1033 (targets EGFR), Sunitinib/SU-11464/Sutent (targets EGFR and FLT3), Matuzumab/Emd7200 (targets EGFR), EKB-569 (targets EGFR), Zd6474 (targets EGFR and VEGFR), PKC-412 (targets VEGR and FLT3), Vatalanib/Ptk787/ZK222584 (targets VEGR), CEP-701 (targets FLT3), SU5614 (targets FLT3), MLN518 (targets FLT3), XL999 (targets FLT3), VX-322 (targets FLT3), Azd0530 (targets SRC), BMS-354825 (targets SRC), SKI-606 (targets SRC), CP-690 (targets JAK), AG-490 (targets JAK), WHI-P154 (targets JAK), WHI-P131 (targets JAK), sorafenib/Nexavar (targets RAF kinase, VEGFR-1, VEGFR-2, VEGFR-3, PDGFR-β, KIT, FLT-3, and RET), Dasatinib/Sprycel (BCR/ABL and Src), AC-220 (targets Flt3), AC-480 (targets all HER proteins, ‘panHER’), Motesanib diphosphate (targets VEGF1-3, PDGFR, and c-kit), Denosumab (targets RANKL, inhibits SRC), AMG888 (targets HER3), and AP24534 (multiple targets including Flt3).

In one embodiment, the compounds may be administered in combination with one or more separate pharmaceutical agents, e.g., a chemotherapeutic agent, an immunotherapeutic agent, or an adjunctive therapeutic agent.

As used herein, ‘combination therapy’ or ‘co-therapy’ includes the administration of a compound of the present application, or a pharmaceutically acceptable salt or ester thereof, and at least a second agent as part of a specific treatment regimen intended to provide the beneficial effect from the co-action of these therapeutic agents. The beneficial effect of the combination includes, but is not limited to, pharmacokinetic or pharmacodynamic co-action resulting from the combination of therapeutic agents. Administration of these therapeutic agents in combination typically is carried out over a defined time period (usually minutes, hours, days or weeks depending upon the combination selected). ‘Combination therapy’ may be, but generally is not, intended to encompass the administration of two or more of these therapeutic agents as part of separate monotherapy regimens that incidentally and arbitrarily result in the combinations of the present application.

‘Combination therapy’ is intended to embrace administration of these therapeutic agents in a sequential manner, wherein each therapeutic agent is administered at a different time, as well as administration of these therapeutic agents, or at least two of the therapeutic agents, in a substantially simultaneous manner. Substantially simultaneous administration can be accomplished, for example, by administering to the subject a single capsule having a fixed ratio of each therapeutic agent or in multiple, single capsules for each of the therapeutic agents. Sequential or substantially simultaneous administration of each therapeutic agent can be effected by any appropriate route including, but not limited to, oral routes, i.v. routes, intramuscular routes, and direct absorption through mucous membrane tissues. The therapeutic agents can be administered by the same route or by different routes. For example, a first therapeutic agent of the combination selected may be administered by i.v. injection while the other therapeutic agents of the combination may be administered orally. Alternatively, for example, all therapeutic agents may be administered orally or all therapeutic agents may be administered by i.v. injection. The sequence in which the therapeutic agents are administered is not narrowly critical.

‘Combination therapy’ also embraces the administration of the therapeutic agents as described above in further combination with other biologically active ingredients and non-drug therapies (e.g., surgery or radiation treatment). Where the combination therapy further comprises a non-drug treatment, the non-drug treatment may be conducted at any suitable time so long as a beneficial effect from the co-action of the combination of the therapeutic agents and non-drug treatment is achieved. For example, in appropriate cases, the beneficial effect is still achieved when the non-drug treatment is temporally removed from the administration of the therapeutic agents, perhaps by days or even weeks.

The compounds of this application may be modified by appending various functionalities via any synthetic means delineated herein to enhance selective biological properties. Such modifications are known in the art and include those which increase biological penetration into a given biological system (e.g., blood, lymphatic system, central nervous system), increase oral availability, increase solubility to allow administration by injection, alter metabolism and alter rate of excretion.

### Check Point Inhibitors

The anti-PD-L1 (IgG BE0091, BE0101 or J43 BE0033-2, BioXcell, NH) and anti-PD1 (CD279, BioXcell, NH) were used in the B16 melanoma model.

### Biological Assays

Biological activities of the compounds of the present application can be measured by various biochemical or cellular assays known to one of ordinary skill in the art. Non-limiting examples of biochemical and cellular assays are listed in the Examples vide infra.

### Pharmaceutical Compositions

In another aspect, a pharmaceutical composition is provided. The pharmaceutical composition comprises a therapeutically effective amount of a compound of the application, or a pharmaceutically acceptable salt or ester thereof, and a pharmaceutically acceptable carrier.

Compounds of the application may be administered as pharmaceutical compositions by any conventional route, in particular internally, e.g., orally, e.g., in the form of tablets or capsules, or parenterally, e.g., in the form of injectable solutions or suspensions, or topically, e.g., in the form of lotions, gels, ointments or creams, or in a nasal or suppository form.

### Example 0

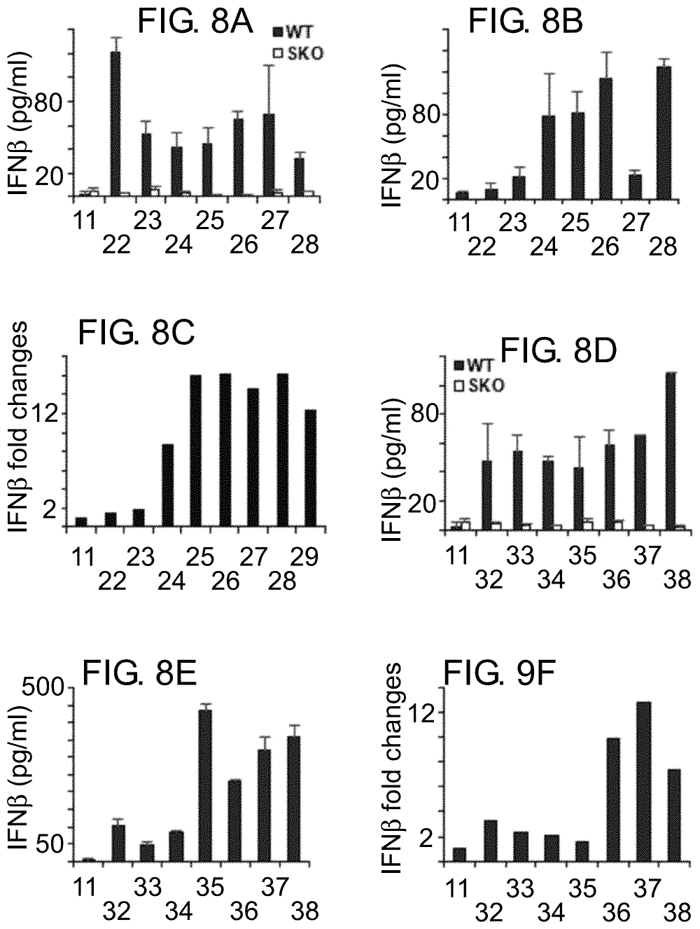

In an embodiment of the present invention, a variety of dsDNA and ssDNA species, that varied in their GC or AT content were evaluated to determine which STAVs were better at stimulating STING signaling following transfection of normal human and mouse cells including APCs. In an embodiment of the present invention, a number of STAVs that were synthetically generated and contained exonuclease resistant phosphorothioates at the ends (ES). STAVs that were greater than 70 bp were found to be effective in stimulating STING-based cytokine production (FIG. 8). FIGS. 8A-F show results for various STING ligands with different sequences and length. FIG. 8A and FIG. 8B show IFN3 ELISA assay in mouse embryonic fibroblasts (MEFs), hTERT transfected with different lengths of AT rich-STING ligands (A:T30ES (22, A:T50ES (23, A:T60ES (24, A:T70ES (25, A:T80ES (26, A:T90ES (27), and A:T100ES (28). FIG. 8C shows qRT-PCR analysis of IFN31 in human macrophages transfected with different length of AT rich-STING ligands. FIG. 8D and FIG. 8E show IFN3 ELISA assays in MEFs, hTERT transfected with different lengths of GC rich-STING ligands (GC30ES 32, GC50ES 33, GC60ES 34, GC70ES 35, GC80ES 36, GC90ES 37, and GC100ES 38). FIG. 8F shows qRT-PCR analysis of IFN31 in human macrophages transfected with different length of GC rich-STING ligands (GC30ES 32, GC50ES 33, GC60ES 34, GC70ES 35, GC80ES 36, GC90ES 37, and GC100ES 38).

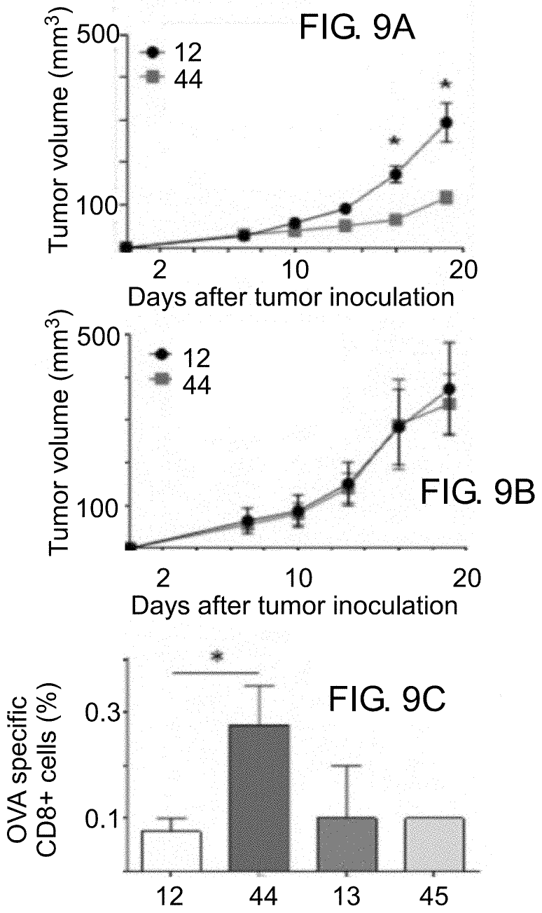

In an embodiment of the present invention, a first STAVs for primary inoculation (AT rich) can be used and a second STAVs for boosting purposes (GC) rich can be used to avoid autoimmune targeting of the STAV itself. In an embodiment of the present invention, AT rich STAVs (80 bp) are used. The STAVs were inoculated into tumors (B16-OVA) grown on the flanks of C57/BL6 mice. Unexpectedly, FIGS. 2A-C show significant anti-tumor activity of STAVs in B16 OVA melanoma bearing mice with intact STING signaling resulting in regression of tumors. The mice were subcutaneously injected with B16-OVA cells on the flank. 10 μg of STAVs were injected i.t. in B16 OVA melanomas every 3 days. The treatment show tumor volumes from WT (n=7/group) from WT (n=4/group) mice injected with STAV1-STAV5 44 or PBS as control 12 (see FIG. 9A) and STING knock out (SKO) mice (n=7/group) SKO (n=4/group) mice injected with STAV1-STAV5 45 or PBS as control 13 (see FIG. 9B) measured on the indicated days. FIG. 9C shows the frequency of OVA specific CD8+ T cells in the spleen from WT (n=4/group) mice injected with STAV1-STAV5 44 or PBS as control 12. FIG. 9C also shows the frequency of OVA specific CD8+ T cells in the spleen from SKO (n=4/group) mice injected with STAV1-STAV5 45 or PBS as control 13. Unexpectedly, the STAV reduces the tumor volume by more than half in the wild type mice with the intact STING gene.

To complement this approach, tumor cells (B16 melanoma) were loaded with polyA90ES-FAM and polyT90ES-FAM (5′ fluorescently labelled STAVs referred to as STAVs-FAM (see SEQ ID NO:35, SEQ ID NO:36). The STAV-FAMs were used to visualize the STAVs location in the cells. Greater than 90% of the B16 cells took up the STAVs following transfection (data not shown).

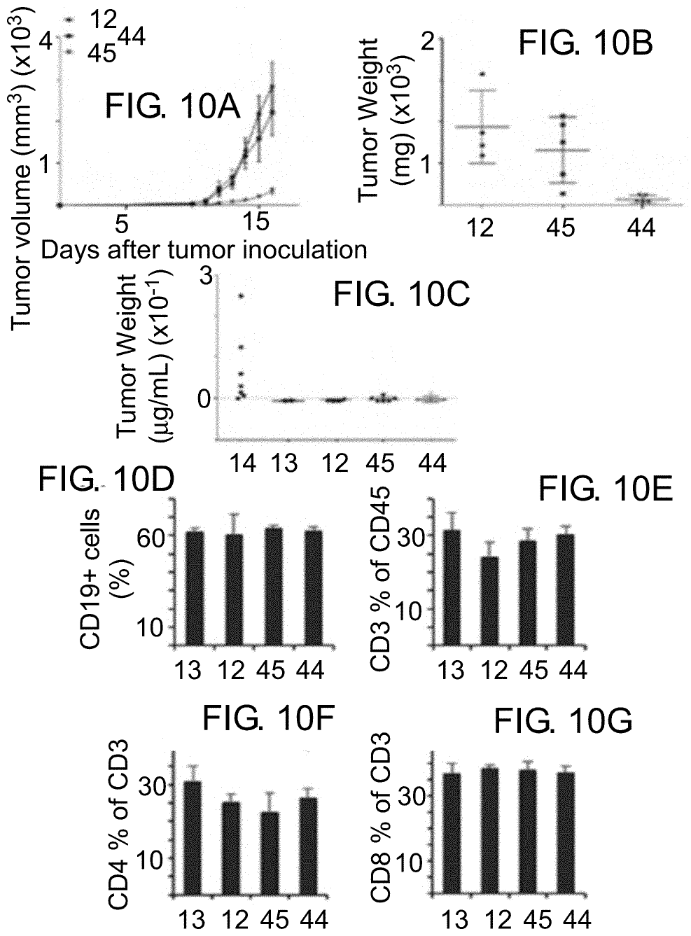

C57/BL6 mice were inoculated with C1498 (murine AML) cells, where the C1498 cells were transfected with STAVs (3 μg/ml) for 3 hours and irradiated by UV (120 mJ/cm for 1 minute) and incubated for 24 hours, followed by sequential intraperitoneal injections of the UV irradiated AML cells loaded with three (3) distinct STAVs sequences STAV1 on Day 2, STAV2 on Day 5, and STAV3 on Day 10 (FIG. 10). Unexpectedly, the STAVs-based dead cell therapy abolished AML tumor growth as evidenced by marked reduction in tumor volume and tumor weight as compared to control (PBS) and untreated groups (see FIGS. 10A and 10B). Also unexpectedly, no evidence of antibodies directed against STAV1, STAV2 or STAV3 or double stranded DNA were detected from mouse sera (see FIG. 10C), and no negative effect was observed on CD19+, CD3, CD4, CD8 and CD45 normal immune cell populations (see FIGS. 10D-10G). STAVs-based dead cell therapy was similarly effective in blocking EL4 (murine T-cell ALL model) tumor growth in immunocompetent mice (data not shown).

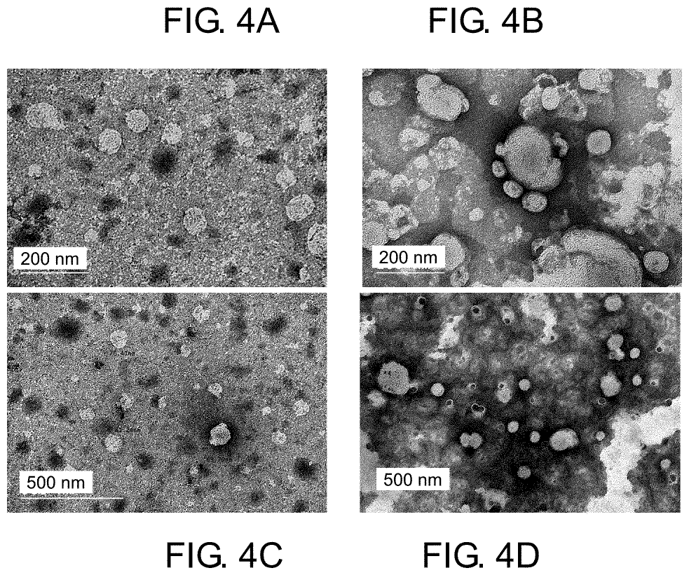

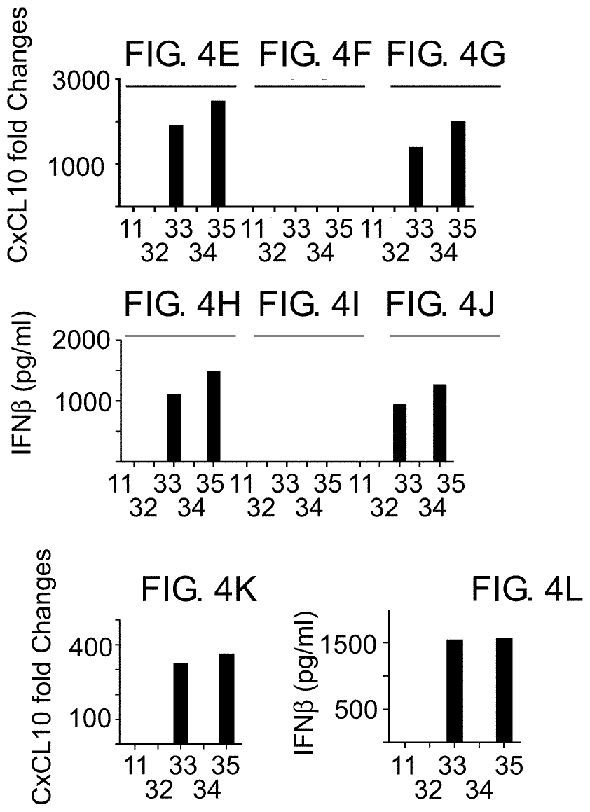

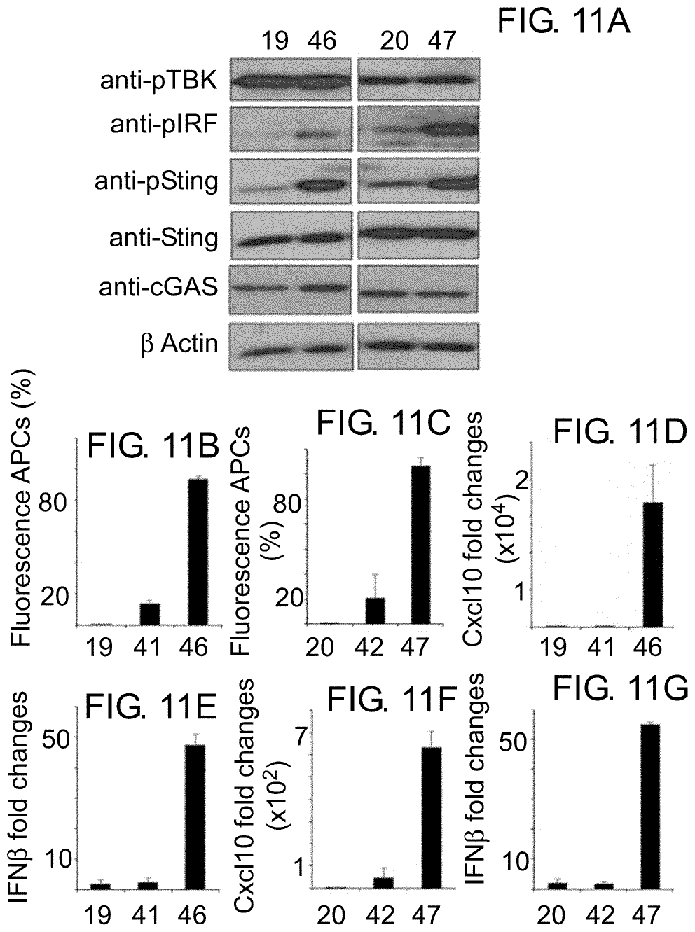

The integrity of the STING-IRF3 signaling pathway in AML leukemia cells, transfected with interferon stimulatory DNA (ISD) 46 compared with Mock 19, and ATLL (ATLL-84c or JAE) leukemia cells, transfected with ISD 47 compared with Mock 20, was confirmed to result in phosphorylation (activation) of STING and IRF-3, which is known to be activated by STING (see FIG. 11A for immunoblots revealing phosphorylation of STING (pSTING) and IRF-3 (pIRF3) 4 hours after transfecting with ISD relative to unphosphorylated forms of STING, IRF3, and pTBK (cGAS, and si-Actin, controls). The presence of fluorescein (FAM) labelled STAVs was confirmed in macrophages after phagocytosis of UV irradiated AML and ATLL cells transfected with STAVs (FIG. 11B and FIG. 11C). UV irradiated AML cells loaded with FAM labelled STAVs 46 resulted in robust production of CXCL10 in human macrophages (FIG. 11D, Mock 19 and UV irradiated only 41 and of IFNB1 also in human macrophages (FIG. 11E, Mock 19 and UV irradiated only 41. Finally, UV irradiated ATLL cells loaded with FAM labelled STAVs 47 resulted in robust production of CXCL10 in human macrophages (FIG. 11F, Mock 20 and UV irradiated only 42 and IFNB1 also in human macrophages (FIG. 4G, Mock 20 and UV irradiated only 42.

An anti-tumor therapy against re-infusible tumors, such as leukemia by treating patient's tumors with STAVs, irradiating, and re-infusing. The tumor cells can be engulfed by APC's and the tumor specific proteins presented on MHC can prime anti-tumor T cells. EL4 grows in the flanks of C57/BL6 mice or can metastasize to the lungs. EL4-HBZ cells loaded with STAVs potently activate APCs in a STING-dependent manner.

Using the dead cell immunization protocol, EL4 or EL4-cGAS cells were transfected with STAV1 (3 μg/mL) for 3 hours and UV irradiated (120 mJ/cm for 1 minute) (after 24 hours, C57/BL6 (wild) type mice were injected i.p. with the irradiated EL4 or EL4-cGAS cells with/without STAVs, after the primary injection, mice were boosted with the irradiated EL4 or EL4-cGAS cells with/without STAVs at Day 16.

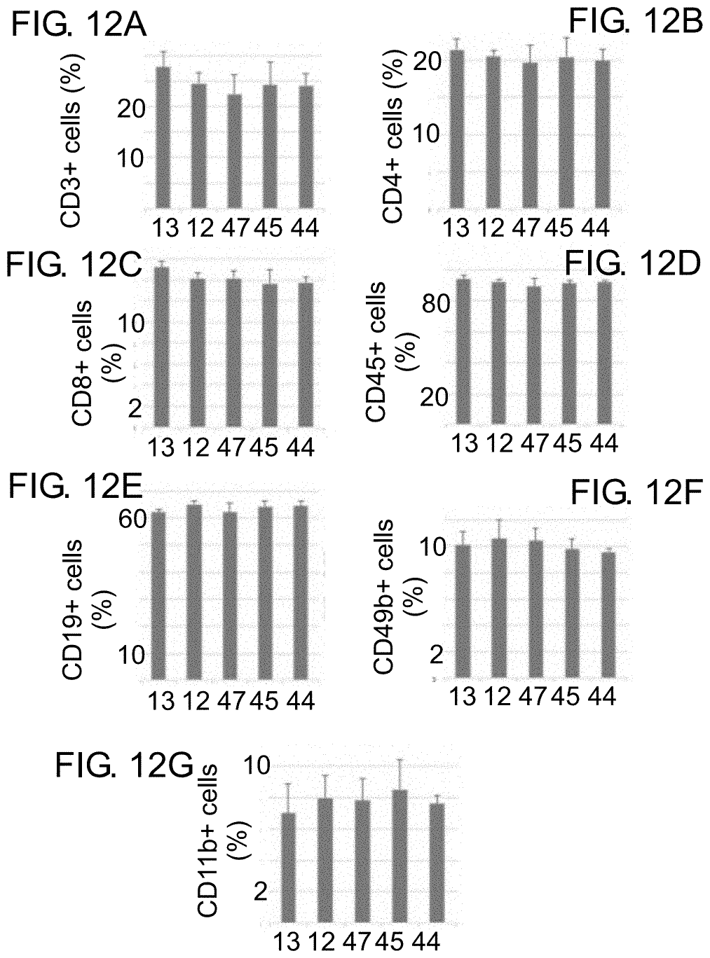

FIG. 12A shows flow cytometry analysis of splenocytes isolated and stained with the fluorescently labeled anti-CD3-FITC antibodies from C57/BL56 mice sacrificed on day 25 (9 days after a first boost), where the splenocytes were isolated from the mice i.p. injected with PBS 13, EL4 cells 12, EL4 cells transfected with STAV1 47, EL4-cGAS cells 45, or EL4-cGAS cells transfected with STAV1 45 (the mice were i.p. injected 24 hours after the cells were transfected with STAV1 (3 μg/mL) for 3 hours and UV irradiated (120 mJ/cm for 1 minute) and boosted with the STAV1 after 16 days.

FIG. 12B shows flow cytometry analysis of splenocytes isolated and stained with the fluorescently labeled anti-CD4-PE antibodies from C57/BL56 mice sacrificed on day 25 (9 days after a first boost), where the splenocytes were isolated from the mice i.p. injected with PBS 13, EL4 cells 12, EL4 cells transfected with STAV1 47, EL4-cGAS cells 45, or EL4-cGAS cells transfected with STAV1 45 (the mice were i.p. injected 24 hours after the cells were transfected with STAV1 (3 μg/mL) for 3 hours and UV irradiated (120 mJ/cm for 1 minute) and boosted with the STAV1 after 16 days.

FIG. 12C shows flow cytometry analysis of splenocytes isolated and stained with the fluorescently labeled anti-CD8a-PercP antibodies from C57/BL56 mice sacrificed on day 25 (9 days after a first boost), where the splenocytes were isolated from the mice i.p. injected with PBS 13, EL4 cells 12, EL4 cells transfected with STAV1 47, EL4-cGAS cells 45, or EL4-cGAS cells transfected with STAV1 45 (the mice were i.p. injected 24 hours after the cells were transfected with STAV1 (3 μg/mL) for 3 hours and UV irradiated (120 mJ/cm for 1 minute) and boosted with the STAV1 after 16 days.

FIG. 12D shows flow cytometry analysis of splenocytes isolated and stained with the fluorescently labeled anti-CD45-Pacific Blue antibodies from C57/BL56 mice sacrificed on day 25 (9 days after a first boost), where the splenocytes were isolated from the mice i.p. injected with PBS 13, EL4 cells 12, EL4 cells transfected with STAV1 47, EL4-cGAS cells 45, or EL4-cGAS cells transfected with STAV1 45 (the mice were i.p. injected 24 hours after the cells were transfected with STAV1 (3 μg/mL) for 3 hours and UV irradiated (120 mJ/cm for 1 minute) and boosted with the STAV1 after 16 days.

FIG. 12E shows flow cytometry analysis of splenocytes isolated and stained with the fluorescently labeled anti-CD19-Alexa Fluor 700 antibodies from C57/BL56 mice sacrificed on day 25 (9 days after a first boost), where the splenocytes were isolated from the mice i.p. injected with PBS 13, EL4 cells 12, EL4 cells transfected with STAV1 47, EL4-cGAS cells 45, or EL4-cGAS cells transfected with STAV1 45 (the mice were i.p. injected 24 hours after the cells were transfected with STAV1 (3 μg/mL) for 3 hours and UV irradiated (120 mJ/cm for 1 minute) and boosted with the STAV1 after 16 days.

FIG. 12F shows flow cytometry analysis of splenocytes isolated and stained with the fluorescently labeled anti-CD49b-PE/Cy7 antibodies from C57/BL56 mice sacrificed on day 25 (9 days after a first boost), where the splenocytes were isolated from the mice i.p. injected with PBS 13, EL4 cells 12, EL4 cells transfected with STAV1 47, EL4-cGAS cells 45, or EL4-cGAS cells transfected with STAV1 45 (the mice were i.p. injected 24 hours after the cells were transfected with STAV1 (3 μg/mL) for 3 hours and UV irradiated (120 mJ/cm for 1 minute) and boosted with the STAV1 after 16 days.

FIG. 12G shows flow cytometry analysis of splenocytes isolated and stained with the fluorescently labeled anti-CD11b-FITC antibodies from C57/BL56 mice sacrificed on day 25 (9 days after a first boost), where the splenocytes were isolated from the mice i.p. injected with PBS 13, EL4 cells 12, EL4 cells transfected with STAV1 47, EL4-cGAS cells 45, or EL4-cGAS cells transfected with STAV1 45 (the mice were i.p. injected 24 hours after the cells were transfected with STAV1 (3 μg/mL) for 3 hours and UV irradiated (120 mJ/cm for 1 minute) and boosted with the STAV1 after 16 days.

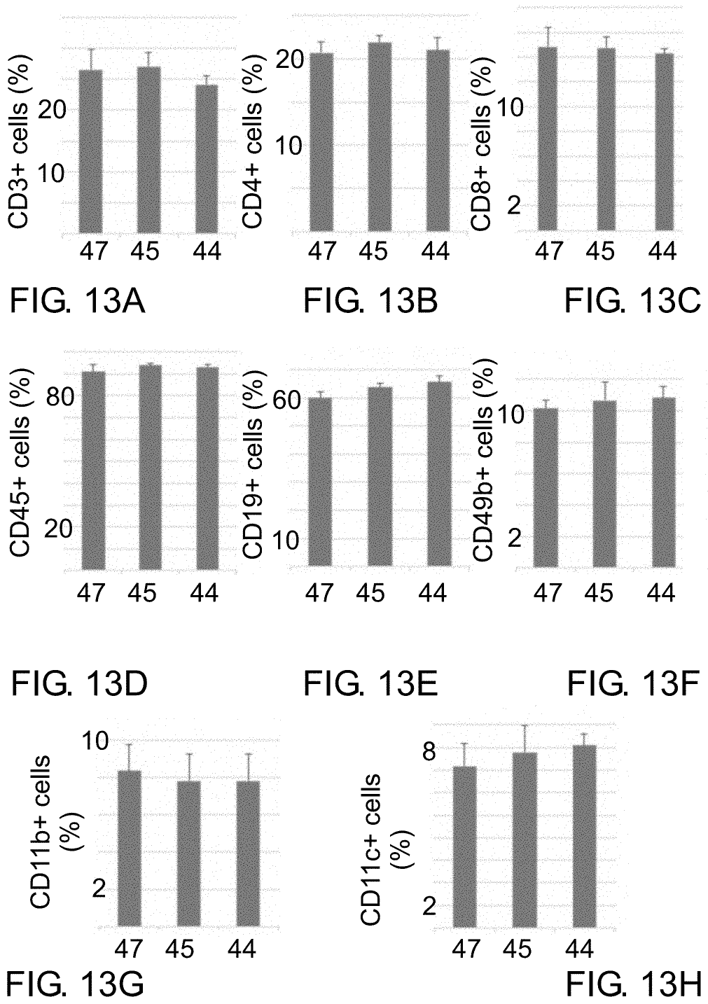

FIG. 13A shows flow cytometry analysis of splenocytes isolated and stained with the fluorescently labeled anti-CD3-FITC antibodies from C57/BL56 mice sacrificed on day 12, where the splenocytes were isolated from the mice i.p. injected with C1498 cells (24 hours after the cells were transfected with STAV1 (3 μg/mL) for 3 hours and UV irradiated (120 mJ/cm for 1 minute) 44, or the UV irradiated cells (only) 45, and the PBS control 47 and boosted with the STAV1 after 6 days.

FIG. 13B shows flow cytometry analysis of splenocytes isolated and stained with the fluorescently labeled anti-CD4-PE antibodies from C57/BL56 mice sacrificed on day 12, where the splenocytes were isolated from the mice i.p. injected with C1498 cells (24 hours after the cells were transfected with STAV1 (3 μg/mL) for 3 hours and UV irradiated (120 mJ/cm for 1 minute) 44, or the UV irradiated cells (only) 45, and the PBS control 47 and boosted with the STAV1 after 6 days.

FIG. 13C shows flow cytometry analysis of splenocytes isolated and stained with the fluorescently labeled anti-CD8-PercP antibodies from C57/BL56 mice sacrificed on day 12, where the splenocytes were isolated from the mice i.p. injected with C1498 cells (24 hours after the cells were transfected with STAV1 (3 μg/mL) for 3 hours and UV irradiated (120 mJ/cm for 1 minute) 44, or the UV irradiated cells (only) 45, and the PBS control 47 and boosted with the STAV1 after 6 days.

FIG. 13D shows flow cytometry analysis of splenocytes isolated and stained with the fluorescently labeled anti-CD45-Pacific Blue antibodies from C57/BL56 mice sacrificed on day 12, where the splenocytes were isolated from the mice i.p. injected with C1498 cells (24 hours after the cells were transfected with STAV1 (3 μg/mL) for 3 hours and UV irradiated (120 mJ/cm for 1 minute) 44, or the UV irradiated cells (only) 45, and the PBS control 47 and boosted with the STAV1 after 6 days.

FIG. 13E shows flow cytometry analysis of splenocytes isolated and stained with the fluorescently labeled anti-CD19-Alexa Fluor 700 antibodies from C57/BL56 mice sacrificed on day 12, where the splenocytes were isolated from the mice i.p. injected with C1498 cells (24 hours after the cells were transfected with STAV1 (3 μg/mL) for 3 hours and UV irradiated (120 mJ/cm for 1 minute) 44, or the UV irradiated cells (only) 45, and the PBS control 47 and boosted with the STAV1 after 6 days.

FIG. 13F shows flow cytometry analysis of splenocytes isolated and stained with the fluorescently labeled anti-CD49b-PE/Cy7 antibodies from C57/BL56 mice sacrificed on day 12, where the splenocytes were isolated from the mice i.p. injected with C1498 cells (24 hours after the cells were transfected with STAV1 (3 μg/mL) for 3 hours and UV irradiated (120 mJ/cm for 1 minute) 44, or the UV irradiated cells (only) 45, and the PBS control 47 and boosted with the STAV1 after 6 days.

FIG. 13G shows flow cytometry analysis of splenocytes isolated and stained with the fluorescently labeled anti-CD11b-FITC antibodies from C57/BL56 mice sacrificed on day 12, where the splenocytes were isolated from the mice i.p. injected with C1498 cells (24 hours after the cells were transfected with STAV1 (3 μg/mL) for 3 hours and UV irradiated (120 mJ/cm for 1 minute) 44, or the UV irradiated cells (only) 45, and the PBS control 47 and boosted with the STAV1 after 6 days.

FIG. 13H shows flow cytometry analysis of splenocytes isolated and stained with the fluorescently labeled anti-CD11c-FITC antibodies from C57/BL56 mice sacrificed on day 12, where the splenocytes were isolated from the mice i.p. injected with C1498 cells (24 hours after the cells were transfected with STAV1 (3 μg/mL) for 3 hours and UV irradiated (120 mJ/cm for 1 minute) 44, or the UV irradiated cells (only) 45, and the PBS control 47 and boosted with the STAV1 after 6 days. Only cells carrying STAVs were able to therapeutically impair the growth of tumors (see FIG. 13A-H).

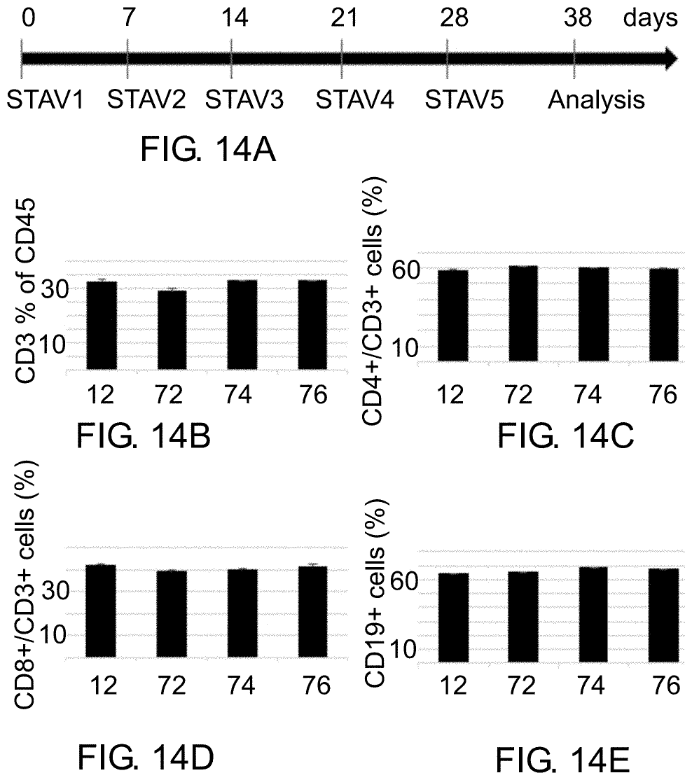

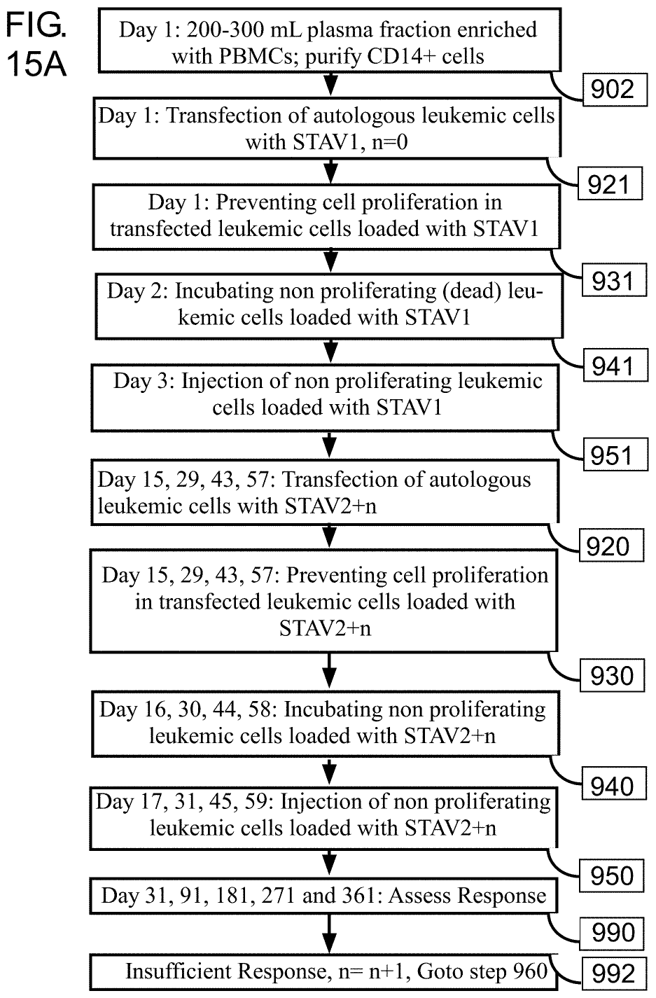

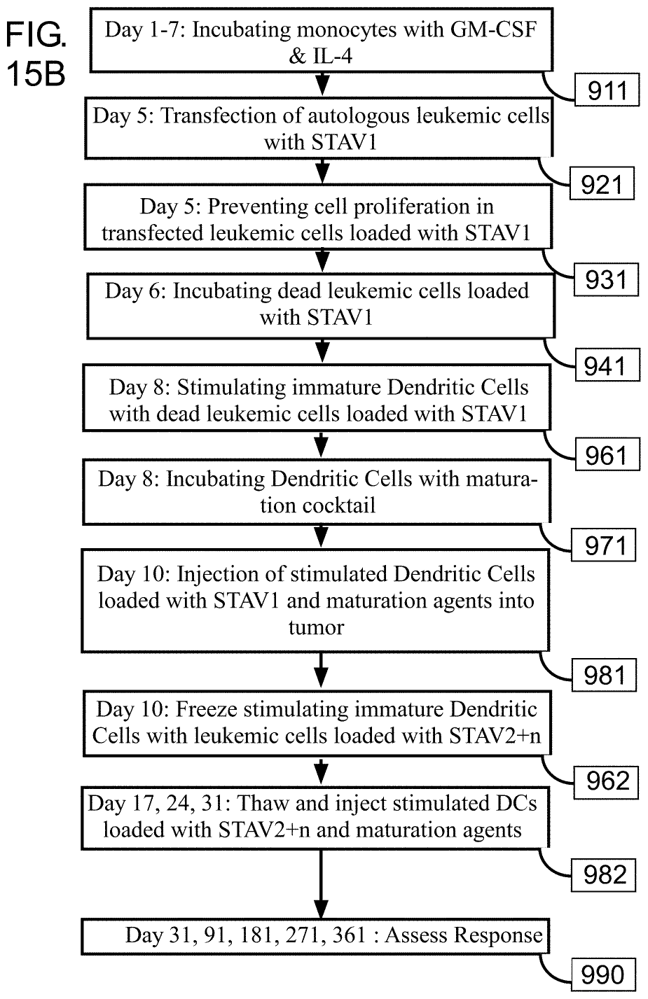

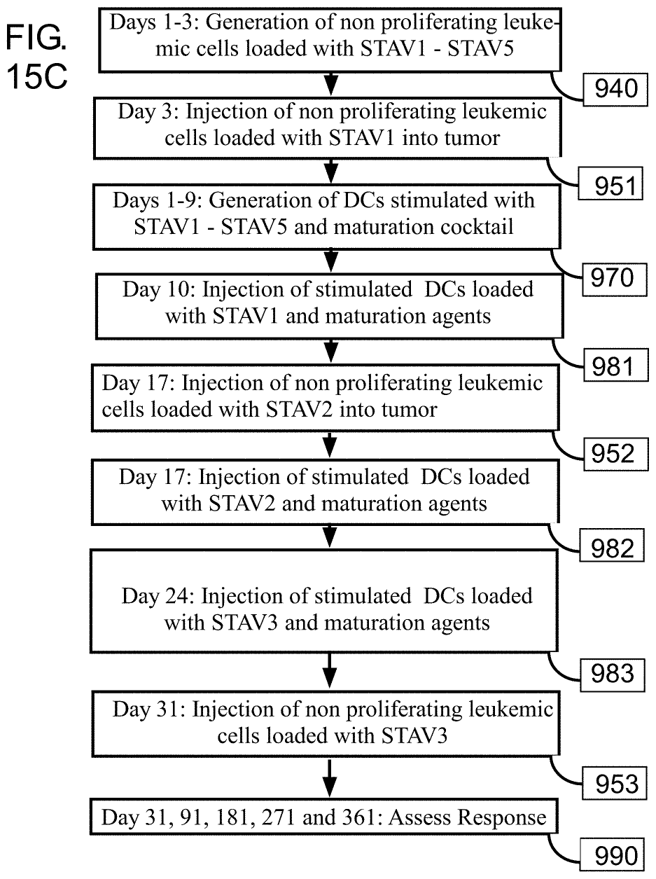

Mouse T-ALL cells (EL4) were transfected with STAVs using the MaxCyte GT system at 4 μg/1×10⁶ cells and irradiated by UV (120 mJ/cm for 1 minute). Mice were i.v. sequentially injected every week with the irradiated T-ALL cells containing STAV1, STAV2, STAV3, STAV4, and STAV5, respectively. Group 1: PBS control (n=3) 12, Group 2: 5×10⁶ cells/mouse (n=3) 72, Group 3: 1×10⁶ cells/mouse (n=3) 74, Group 4: 0.2×10⁶ cells/mouse) (n=3) 76. On day 10 after the last injection, splenocytes were isolated and stained with different fluorescently labeled antibodies (Biolegend: anti-CD3-FITC, anti-CD4-PE, anti-CD8a-PercP, anti-CD45-PacificBlue, anti-CD19-AlexaFluor 700). Flow cytometry analysis was performed using LSR-II. Collectively, the data suggest no auto-immune responses to the therapy. These experiments were replicated using sequential administration of STAVs in the EL4 model and the same results were obtained demonstrating that only EL4 cells carrying STAVs impaired tumor growth (see FIG. 14).

FIG. 14A shows a flow diagram for a protocol of 4 groups (12, 72, 74, and 76), of mice were I.V. sequentially injected every week with the irradiated T-ALL cells (EL4) transfected (72, 74, and 76 with STAV1, STAV2, STAV3, STAV4, and STAV5) using the MaxCyte GT system at 4 μg STAV/1×10⁶ cells and irradiated by UV (120 mJ/cm for 1 minute), where the 4 groups were the PBS control (n=3) 12, 5×10⁶ cells/mouse (n=3) 72, 1×10⁶ cells/mouse (n=3) 74, 2×10⁵ cells/mouse) (n=3) 76. FIG. 14B shows flow cytometry analysis (performed using LSR-II) of the mouse T-ALL cells with anti-CD3-FITC, and anti-CD45-PacificBlue, where the PBS control (n=3) is shown as 12, 5×10⁶ cells/mouse (n=3) 72, 1×10⁶ cells/mouse (n=3) 74, 2×10⁵ cells/mouse) (n=3) 76. FIG. 14C shows flow cytometry analysis (performed using LSR-II) of the mouse T-ALL cells with anti-CD3-FITC, and anti-CD4-PE, where the PBS control (n=3) is shown as 12, 5×10⁶ cells/mouse (n=3) 72, 1×10⁶ cells/mouse (n=3) 74, 2×10⁵ cells/mouse) (n=3) 76. FIG. 14D shows flow cytometry analysis (performed using LSR-II) of the mouse T-ALL cells with anti-CD3-FITC, and anti-CD8a-PercP, where the PBS control (n=3) is shown as 12, 5×10⁶ cells/mouse (n=3) 72, 1×10⁶ cells/mouse (n=3) 74, 2×10⁵ cells/mouse) (n=3) 76. FIG. 14E shows flow cytometry analysis (performed using LSR-II) of the mouse T-ALL cells with anti-CD19-AlexaFluor 700, where the PBS control (n=3) is shown as 12, 5×10⁶ cells/mouse (n=3) 72, 1×10⁶ cells/mouse (n=3) 74, 2×10⁵ cells/mouse) (n=3) 76. On day 38 (10 days after the last injection), splenocytes were isolated and stained with the different fluorescently labeled antibodies.

### Mouse Ex Vivo Experiments

Day 1: Subjects can undergo leukapheresis in order to obtain 200-300 mL plasma fraction enriched with peripheral blood mononuclear cells (PBMCs) for purification of leukemic cells (target yield 2.4×10⁹ cells) and monocytes (target yield 5-30×10⁹ cells).

Transfection of autologous leukemic cells loaded with STAVs. Leukemic cells can be separately transfected (loaded) ex vivo with STAV1, STAV2, STAV3, STAV4, and STAV5, followed by UV irradiation and infused back into subjects on Days 3, 17, 31, 45, and 59, respectively. Five distinct STAVs sequences are shown below (synthesized by Trilink Biotechnologies, HPLC Purified, Endotoxin Tested (<5 EU/mL).

DNA vaccine: Mice were immunized with a plasmid encoding OVA by i.m. electroporation (100 μg per mouse). The booster immunization was given by i.m. two (2) to four (4) weeks after the primary immunization. STING deficient animals (−/−) or controls (+/+) have been twice immunized twice using i.m. electroporation with a DNA vaccine encoding ovalbumin. Serum was measured for anti-OVA IgG. To evaluate if STING played a role in this signaling pathway, STING −/− or control mice were immunized with plasmid DNA encoding the ovalbumin gene. While normal B and T cell subsets were noted in unstimulated STING −/− animals, following immunization Sting −/− mice exhibited significantly less serum ovalbumin (OVA) specific immunoglobulin (Ig)G's compared to controls. In addition, spleen CD8 T-cell frequency and IFN-γ secretion was markedly reduced in Sting mice following immunization, compared to wild type mice. Since immunoglobulin responses to OVA peptide were normal, these data emphasized that the STING-governed DNA sensor pathway is essential for efficient DNA vaccine-induced T-cell responses to antigen. Given this information, it was evaluated whether STING played a role in facilitating T-cell responses following infection with the DNA virus vaccinia that expresses ovalbumin (VV-OVA). This study indicated that control mice, but not Sting (−/−) mice elicited strong T-cell responses to viral encoded OVA, verifying the importance of STING in innate immune signaling processes required for DNA adjuvant activity.

STING also appears important for recognizing DNA's ability to stimulate the innate immune response, including DNA comprising vectors, plasmids, poly dA-dT, poly dC-dG and DNA of varying lengths and sequence composition including ISD. Thus, in another preferred embodiment, STING modulates the innate immune response. It is concluded that STING may play a more predominant role in facilitating RIG-1 mediated innate signaling rather than MDA5. Interestingly, a significant defect was not detected (>5-fold) in the ability of transfected B-form DNA, i.e., poly dA-dT or non CpG containing ISD to induce IFN3 in MEFs lacking STING compared to controls.

### Human Ex Vivo Experiments

The proposed treatment consists of combination of STAVs loaded autologous leukemic cells (up to 5 doses) plus syngeneic STAVs augmented DC cells (up to 4 doses). Treatment-limiting toxicity (TLT) will be assessed over a period of first 60 days, where patients are planned to receive 9 vaccine doses—5 doses of STAVs loaded cells (Days 3, 17, 31, 45, and 59) and 4 doses of DC vaccinations (Days 10, 17, 24, 31).

Eligible subjects with one of the following incurable relapsed/refractory aggressive leukemias: HTLV-1 associated adult T-cell leukemia-lymphoma (ATLL), Acute myelogenous leukemia (AML), and Acute lymphoblastic leukemia (ALL).

For subjects 2 and 3 enrolled of each cohort (ATLL, AML, ALL), each subject will receive dead UV-irradiated STAVs loaded autologous leukemic cells and DC vaccine administration no less than 60 days after the prior enrolled subject 1 has received the second DC vaccination and had no TLTs. After the third subject of each cohort (ATLL, AML, ALL) completes 60-day DLT free observation period, no further staggering is required for that specific disease.

If there are 2 TLTs in the first 3 subjects for each cohort (ATLL, AML, ALL), then study accrual will be held until the protocol is re-evaluated for safety. If two or more subjects cannot have DC vaccine made for technical reasons, then protocol accrual will be held and the protocol and the procedures for manufacture will be evaluated. If subjects progress at any time after receiving the second DC but before they are able to receive further complete treatment as planned on study, then they will be considered evaluable for TLT. If they progress before the second DC administration then they will be replaced for TLT.

The poor prognosis of patients with relapsed/refractory acute leukemia-lymphomas for which no other effective therapies exits is such that it is reasonable to include these patients on an investigational protocol using dead STAVs loaded autologous leukemic cells which appear to be safe in pre-clinical animal models, particularly with DC vaccination, which has been associated with few significant toxicities in human trials. Because completion of study therapy for a given subject can last up to 2 months, in this setting of prolonged treatment it is reasonable to stagger enrollment in the fashion we have described.

STAVs to treat leukemia cells ex vivo followed by re-infusion with autologous DCs stimulated by irradiated autologous UV irradiated (dead) leukemia cells loaded with STAVs. Open-label, phase I study using STAVs to treat leukemia cells ex vivo followed by re-infusion with autologous UV-irradiated (dead) DCs stimulated by irradiated autologous leukemia cells loaded with STAVs.

Day 1: Subjects can undergo leukapheresis in order to obtain 200-300 mL plasma fraction enriched with peripheral blood mononuclear cells (PBMCs) for purification of leukemic cells (target yield 2.4×10⁹ cells) and monocytes (target yield 5-30×10⁹ cells).

Transfection of autologous leukemic cells loaded with STAVs. Leukemic cells can be separately transfected (loaded) ex vivo with STAV1, STAV2, STAV3, STAV4 and STAV5, followed by UV irradiation and infused back into subjects on Days 3, 17, 31, 45, and 59, respectively.

In situ DC maturation and re-infusion. DCs will be generated from monocytes cultured for up to 7 days in the presence of GM-CSF and IL-4, and then loaded with mixture of dead STAVs loaded UV-irradiated leukemic cells on Day 8. In the next step, the immature DCs will be cultured for 48 hours in the presence of maturation agents cocktail consisting of TNF-α, and IL-1s. Then, matured DCs will be injected into subjects on Days 10, 17, 24, and 31.

Response assessment (Day 29+ or −7 days): Subjects with ATLL will be assessed by standard flow cytometry and TCR gene rearrangement studies of peripheral blood, imaging studies (CT scan or CT-PET) (and bone marrow biopsy to confirm complete response only). Subjects with AML will be assessed by standard flow cytometry of peripheral blood (and bone marrow biopsy with cytogenetic studies performed only to confirm complete response [(CR) or CR with incomplete hematologic recovery (CRi)]. Subjects with ALL will be assessed for minimal residual disease by standard flow cytometry of peripheral blood (and bone marrow biopsy to confirm complete response only). Standard PCR for bcr/abl may be used in patients in Philadelphia chromosome positive (Ph+) patients to evaluate molecular response. All patients will be followed monthly for routine monitoring and laboratory tests, and re-assessed for response at the end of months 3, 6, 9 and 12 (+ or −7 days).

Response assessment (1 year): Subjects with a CR (or CRi) after Year 1 who decide to remain on the study will be followed every 3 months (+ or −1 month) for 1 year for routine monitoring and laboratory tests, and response assessments. Subjects who progress after Year 1 will be followed for survival only every 6 months (+ or − 1 month) via a telephone call from years 2 to 5.

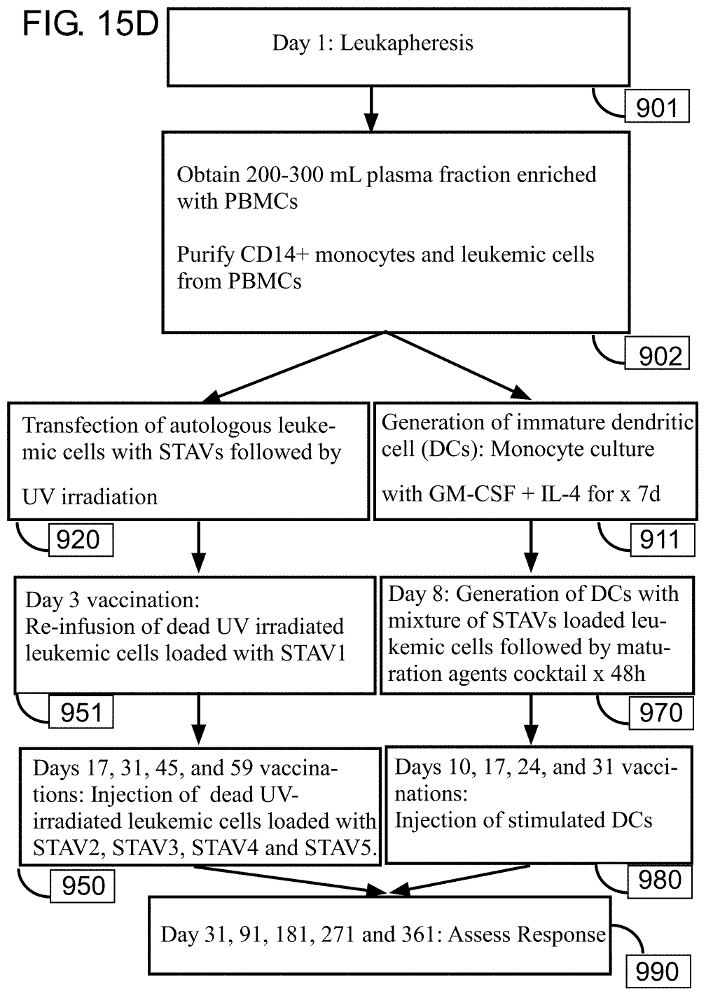

Generation of DCs: DCs can be generated from monocytes cultured for up to 7 days in the presence of GM-CSF and IL-4, see also FIGS. 15B-15D.

FIG. 15A is a flow diagram showing a treatment protocol for treating a patient with cancer where a plurality of 200-300 mL plasma fraction enriched with PBMCs are obtained at day 1 902 from the patient's tumor and can be stored at −20° C., where one of the fractions is thawed and transfected with a STAV (e.g., STAV1) 921, where the transfected cells are irradiated with UV light (approximately 250 nm UV light for between a lower limit of approximately 100 mJ/cm of UV irradiation and an upper limit of approximately 200 mJ/cm of UV irradiation for between a lower limit of approximately 0.1 minute and an upper limit of approximately 10 minutes) or otherwise prevented from proliferating (e.g., x-ray exposure 0.75 Gy/min dose rate, 10-100 min, 50 keV effective energy) 931 and incubated for a period of time (e.g., 24 h) on day 2 941 and injected into the tumor on day 3 951. The procedure is repeated on day 15 with the transfection of a second different STAV, (e.g., STAV2) 920 where the transfected cells are irradiated with UV light 930 and incubated for a period of time (e.g., 24 h) on day 16 940 and injected into the tumor on day 17 950. The procedure can be repeated on day 29 with the transfection of a third different STAV, (e.g., STAV3) 920 where the transfected cells are irradiated with UV light 930 and incubated for a period of time (e.g., 24 h) on day 30 940 and injected into the tumor on day 31 950. The procedure can be repeated on day 43 with the transfection of a fourth different STAV, (e.g., STAV4) 920 where the transfected cells are irradiated with UV light 930 and incubated for a period of time (e.g., 24 h) on day 44 940 and injected into the tumor on day 45 950. The procedure can be repeated on day 57 with the transfection of a fifth different STAV, (e.g., STAV5) 920 where the transfected cells are irradiated with UV light 930 and incubated for a period of time (e.g., 24 h) on day 58 940 and injected into the tumor on day 59 950. The response to the treatment can be assessed on day 31, 91, 181, 271 and 361 990, according to an embodiment of the invention. In an embodiment of the invention, if the response is sufficient the length of time before the next administration of a dead leukemic fraction transfected with a STAV can be extended or delayed.

FIG. 15B is a flow diagram showing a treatment protocol for treating a patient with cancer where a plurality of 200-300 mL plasma fraction enriched with PBMCs are obtained at day 1 from the patient's tumor and can be stored at −20° C. On day 1 through to 7, monocytes are incubated with GM-CSF and IL4 911. On day 1 one of the leukemic cell fractions is thawed and transfected with a STAV (e.g., STAV1) 921, where the transfected cells are irradiated with UV light (or otherwise prevented from proliferating) 931 and incubated for a period of time (e.g., 24 h) on day 2 941 and used to stimulate the immature DCs on day 8 961. On day 8 the stimulated DCs loaded with the STAV are incubated with maturation agents 971. On day 10 the stimulated DCs loaded with the STAV and the maturation agents is injected into the tumor 981. On day 10 stimulated immature DCs loaded with a STAV are frozen (e.g., STAV2-STAV7) 962. On day 17, 24, 31 the stimulated DCs loaded with a STAV (e.g., STAV2-STAV7) and the maturation agent are thawed and injected into the tumor 982. The response to the treatment can be assessed on day 31, 91, 181, 271 and 361 990, according to an embodiment of the invention. In an embodiment of the invention, if the response is sufficient the length of time before the next administration of DCs loaded with a STAV can be extended or delayed.

FIG. 15C is a flow diagram showing a treatment protocol including the protocol of treatment shown in FIG. 15A and the protocol of treatment shown in FIG. 8B. On days 1-3, leukemic cells loaded with a STAV are UV irradiated and incubated for a period of time (e.g., 24 h) 940 and either injected into the tumor on day 3 951 or used to generate the stimulated DC incubated with the maturation cocktail 970. On day 10 the stimulated DC loaded with a STAV and incubated with maturation agents are injected into the tumor 981. On day 17, leukemic cells loaded with a STAV are either injected into the tumor on 952 or used to generate the stimulated DC incubated with the maturation cocktail and are injected into the tumor 982. On day 24 the stimulated DC loaded with a STAV and incubated with maturation agents are injected into the tumor 983. On day 31, leukemic cells loaded with a STAV are injected into the tumor on 953. The response to the treatment can be assessed on day 31, 91, 181, 271 and 361 990, according to an embodiment of the invention. In an embodiment of the invention, if the response is sufficient the length of time before the next administration of a dead leukemic fraction transfected with a STAV can be extended or delayed.

FIG. 15D is a flow diagram showing an alternative treatment protocol for a cancer requiring treatment with a plurality of doses of leukemic cells treated with up to five STAVs comprising the group consisting of STAV1, STAV2, STAV3, STAV4 and STAV5 and a treatment with a plurality of Dendritic Cell vaccines generated with a plurality of STAVs selected from the group consisting of STAV1, STAV2, STAV3, STAV4 and STAV5, according to an embodiment of the invention. On day 1, the leukemic cells are collected 901. On days 10, 17, 24, and 31 injection of thawed stimulated DCs is carried out 980. On days 3, 17, 31, 45, and 59 injection of UV irradiated leukemic cells loaded with STAV1, STAV2, STAV3, STAV4 and STAV5 successively is carried out 980. Obtain 200-300 mL plasma fraction enriched with PBMCs for purification of CD14+ monocytes (target yield 5-30 ˜10⁹ cells) and separation of up to 2.4×10⁹ leukemic cells 902. Fresh leukemic cells (˜3.6×10⁸ will be transfected (loaded) in vitro with STAV1, followed by UV irradiation on Day 3 920. Day 3: i.v. infusion of fresh UV-irradiated (dead) autologous leukemic cells transfected with STAV1 951. On the day of infusion, vital signs will be obtained prior and post infusion for toxicity monitoring purposes. Remaining leukemic cells will be DMSO-frozen in 5 fractions (˜6×10⁸ cells for DC maturation and 3.6×10⁸ cells/vial for subsequent transfection with STAV2, STAV3, STAV4, and STAV5, see 950. At the same time, in vitro culture of autologous monocytes with GM-CSF+IL-4×7 days can be carried out to generate immature DCs 911. Days 8-10: in situ DC maturation: the previously cultured immature DCs (6.0×10⁷ cells) will be stimulated with leukemic cells (6.0×10⁸) loaded with a mixture of STAV1, STAV2, STAV3, STAV4, and STAV5) in the presence of maturation agents cocktail consisting of TNF-α, IL-1s for 48 hours in order to generate mature DCs 970. Days 10, 17, 24, and 31 (+1 day): i.v. injections of stimulated mature DCs (i.e., re-infusion of thawed mature DCs stimulated with UV-irradiated (dead) leukemic cells transfected with mixture of STAV1, STAV2, STAV3, STAV4, and STAV5) 980. On the day of infusion, vital signs will be obtained prior and post infusion for toxicity monitoring purposes. Days 17, 31, 45, and 59 (Day 1+3 days): injections of UV-irradiated (dead) autologous leukemic cells (the yield of 3.6×10⁸ cells after 48-hour culture) transfected two days prior with STAV2, STAV3, STAV4, and STAV5, respectively 950. While on-study subjects will receive up to 5 separate doses of STAVs loaded cells (Days 3, 17, 31, 45, and 59), and up to four (4) DC vaccinations (Days 10, 17, 24, and 31). After therapy completion (approximately 2 months) subjects will be followed at the end months 3, 6, 9, and 12 (+7 days) for clinical assessment (complete physical exam, CBC, CMP, uric acid, phosphorus, and LDH), with response assessments as per Section 9.0 (CT scans, bone marrow biopsy, peripheral blood flow cytometry, and PCR to evaluate for minimal residual disease). Those who remain progression-free after Year 1 and decide to remain on study will be followed approximately every 3-6 months during Year-2 post-treatment for routine monitoring and laboratory tests, and response assessments at the discretion of the investigator. Thereafter, subjects will continue to be followed every 6 months (+1 month) via a telephone call during Years 3 to 5 (at a minimum) for survival only with periodic visits and clinical assessments at the discretion of the investigator. Subjects who discontinue treatment for disease progression will come off treatment and will be followed for survival only every 6 months (+1 month) for up to 5 years from time of treatment initiation. Subjects who withdraw consent will come off study. A study participant is considered to have completed the study once he or she completes all phases of the study treatment and study related laboratory tests. The primary and secondary endpoints will be available for analysis once all patients have met the end points. Therefore, the clinical trial will be considered completed when the last participant has completed all phases of the study including the last visit or the last scheduled procedure shown in the SoA, and the clinical endpoints are available for analysis.

The proposed alternative treatment consists of combination of dead UV-irradiated STAVs loaded with autologous leukemic cells plus DC vaccine. Patients will receive up to 5 separate doses of STAVs loaded cells (Days 3, 17, 31, 45, and 59) 950, and up to four (4) DC vaccinations (Days 10, 17, 24, and 31) 980. The total treatment period is two (2) months. The response is assessed on days 31, 91, 181, 271 and 361 990.

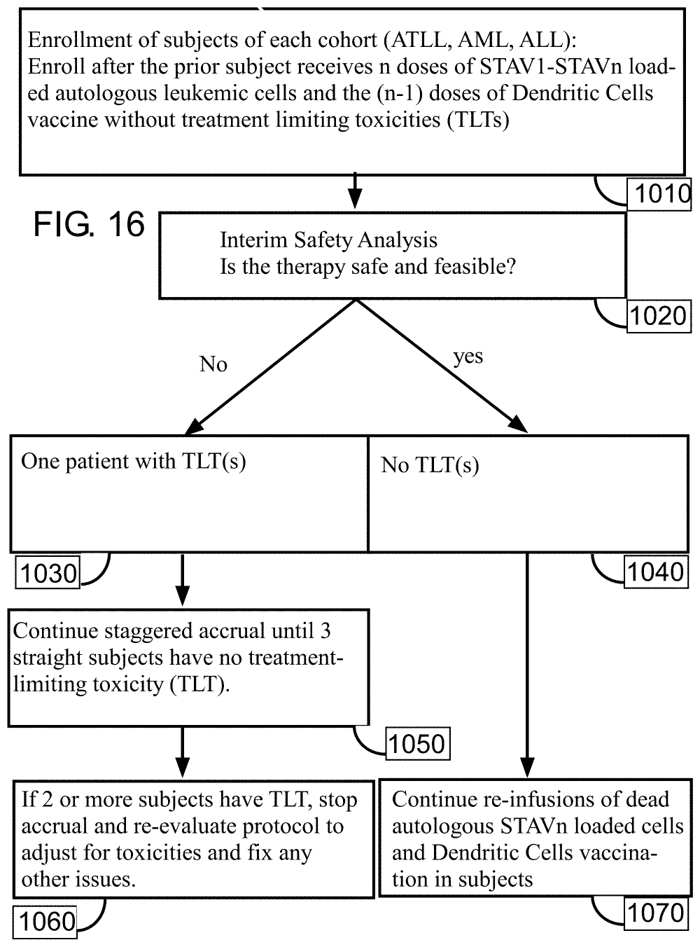

FIG. 16 is a flow diagram showing a limiting toxicity protocol for relapsed/refractory aggressive leukemia. In an embodiment of the present invention, enrollment of subjects of each cohort (ATLL, AML, ALL): enroll after the prior subject receives n doses of STAV1-STAVn loaded autologous leukemic cells and the (n−1) doses of DC vaccine without treatment limiting toxicities (TLTs) 1010. An Interim Safety Analysis is undertaken. 1020. If there is one patient with TLT 1030, then there is one patient with TLT 1030, continue staggered accrual until 3 straight subjects have no treatment-limiting toxicity (TLT) 1050. If two or more subjects have TLT, stop accrual and re-evaluate protocol to adjust for toxicities and fix any other issues 1060. If there are no patients with TLT 1040, then continue injections of dead autologous STAVn loaded cells and/or DC vaccinations in subjects 1070.

Days 3, 17, 31, 45, and 59: Sequential, i.v. infusion of fresh or thawed UV irradiated (dead) syngeneic leukemic cells transfected with five STAVs selected from the group consisting of STAV1, STAV2, STAV3, STAV4, STAV5, STAV6 and STAV7.

Days 7-10: in situ DC maturation. Previously cultured immature DCs can be stimulated (loaded) with mixture of thawed STAVs loaded leukemic cells for 24 hours in the presence of maturation agents cocktail consisting of TNF-α and IL-1s for 48-72 hours in order to generate mature DCs days 10, 17, 24, and 31.

Re-infusion of mixture of thawed mature DCs stimulated with leukemic cells previously transfected with STAV2, STAV3, STAV4, and STAV5 respectively.

Correlative Studies—Molecular evaluations/analysis in patients with HTLV-1/ATLL: Venous blood can be collected from patients diagnosed with leukemia-type HTLV-1/ATLL at baseline, Day 10, at the ends of Months 1, Month 3, Month, 6, Month, 9, Month 12, an at the end-of-treatment visit after early discontinuation. Collected blood specimens can be processed and PMBCs can be isolated by centrifugation using standard Lymphoprep (ficol) procedure. A portion of fresh or thawed cells can be subjected to magnetic CD4-enrichment by negative selection using commercially available kits. These cells can serve as source for protein and RNA after standard extraction procedures. Non-enriched PBMCs can be used to extract genomic DNA for HTLV-1 pro-viral loads. The extracted cells may be utilized fresh or be cryopreserved in DMSO-liquid nitrogen.

Re-infusion of dead STAVs-loaded HTLV-1/ATLL cells can lead to phagocytosis by APCs in vivo. Such event can result in excess indigestible STAVs that can activate STING dependent signaling within APCs which in turn can facilitate a potent anti-tumor T cell activation. In addition, APCs can present HTLV-1 antigens, such as HBZ (which is always expressed ATLL tumors), which can in turn facilitate CTL priming against HTLV-1 infected cells and eliminate such clones.

CTL assays: To evaluate CTL responses after sequential administrations of STAVs loaded tumor cells and DC vaccinations, venous blood can be collected from patients at baseline, before each DC vaccination on Days 10, 17, 24, 31, 45, and at the end of Months 2, 3, and 6. Collected blood specimens can be processed on the same day. PMBCs can be isolated by centrifugation using standard Lymphoprep (ficol) procedure. The extracted cells may be utilized fresh or be cryopreserved in DMSO-liquid nitrogen.

Methods: HTLV-1 specific CTL responses can be assessed using PBMC isolated from peripheral blood. CD8 T cells can be isolated using human MACS CD8+ T cell isolation kit through negative selection (Miltenyil Biotec, 130-096-495). CD8 T Cells can be plated at 2×105 per well and stimulated with 20 μg/ml of tumor cell lysate protein or overlapping 15-aa peptides covering the envelope, TAX or HBZ region of HTLV-1 for ATLL (custom synthesized by GenScript). After 72 hours stimulation IFN gamma secreting cells can be determined using an ELISPOT assay for human IFNγ and quantitated using a ELISPOT reader system. For flow cytometry, cells can be stimulated for 72 hours. Brefeldin A (3 mg/ml) can be added to the cells 6h before analysis. Cells can be then washed, stained with cell surface marker (anti-CD3, anti-CD8), permeabilized with Cytofix/Cytoperm (BD Biosciences), and stained with IFNγ. Data can be acquired using an LSR II flow cytometer.

### B16 Melanoma Model

Sex matched C57/BL6 mice (n=10) inoculated with B16-OVA (5×10⁵) on the flanks. After 7, 10, and 13 days, when tumors are 50 mm³ in volume, 25 μl (4 μg/mL; 0.1 μg/mouse) of Nano-STAVs (STAV1=(SEQ ID NO:24)+(SEQ ID NO:25); STAV2=(SEQ ID NO:26)+(SEQ ID NO:27); or STAV3=(SEQ ID NO:37)+(SEQ ID NO:38)] were injected i.t. in presence or absence of anti-PD-1 or anti-PD-L1 (50 μg/mouse). Nanoparticles alone, checkpoint inhibitor alone, PBS, isotype control antibody were used as controls. The tumor volume was measured using calipers and calculated with the formula V=(length×width²). The generation of anti-tumor CTL activity (against OVA) was measured.

The purity of all compounds was over 95% and was analyzed with Waters LC/MS system. ¹H NMR was obtained at 400 MHz. Chemical shifts are reported relative to dimethyl sulfoxide (6=2.50) for ¹H NMR. Data are reported as (br=broad, s=singlet, d=doublet, t=triplet, q=quartet, m=multiplet).

### Abbreviations

AcOH: acetic acid; AEs: Adverse Events; ALL: Adult Lymphocytic Leukemia; AML: Acute Myeloid Leukemia; ANA: anti nuclear antibody; APC: Antigen Presenting Cells; ATLL: Adult T cell Lymphocytic Leukemia; ATM: atmosphere; BMDM: bone marrow derived macrophages; BP: Blood pressure; BOC₂O: di-tert-butyl dicarbonate; cGAMP: cyclic[G(2′,5′)pA(3′,5′)p]; cGAS: cyclic guanosine monophosphate-adenosine monophosphate synthase; CNS: Central Nervous system; CR: Complete response; CTCAE: Common Terminology Criteria for Adverse Events; CuSO₄: copper sulfate; CDCl₃: deuterated chloroform; CDN: cyclic dinucleotides; CTL: Cytotoxic T cells; DC: dendritic cells; DCM: dichloromethane; DIEA: N,N-diisopropylethylamine; DMA: N,N-dimethylacetamide; DMAP: 4-dimethylaminopyridine; DMF: N,N-dimethylformamide; DMSO: dimethyl sulfoxide; DMSO-d₆: deuterated dimethyl sulfoxide; DOTAP: 1,2-dioleoyl-3-trimethylammonium-propane; dsDNA: double stranded deoxyribonucleic acid; DSPC: distearoylphosphatidylcholine; EoT: End of Treatment; EDCI: 1-ethyl-3-(3-dimethylaminopropyl) carbodiimide; ER: Endoplasmic Reticulum; ESI: electrospray ionization; EtOAc: ethyl acetate; FAM: fluorescein; GMP: good manufacturing practice; H&E: hematoxylin and eosin; HR: Heart rate; HCl: hydrochloric acid; h: hour(s); HPLC: high-performance liquid chromatography; hTERT: immortalized human fibroblasts; HTLV-1: Human T-cell leukemia/lymphoma virus type 1; IFN: interferon; IRF3: Interferon Regulatory Factor 3; IRF7: Interferon Regulatory Factor 7; ISD: Interferon Stimulatory DNA; i.t.: intratumorally; i.v.: intravenous; J: Joule; LCMS: liquid chromatography-mass spectrometry; MS: median survival; MDA5: Melanoma Differentiation associated Antigen 5; mL: milliliter; MeCN: acetonitrile; MHC: Major histocompatibility complex; MEF: Murine Embryonic Fibroblasts; MeOH: methanol; mg: milligram; mmol: millimole; MgSO₄: magnesium sulfate; MHz: megahertz; min: minutes; MS: mass spectrometry; MEF: Murine Embryonic Fibroblasts; Na₂CO₃: sodium carbonate; NaHCO₃: sodium bicarbonate; Nano-STAV a STAV in combination with a lipid nanoparticle; NF-kB: nuclear factor kappa-light-chain-enhancer of activated B cells; NMR: nuclear magnetic resonance; PBMCs: Peripheral blood mononuclear cells; PCR: polymerase chain reaction; s.c.: subcutaneously; SEAP: secreted alkaline phosphatase; STAV: STING-dependent adjuvants; STING: Stimulator of Interferon Genes; Tf: triflate; TKO: TREX1 KnockOut; TM: transmembrane; Pd₂(dba)₃ tris(dibenzylideneacetone)dipalladium(0); Pd(PPh₃)₂Cl₂: bis(triphenylphosphine)palladium(II) dichloride; PAMP: pathogen associated molecular patterns; PE: Physical Exam; ppm: parts per million; polyIC: Polyinosinic:polycytidylic acid; PCR: polymerase chain reaction; PTSA: para-toluene sulfonic acid; qPCR: quantitative real time PCR; QA: Quality Assurance; QC: Quality control; RIG-1: Retinoic acid Inducible Gene 1; RNA: Ribonucleic Acid; RR: Respiratory rate; RT: room temperature; SOPs: Standard operating procedures; SPD: sum of the product of the diameters; STAVS: STING dependent adjuvants, activators; t-BuOH: tert-butanol; TBAF: tetra-n-butylammonium fluoride; TBK1: TANK-binding kinase 1; TCR: T cell receptor; THF: tetrahydrofuran; TRAP: translocon-associated protein; TFA: trifluoroacetic acid; TLR: Toll-like receptors; TLT: Treatment Limiting Toxicity; TMS: trimethylsilane; TLC: thin layer chromatography; TSA: thermal shift assay; μL: microliter; UV: ultraviolet; VSV: vesicular stomatitis virus; WT: Wild Type.

### Example 1

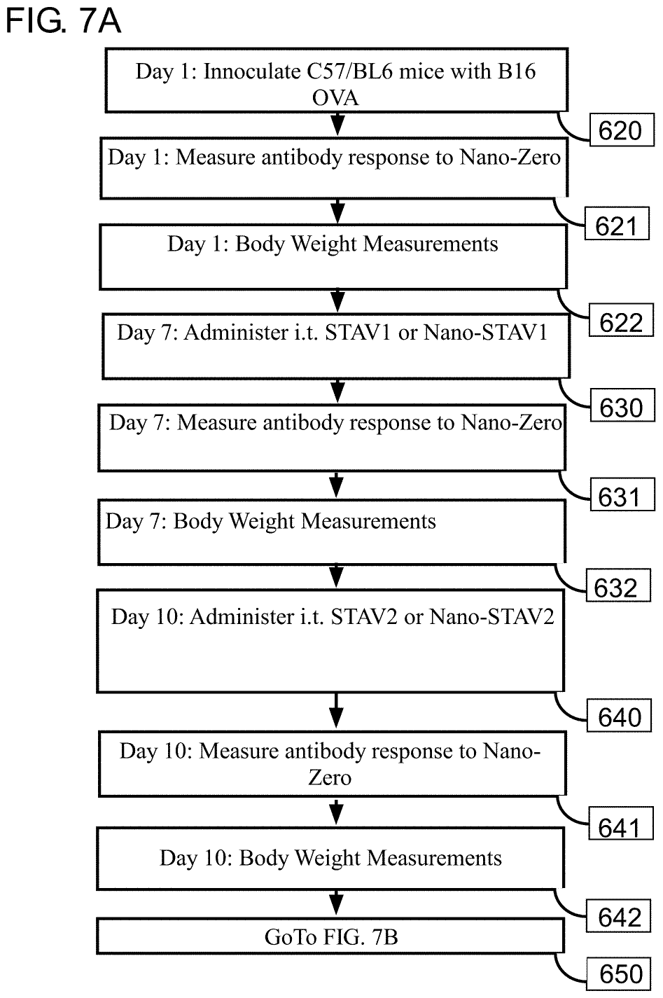

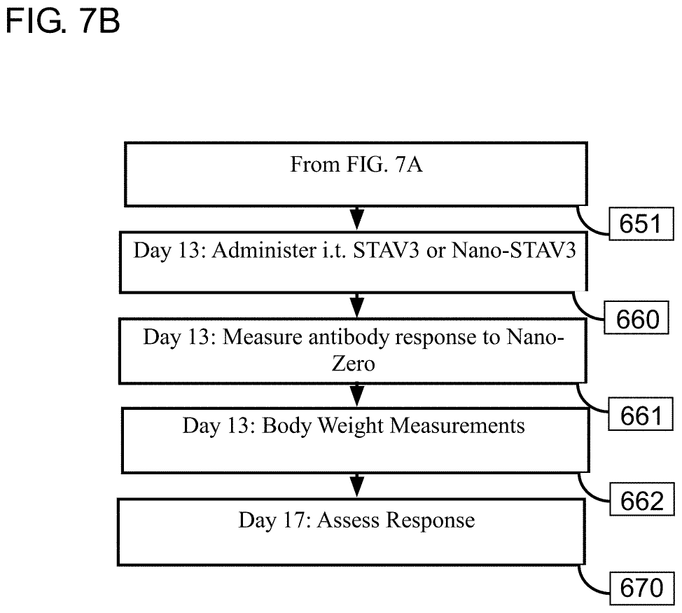

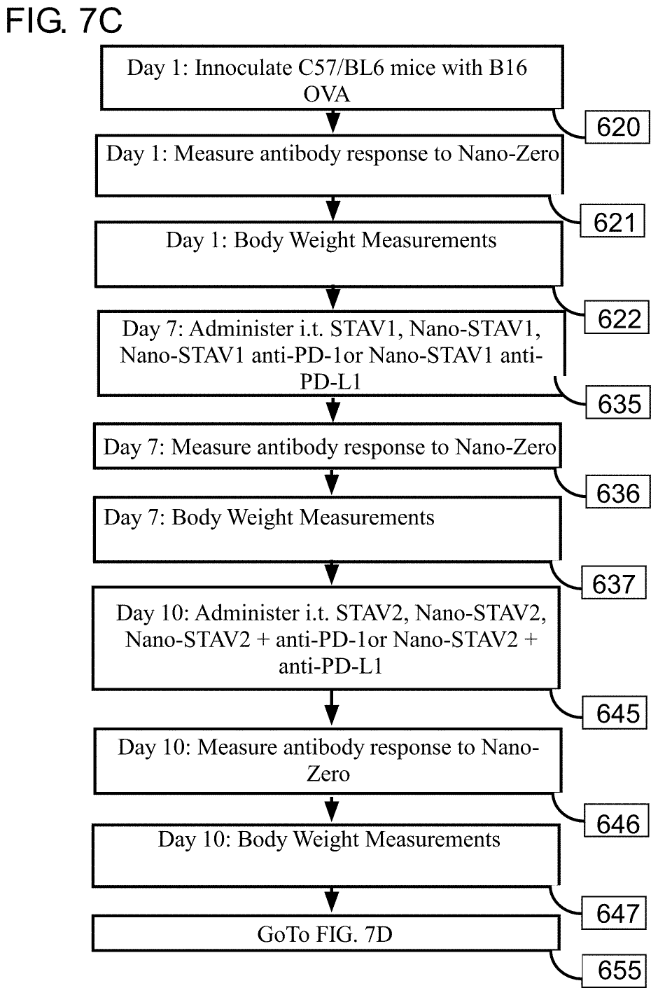

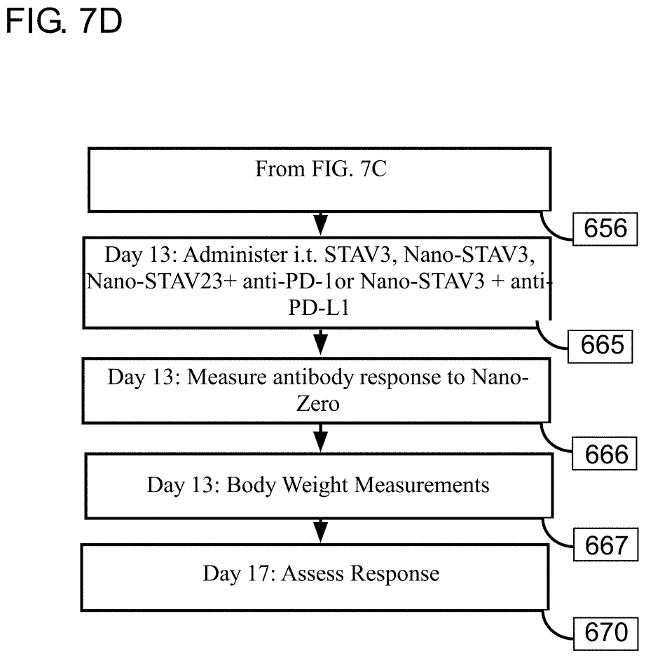

FIGS. 7A and 7B show flow diagrams of the protocol for administration of Nano-STAVs according to an embodiment of the present invention. To examine the importance of dose of STAV C57/BL6 mice (n=10) mice were inoculated on both flanks with B16-OVA (5×10⁵) 620. After seven (7) days, when tumors were 50 mm³ in volume, 25 μl (4 μg/mL; 0.1 ug/mouse) or 25 μL (20 μg/mL; 0.5 μg/mouse for STAV dose escalation examination) of Nano-STAVs (comprising STAV1) was injected (on only one flank) i.t. 630. Three (3) days later 25 μl (4 μg/mL; 0.1 ug/mouse) or 25 μL (20 μg/mL; 0.5 μg/mouse for STAV dose escalation examination) of Nano-STAVs (comprising STAV2) was injected (on the same flank) i.t. 640. Finally, three (3) days later 25 μl (4 μg/mL; 0.1 ug/mouse) or 25 μL (20 μg/mL; 0.5 μg/mouse for STAV dose escalation examination) of Nano-STAVs (comprising STAV3) was injected (on the same flank) i.t. 660. Nanoparticles alone, were used as controls. Body weights were monitored before and after treatment and the tumor volume measured using calipers and calculated with the formula V=(length×width²) 622, 632, 662. The generation of anti-tumor CTL activity (against OVA) was measured using the B16 model 621, 631, 661. Both flanks were monitored. Nano-STAVs generate effective anti-tumor T-cell responses which attack the non-injected tumor on the opposite flank. In addition to these studies, serum taken from the mice, before every inoculation, ascertained the antibody response to the nanoparticles themselves (to gauge the immune response to the formulations) 621, 631, 661. 0.5 μg of the particles was used in solid-phase ELISA assays and the serum from immunized animals incubated at 1/100 for 2 hours in PBS/0.1% Tween. Anti-murine conjugates were used to detect any-anti-Nano-particle antibody. No significant humoral response was observed to the Nano-STAV formulations.

FIGS. 7C and 7D show flow diagrams of the protocol for administration of Nano-STAVs with check point inhibitors according to an alternative embodiment of the present invention. It is known that checkpoint inhibitors can facilitate anti-tumor T cell activity. Nano-STAVs enter the tumor microenvironment (TME) and function by entering and/or adhering to tumor cells. Tumor cells containing Nano-STAVs are engulfed by phagocytes in the TME, to activate extrinsic STING signaling and facilitate the cross-presentation of tumor antigen. Accordingly, stimulating STING signaling is a key mechanism of cytotoxic T cell generation. Anti-PD-L1 and anti-PD1 (IgG BE0091 or anti-PD-L1 BE0101 or anti PD-1 J43 BE0033-2; BioXcell; 50 μg/mouse) was used to evaluate whether Nano-STAVs exert synergistic effects with the checkpoint inhibitors in syngeneic B16 melanoma model. Sex matched C57/BL6 mice (n=10) were inoculated with B16-OVA (5×10⁵) on both flanks 620. After 7, 10, and 13 days, when tumors are 50 mm³ in volume, 25 μl (4 μg/mL; 0.1 μg/mouse) of Nano-STAVs (comprising three or more of STAV1, STAV2, STAV3, STAV4, STAV5, STAV6 and/or STAV7) will be injected i.t. in presence of anti-PD-1 or anti-PD-L1 (50 μg/mouse) 635, 645, 665. Nanoparticles alone, checkpoint inhibitor alone, PBS, isotype control antibody were used as controls. The tumor volume was measured using calipers and calculated with the formula V=(length×width²). Nano-STAVs exhibit potent anti-tumor activity, increasing CTL infiltration within the TME and augment the efficacy of the PD-1/PD-L1 blockade.

### Example 2

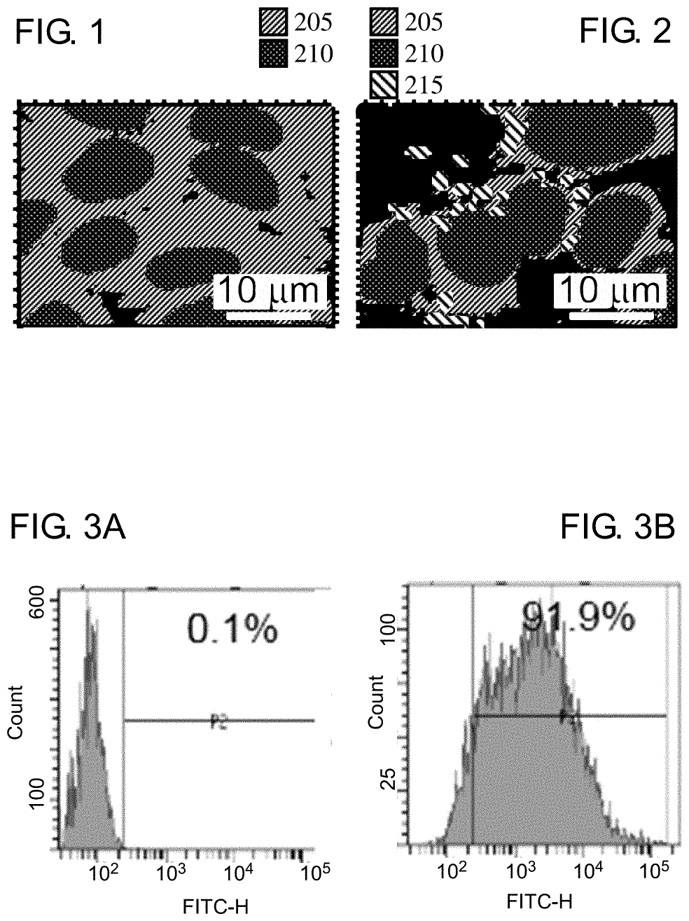

FIG. 1A shows confocal analysis of B16 OVA cells (B16) transfected with no DNA, labeled with FAM (green), DAPI (blue) and anti-calreticulin (red). FIG. 1B shows confocal analysis of B16 OVA cells (B16) transfected with STAVs-FAM, labeled with FAM (green), DAPI (blue) and anti-calreticulin (red). FIG. 2A is a line drawing of FIG. 1A showing confocal analysis of B16 OVA cells (B16) transfected with no DNA labeled with DAPI 205 and anti-calreticulin 210. FIG. 2B is a line drawing of FIG. 1B showing confocal analysis of B16 OVA cells (B16) transfected with STAVs-FAM, labeled with FAM 215, DAPI 205 and anti-calreticulin 210. The STAVs synthetically generated with exonuclease resistant phosphorothioates at the ends (ps) and greater than 70 base pairs were effective at stimulating STING-based cytokine production, regardless of nucleotide content. Accordingly, it is possible to use one STAVs for primary inoculation (AT rich) and a second STAVs for boosting purposes (GC) rich. In an embodiment of the invention, if the STAVs elicited autoimmunity to the STAVs then this can allow a STAV based treatment to avoid autoimmune targeting of the STAV itself. Seven (7) STAVs have been proposed to be used to select five (5) STavS for the purpose of Priming with four (4) boosters available if necessary. FIG. 3A shows flow cytometry analysis of B16 OVA cells (B16) transfected with no DNA. FIG. 3B shows flow cytometry analysis of B16 OVA cells (B16) transfected with STAVs-FAM. As shown in FIG. 1B and FIG. 3B the fluorescent STAVs transfected into B16 melanoma are readily phagocytosed by APC's.

### Example 3

The STAVs can be assembled into lipo-nanoparticles (referred to as Nano-STAVs). However, the STAVs are much more stable than mRNA inserted into LNPs. FIG. 4A shows a Transmission Electron Microscopy image of Nano-Empty LNPs at high magnification. FIG. 4B shows a Transmission Electron Microscopy image of Nano-STAV LNPs at high magnification. FIG. 4C shows a Transmission Electron Microscopy image of Nano-Empty LNPs at low magnification. FIG. 4D shows a Transmission Electron Microscopy image of Nano-STAV LNPs at low magnification. A Nano-STAVs formulated with phospholipid Distearoylphosphatidylcholine, Cholesterol, 4-(dimethylamino)butanoate, and DMG-PEG 2000 has a size of approximately 88 nm.

FIG. 4E shows a plot of cytokine expression for CXCL10 measured with qPCR in WT bone marrow derived mouse macrophages (11=PBS, 32=STAV, 33=STAV+lipofectamine, 34=Nano-Empty, and 35=Nano-STAV). FIG. 4F shows a plot of cytokine expression for CXCL10 measured with qPCR in SKO bone marrow derived mouse macrophages (11=PBS, 32=STAV, 33=STAV+lipofectamine, 34=Nano-Empty, and 35=Nano-STAV). FIG. 4G shows a plot of cytokine expression for CXCL10 measured with qPCR in MAVS KO bone marrow derived mouse macrophages (11=PBS, 32=STAV, 33=STAV+lipofectamine, 34=Nano-Empty, and 35=Nano-STAV). FIG. 4H shows a plot of cytokine expression for IFN3 measured with ELISA in WT bone marrow derived mouse macrophages (11=PBS, 32=STAV, 33=STAV+lipofectamine, 34=Nano-Empty, and 35=Nano-STAV). FIG. 4I shows a plot of cytokine expression for IFN3 measured with ELISA in SKO bone marrow derived mouse macrophages (11=PBS, 32=STAV, 33=STAV+lipofectamine, 34=Nano-Empty, and 35=Nano-STAV). FIG. 4J shows a plot of cytokine expression for IFN3 measured with ELISA in MAVS KO bone marrow derived mouse macrophages (11=PBS, 32=STAV, 33=STAV+lipofectamine, 34=Nano-Empty, and 35=Nano-STAV). In wild-type mouse macrophages, the effect of Nano-STAVs on the antigen-presenting cells (APCs) was characterized by analyzing cytokine expression such as CXCL10 expression by quantitative real time PCR (qPCR) and IFN3 by ELISA. Nano-STAVs were able to strongly induce the expression of CXCL10 (FIG. 4E) and the secretion of IFN3 (FIG. 4H). However, this response was abolished in STING KO (FIG. 4F and FIG. 4I) cells but not in MAVS KO cells (FIG. 4G and FIG. 4J).

FIG. 4K shows a plot of cytokine expression for CXCL10 measured with qPCR in DCs (11=PBS, 32=STAV, 33=STAV+lipofectamine, 34=Nano-Empty, and 35=Nano-STAV). FIG. 4L shows a plot of IFN3 measured with ELISA in DCs (11=PBS, 32=STAV, 33=STAV+lipofectamine, 34=Nano-Empty, and 35=Nano-STAV). In DCs, the Nano-STAVs were able to strongly induce the expression of CXCL10 (FIG. 4K) and the secretion of IFN3 (FIG. 4K), indicating that Nano-STAVs potently activate APCs in vitro through STING-dependent, RLR-independent signaling.

In an embodiment of the present invention, the Nano-STAVS can be formulated with DSPC, Cholesterol, ionizable MC3, and PEG-conjugated lipid at a size of approximately 88 nm (FIGS. 4B and 4D). The effect of Nano-STAVs on the antigen-presenting cells (APCs) was characterized by analyzing CXCL10 cytokine expression using quantitative real time PCR (qPCR) and IFN3 secretion using an ELISA assay.

### Example 4

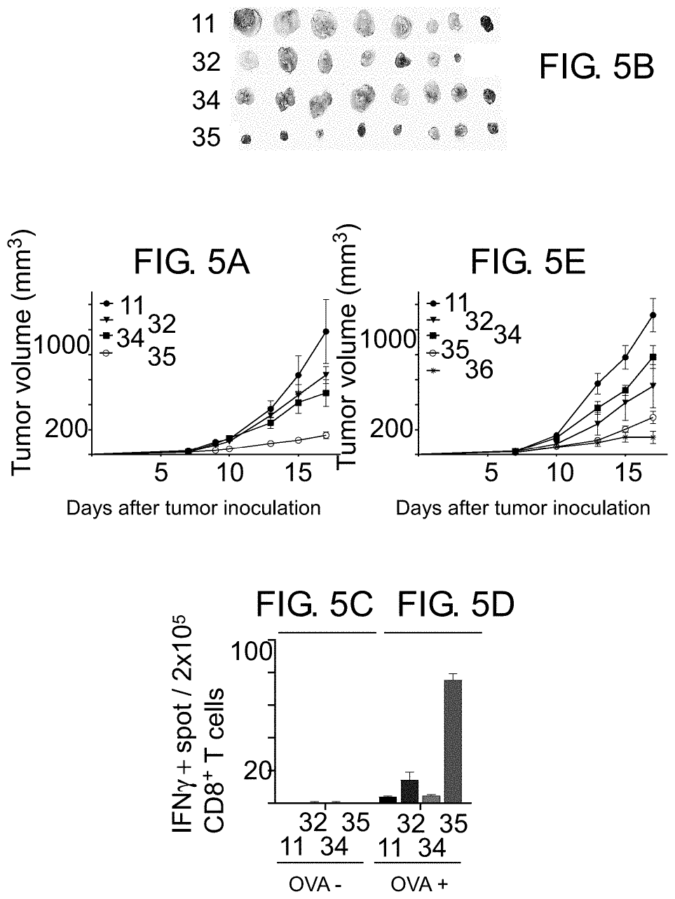

FIG. 5A shows tumor volume of mice s.c. injected with B16 OVA cells (5×10⁵ cells/mouse) on the right flank. On day 7, 10, and 13 after tumor inoculation, the mice were i.t. injected with 11=PBS, 32=STAV, 34=Nano-Empty, and 35=Nano-STAV (0.1 μg/mouse). The tumor volume was measured and calculated with the formula V=(length×width²)/2. At 17 days, the spleen was extracted to measure IFNγ release from CD8+ T cells. FIG. 5B shows digital photographs of mice treated as in FIG. 5A. FIG. 5C shows IFNγ-ELISPOT of (OVA−) mice treated as in FIG. 5A. FIG. 5D shows IFNγ-ELISPOT of (OVA+) mice (i.e., s.c. injected with B16 OVA cells (5×10⁵ cells/mouse) on the right flank) and treated as in FIG. 5A. FIG. 5E shows tumor volume of mice treated as in FIG. 5A or 36=HSV1-γ34.5. Nano-STAVs are highly immunogenic and stimulate APC to generate anti-tumor T cells in vivo as shown by the inoculation of immunocompetent C57BL/6 mice with B16 melanoma expressing OVA (B16 OVA). When the tumors were palpable, the mice were injected i.t. with 11=PBS, 32=naked STAV, 34=Nano-Empty, or 35=Nano-STAV. Tumor sizes were monitored, and the analysis of anti-tumor T cells response was measured by IFNγ-ELISPOT. As shown in FIG. 5A and FIG. 5B the Nano-STAVs greatly reduced the tumor growth compared to PBS control, nanoparticles empty (Nano-Empty), or STAVs alone. Comparison of FIG. 5C and FIG. 5D indicates anti-OVA T cell activity in tumors inoculated with Nano-STAVs. This data indicates that Nano-STAVs are potentially immunogenic compared to naked STAVs or Nano-Empty. Using the B16-OVA model, we compared Nano-STAVs therapy to oncolytic viral T-VEC therapy (using HSV1-γ34.5 as a model) that has been approved for the treatment of advanced melanoma, see FIG. 5E. Both Nano-STAV and HSV1-γ34.5 exert a strong reduction of the tumor growth compared to PBS, nanoparticles empty or STAVs alone (FIG. 5E). However, the Nano-STAV strategy is markedly improved over T-VEC therapy since Nano-STAVs are non-replicative and non-coding.

In an embodiment of the present invention, the Nano-STAVs can be highly immunogenic. In an embodiment of the present invention, the Nano-STAVs can stimulate APC to generate anti-tumor T cells in vivo. Inoculated immunocompetent C57BL/6 mice with B16 melanoma expressing OVA (B16 OVA) were injected i.t. with PBS, naked STAVs, Nano-empty, or Nano-STAVs when the tumors were palpable. Tumor sizes were monitored, and the analysis of anti-tumor T cells response was measured by IFNγ-ELISPOT. Unexpectedly, the Nano-STAVs greatly reduced tumor growth compared to PBS control, nanoparticles empty (Nano-Empty), or STAVs alone (FIGS. 5A, 5B). That an additive effect was observed, where the improvement compared with Mock for the STAVs alone and improvement compared with Mock for the Nano-Empty alone was unexpected. Accordingly, using the Nano-STAVs resulted in a synergistic increase in activity (i.e., decrease in tumor volume). This synergistic effect is also observed in the induction of IFNγ, which is also markedly increased, see FIG. 5C. Accordingly, Nano-STAVs are more immunogenic compared to naked STAVs or Nano-Empty alone.

Using the B16-OVA model, Nano-STAVs therapy was compared with oncolytic viral T-VEC therapy (using HSV1-γ34.5 as a model) that was approved by the FDA in 2015 for the treatment of advanced melanoma. Both Nano-STAV and HSV1-γ34.5 exert a strong reduction of the tumor growth compared to PBS, nanoparticles empty or STAVs alone (FIG. 5D). The Nano-STAV strategy is markedly improved over T-VEC therapy since Nano-STAVs are immunologically inert, non-coding, non-replicative, and therefore safer. Further, Nano-STAVs require less demanding quality control/GMP, and are therefore less expensive to produce.

It is generally thought that T-VEC works by replicating in tumor cells causing the tumor cells to lyse and release tumor antigens At the time of this filling, the working hypothesis is that T-VEC exert its effects through its dsDNA genome stimulating STING signaling in APC's rather than through an oncolytic, replicative activity. This hypothesis is based on the understanding that T-VEC adheres to tumor cells but does not replicate efficiently or lyse them. Following phagocytosis of the tumor cell, the genome of T-VEC (a dsDNA linear genome of approximately 150 kbp) activates STING signaling in the APC, to facilitate tumor cell antigen cross-presentation and the priming of T-cells. STING-deficient mice do not generate anti-tumor T cells following T-VEC treatment, indicating the importance of STING in this process. Since dsDNA of approximately 70 bp is sufficient to activate STING, the Nano-particle delivery of STAVs may exert an effect similar to T-VEC, but significantly more potent and safe.

A Nano-STAV strategy is markedly improved over T-VEC therapy since patients become seropositive against T-VEC (and other viral oncolytics) making the sequential delivery of this therapy ineffective. In contrast, a Nano-STAV strategy can sequentially deliver different STAVs (i.e., prime, boost, boost) since the lipo-material is immunologically inert and the use of the different STAV formulations in our prime-boost strategy avoids auto-immune responses.

Evidence for the efficacy of the Nano-STAVs is also elicited from FIG. 5D which shows no statistical difference between HSV1-γ34.5 and Nano-STAV up until 15 days post treatment and at end of study (17 days) Nano-STAVs were only slightly inferior to treatment with the HSV1-γ34.5 virus. This suggests that induction of IFNγ is an important diagnostic and reaffirms the importance of the synergistic effect observed for the Nano-STAVs induction of IFNγ shown in FIG. 5C.

### Example 5

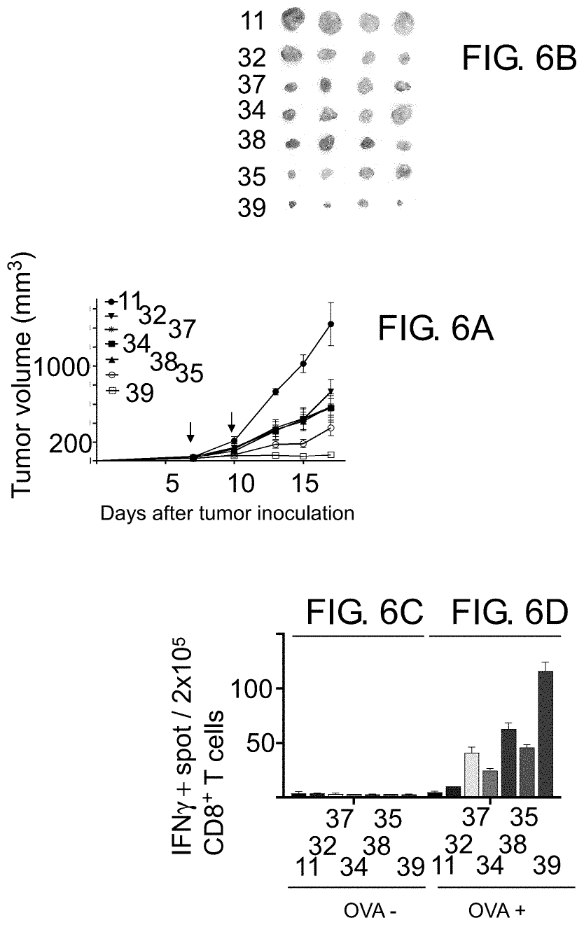

FIG. 6A shows tumor volume of mice treated as in FIG. 5A with 11=PBS, 32=STAV, 37=anti-PD1 (50 μg/mice) administered i.p., 34=Nano-Empty, 38=Nano-Empty+anti-PD1 (50 μg/mice) administered i.p., 35=Nano-STAV (0.1 μg/mouse), and 39=Nano-STAV+anti-PD1 (50 μg/mice) administered i.p. FIG. 6B shows digital photographs of mice treated as in FIG. 6A. FIG. 6C shows IFNγ-ELISPOT of (OVA−) mice treated as in FIG. 6A. FIG. 6D shows IFNγ-ELISPOT of (OVA+) mice (s.c. injected with B16 OVA cells (5×10⁵ cells/mouse) on the right flank) and treated as in FIG. 6A.

Unexpectedly, Nano-STAVs exert greater activity in the presence of checkpoint inhibitors. Palpable tumors were inoculated with Nano-STAV or Nano-Empty as described previously (see FIG. 6). One set of mice received checkpoint therapy alone (50 μg/mouse×2 intraperitoneal injection of anti-PD1; CD279). A second set of mice received checkpoint therapy in combination with Nano-STAVs. A third set of mice received checkpoint therapy with the Nano-Empty (control). Tumor sizes were monitored, and the analysis of anti-tumor T cells responses measured by IFNγ-ELISPOT (see FIG. 6A and FIG. 6B). The Nano-STAVs exhibited robust synergistic anti-tumor activity when used in combination with checkpoint inhibitors when compared to each therapy alone (see FIGS. 6A-6D).

In an embodiment of the present invention, the Nano-STAVS exert potent anti-tumor activity following IT inoculation. In an alternative embodiment of the present invention, the potent anti-tumor activity following IT inoculation is greatly augmented in the presence of checkpoint inhibitors. In an embodiment of the present invention, the approach is cost effective. In an embodiment of the present invention, the approach can be administered sequentially using different STAVs to boost anti-tumor activity. In an alternative embodiment of the present invention, the approach is compatible with and can be used to assist checkpoint inhibitor therapy.

### Example 6

All statistical analysis was performed by Student's t test. The data were considered to be significantly different when P<0.05.

### Further Embodiments

Embodiments contemplated herein include Embodiments P1-P98 following.

Embodiment P1. A composition for treating a human subject suffering from a cancer comprising a Nano-STAV including a double-stranded DNA including a first strand and a second strand, where the first strand comprises at least eighty percent complimentary nucleobases with respect to the second strand, and a lipid nanoparticle including a polymer-conjugated lipid, a sterol, a phospholipid and an ionizing lipid.

Embodiment P2. The composition of Embodiment P1, where the first strand further includes at least one (1) exonuclease resistant phosphorothioate (ps) backbone moiety at the 5′ end and at least one (1) ps backbone moiety at the 3′ end.

Embodiment P3. The composition of Embodiment P1, where the first strand further includes at least three (3) exonuclease resistant phosphorothioate (ps) backbone moieties at the 5′ end and at least three (3) ps backbone moiety at the 3′ end.

Embodiment P4. The composition of Embodiment P1, where the Nano-STAV is selected from the group consisting of STAV1=(SEQ ID NO:24) and (SEQ ID NO:25); STAV2=(SEQ ID NO:26) and (SEQ ID NO:27); STAV3=(SEQ ID NO:37) and (SEQ ID NO:38); STAV4=(SEQ ID NO:39)+(SEQ ID NO:40); STAV5=(SEQ ID NO:41)+(SEQ ID NO:42); STAV6=(SEQ ID NO:43)+(SEQ ID NO:44), and STAV7=(SEQ ID NO:45)+(SEQ ID NO:46).

Embodiment P5. The composition of Embodiment P1, where the polymer-conjugated lipid is DMG-PEG 2000.

Embodiment P6. The composition of Embodiment P1, where the sterol is cholesterol.

Embodiment P7. The composition of Embodiment P1, where the phospholipid is distearoylphosphatidylcholine.

Embodiment P8. The composition of Embodiment P1, where the ionizing lipid is selected from the group consisting of 7-[(2-Hydroxyethyl)[8-(nonyloxy)-8-oxooctyl]amino]heptyl 2-octyldecanoate, N,N-dimethyl-2,3-bis[(9Z)-9-octadecen-1-yloxy]-1-propanamine, N,N-dimethyl-2,2-di-(9Z,12Z)-9,12-octadecadien-1-yl-1,3-dioxolane-4-ethanamine, (6Z,9Z,28Z,31Z)-Heptatriaconta-6,9,28,31-tetraen-19-yl 4-(dimethylamino)butanoate, 4-(dimethylamino)-butanoic acid, (10Z,13Z)-1-(9Z,12Z)-9,12-octadecadien-1-yl-10,13-nonadecadien-1-yl ester, Heptadecan-9-yl 8-{(2-hydroxyethyl)[6-oxo-6-(undecyloxy)hexyl]amino}octanoate, and [(4-Hydroxybutyl)azanediyl]di(hexane-6,1-diyl) bis(2-hexyldecanoate).

Embodiment P9. The composition of Embodiment P1, where the double-stranded DNA comprises between a lower limit of sixty (60) nucleobases, and an upper limit of one hundred and twenty (120) nucleobases.

Embodiment P10. A composition for treating a human subject suffering from a cancer including a Nano-STAV including a double-stranded DNA including a first strand and a second strand, where the first strand comprises at least eighty percent complimentary nucleobases with respect to the second strand, and a lipid nanoparticle including a polymer-conjugated lipid, a sterol, a phospholipid, and an ionizing lipid or a cationic lipid.

Embodiment P11. The composition of Embodiment P10, where the first strand further comprises at least one (1) exonuclease resistant phosphorothioate (ps) backbone moiety at the 5′ end and at least one (1) ps backbone moiety at the 3′ end.

Embodiment P12. The composition of Embodiment P10, where the first strand further comprises at least three (3) exonuclease resistant phosphorothioate (ps) backbone moieties at the 5′ end and at least three (3) ps backbone moiety at the 3′ end.

Embodiment P13. The composition of Embodiment P10, where the Nano-STAV is selected from the group consisting of STAV1=(SEQ ID NO:24) and (SEQ ID NO:25); STAV2=(SEQ ID NO:26) and (SEQ ID NO:27); STAV3=(SEQ ID NO:37) and (SEQ ID NO:38); STAV4=(SEQ ID NO:39)+(SEQ ID NO:40); STAV5=(SEQ ID NO:41)+(SEQ ID NO:42); STAV6=(SEQ ID NO:43)+(SEQ ID NO:44), and STAV7=(SEQ ID NO:45)+(SEQ ID NO:46).

Embodiment P14. The composition of Embodiment P10, where the polymer-conjugated lipid is a polyethylene glycol (PEG)-conjugated lipid.

Embodiment P15. The composition of Embodiment P14, where the PEG-conjugated lipid is selected from the group consisting of DMG-PEG 2000 and DSPE PEG 2000.

Embodiment P16. The composition of Embodiment P10, where the ionizing lipid is selected from the group consisting of 7-[(2-Hydroxyethyl)[8-(nonyloxy)-8-oxooctyl]amino]heptyl 2-octyldecanoate, N,N-dimethyl-2,3-bis[(9Z)-9-octadecen-1-yloxy]-1-propanamine, N,N-dimethyl-2,2-di-(9Z,12Z)-9,12-octadecadien-1-yl-1,3-dioxolane-4-ethanamine, (6Z,9Z,28Z,31Z)-Heptatriaconta-6,9,28,31-tetraen-19-yl 4-(dimethylamino)butanoate, 4-(dimethylamino)-butanoic acid, (10Z,13Z)-1-(9Z,12Z)-9,12-octadecadien-1-yl-10,13-nonadecadien-1-yl ester, Heptadecan-9-yl 8-{(2-hydroxyethyl)[6-oxo-6-(undecyloxy)hexyl]amino}octanoate, and [(4-Hydroxybutyl)azanediyl]di(hexane-6,1-diyl) bis(2-hexyldecanoate).

Embodiment P17. The composition of Embodiment P10, where the cationic lipid is selected from the group consisting of 1,2-dioleoyl-3-trimethylammonium-propane, dimethyldioctadecylammonium bromide, 3β-[N-(N′,N′-dimethylaminoethane)-carbamoyl]cholesterol, dimethyldioctadecylammonium, 1,2-dimyristoyl-3-trimethylammonium-propane, 1,2-stearoyl-3-trimethylammonium-propane and N-(4-carboxybenzyl)-N,N-dimethyl-2,3-bis(oleoyloxy)propan-1-aminium.

Embodiment P18. The composition of Embodiment P10, where the double-stranded DNA comprises between a lower limit of sixty (60) nucleobases, and an upper limit of one hundred and twenty (120) nucleobases.

Embodiment P19. The composition of Embodiment P10, where the first strand comprises at least eighty (80) percent of adenine nucleobases and the second strand comprises at least eighty (80) percent of thymine nucleobases.

Embodiment P20. The composition of Embodiment P10, where the first strand comprises at least eighty (80) percent of cytosine nucleobases and the second strand comprises at least eighty (80) percent of guanine nucleobases.

Embodiment P21. The composition of Embodiment P10, where the double-stranded DNA comprises STAV1=(SEQ ID NO:24)+(SEQ ID NO:25).

Embodiment P22. The composition of Embodiment P10, where the double-stranded DNA comprises STAV2=(SEQ ID NO:26)+(SEQ ID NO:27).

Embodiment P23. The composition of Embodiment P10, where the double-stranded DNA comprises STAV3=(SEQ ID NO:37)+(SEQ ID NO:38).

Embodiment P24. The composition of Embodiment P10, where the double-stranded DNA comprises STAV4=(SEQ ID NO:39)+(SEQ ID NO:40).

Embodiment P25. The composition of Embodiment P10, where the double-stranded DNA comprises STAV5=(SEQ ID NO:41)+(SEQ ID NO:42).

Embodiment P26. The composition of Embodiment P10, where the double-stranded DNA comprises STAV6=(SEQ ID NO:43)+(SEQ ID NO:44).

Embodiment P27. The composition of Embodiment P10, where the double-stranded DNA comprises STAV7=(SEQ ID NO:45)+(SEQ ID NO:46).

Embodiment P28. A composition for treating a human subject suffering from a cancer including a first Nano-STAV, where the first Nano-STAV includes a first STAV selected from the group consisting of STAV1=(SEQ ID NO:24) and (SEQ ID NO:25); STAV2=(SEQ ID NO:26) and (SEQ ID NO:27); STAV3=(SEQ ID NO:37) and (SEQ ID NO:38); STAV4=(SEQ ID NO:39)+(SEQ ID NO:40); STAV5=(SEQ ID NO:41)+(SEQ ID NO:42); STAV6=(SEQ ID NO:43)+(SEQ ID NO:44), and STAV7=(SEQ ID NO:45)+(SEQ ID NO:46), and a LNP including a polymer-conjugated lipid, a sterol, a phospholipid, and an ionizing lipid or a cationic lipid;

a second Nano-STAV, where the second Nano-STAV includes a second STAV selected from the group consisting of STAV1=(SEQ ID NO:24) and (SEQ ID NO:25); STAV2=(SEQ ID NO:26) and (SEQ ID NO:27); STAV3=(SEQ ID NO:37) and (SEQ ID NO:38); STAV4=(SEQ ID NO:39)+(SEQ ID NO:40); STAV5=(SEQ ID NO:41)+(SEQ ID NO:42); STAV6=(SEQ ID NO:43)+(SEQ ID NO:44), and STAV7=(SEQ ID NO:45)+(SEQ ID NO:46), where the second STAV is not the first STAV, and the LNP.

Embodiment P29. The composition of Embodiment P28, where the PEG-conjugated lipid is selected from the group consisting of DMG-PEG 2000 and DSPE PEG 2000.

Embodiment P30. The composition of Embodiment P27, where the sterol is selected from the group consisting of cholesterol, cholesterol sulfate, desmosterol, stigmasterol, lanosterol, 7-dehydrocholesterol, dihydrolanosterol, zymosterol, lathosterol, 14-demethyl-lanosterol, 8(9)-dehydrocholesterol, 8(14)-dehydrocholesterol, FF-MAS, diosgenin, dehydroepiandrosterone (DHEA) sulfate, DHEA, sitosterol, lanosterol-95, zymostenol, sitostanol, campestanol, campesterol, 7-dehydrodesmosterol, pregnenolone, dihydro T-MAS, delta 5-avenasterol, brassicasterol, dihydro FF-MAS, 24-methylene cholesterol, 3β-hydroxy-7-oxo-5-cholestenoic acid, 7α-hydroxy-3-oxo-4-cholestenoic acid, 3β,7α-dihydroxy-5-cholestenoic acid, 3β,7β-dihydroxy-5-cholestenoic acid, 3β-hydroxy-5-cholestenoic acid, 3-oxo-4-cholestenoic acid, 3β,7α,24S-trihydroxy-5-cholestenoic acid, 3β,24S-dihydroxy-5-cholestenoic acid, 3β,7α,25-trihydroxy-5-cholestenoic acid, and 3β,25-OH-7-oxo-5-cholestenoic acid.

Embodiment P31. The composition of Embodiment P28, where the phospholipid is selected from the group consisting of distearoylphosphatidylcholine, phosphatidylcholine, phosphatidylethanolamine, phosphatidylserine, phosphatidic acid, phosphatidylinositol, phosphatidylglycerol, cardiolipin, dipalmitoyl, dimyristoyl, DSPC, dioleoyl, and L-α-phosphatidylcholine.

Embodiment P32. The composition of Embodiment P28, where the ionizing lipid is selected from the group consisting of 7-[(2-Hydroxyethyl)[8-(nonyloxy)-8-oxooctyl]amino]heptyl 2-octyldecanoate, N,N-dimethyl-2,3-bis[(9Z)-9-octadecen-1-yloxy]-1-propanamine, N,N-dimethyl-2,2-di-(9Z,12Z)-9,12-octadecadien-1-yl-1,3-dioxolane-4-ethanamine, (6Z,9Z,28Z,31Z)-Heptatriaconta-6,9,28,31-tetraen-19-yl 4-(dimethylamino)butanoate, 4-(dimethylamino)-butanoic acid, (10Z,13Z)-1-(9Z,12Z)-9,12-octadecadien-1-yl-10,13-nonadecadien-1-yl ester, Heptadecan-9-yl 8-{(2-hydroxyethyl)[6-oxo-6-(undecyloxy)hexyl]amino}octanoate, and [(4-Hydroxybutyl)azanediyl]di(hexane-6,1-diyl) bis(2-hexyldecanoate).

Embodiment P33. The composition of Embodiment P28, where the composition induces Cxcl10 and/or type I IFN cytokine production.

Embodiment P34. The composition of Embodiment P28, further including administering one or more of (i) a third STAV and the LNP, (ii) a fourth STAV and the LNP, and (iii) a fifth STAV and the LNP, where the third STAV, the fifth STAV and the fifth STAV are selected from the group consisting of STAV1=(SEQ ID NO:24) and (SEQ ID NO:25); STAV2=(SEQ ID NO:26) and (SEQ ID NO:27); STAV3=(SEQ ID NO:37) and (SEQ ID NO:38); STAV4=(SEQ ID NO:39)+(SEQ ID NO:40); STAV5=(SEQ ID NO:41)+(SEQ ID NO:42); STAV6=(SEQ ID NO:43)+(SEQ ID NO:44), and STAV7=(SEQ ID NO:45)+(SEQ ID NO:46), where the third STAV is not the second STAV, where the third STAV is not the first STAV, where the fourth STAV is not the first STAV, where the fourth STAV is not the second STAV, where the fourth STAV is not the third STAV, where the fifth STAV is not the first STAV, where the fifth STAV is not the second STAV, where the fifth STAV is not the third STAV, where the fifth STAV is not the fourth STAV.

Embodiment P35. A kit for treating a STING deficiency in a mammal including a Nano-STAV including a STAV selected from the group consisting of STAV1 (SEQ ID NO:24 and SEQ ID NO:25), STAV2 (SEQ ID NO:26 and SEQ ID NO:27), STAV3 (SEQ ID NO:37 and SEQ ID NO:38), STAV4 (SEQ ID NO:39 and SEQ ID NO:40), STAV5 (SEQ ID NO:41 and SEQ ID NO:42), STAV6 (SEQ ID NO:43 and SEQ ID NO:44)), and STAV7=(SEQ ID NO:45)+(SEQ ID NO:46), and a LNP, and instructions for administering the Nano-STAV in the mammal.

Embodiment P36. The kit of Embodiment P35, where the LNP includes a polymer-conjugated lipid, a sterol, a phospholipid, and an ionizing lipid.

Embodiment P37. The kit of Embodiment P36, where the polymer-conjugated lipid is a polyethylene glycol (PEG)-conjugated lipid.

Embodiment P38. The kit of Embodiment P37, where the PEG-conjugated lipid is selected from the group consisting of DMG-PEG 2000 and DSPE PEG 2000.

Embodiment P39. The kit of Embodiment P36, where the sterol is selected from the group consisting of cholesterol, cholesterol sulfate, desmosterol, stigmasterol, lanosterol, 7-dehydrocholesterol, dihydrolanosterol, zymosterol, lathosterol, 14-demethyl-lanosterol, 8(9)-dehydrocholesterol, 8(14)-dehydrocholesterol, FF-MAS, diosgenin, dehydroepiandrosterone (DHEA) sulfate, DHEA, sitosterol, lanosterol-95, zymostenol, sitostanol, campestanol, campesterol, 7-dehydrodesmosterol, pregnenolone, dihydro T-MAS, delta 5-avenasterol, brassicasterol, dihydro FF-MAS, 24-methylene cholesterol, 3β-hydroxy-7-oxo-5-cholestenoic acid, 7α-hydroxy-3-oxo-4-cholestenoic acid, 3β,7α-dihydroxy-5-cholestenoic acid, 3β,7β-dihydroxy-5-cholestenoic acid, 3β-hydroxy-5-cholestenoic acid, 3-oxo-4-cholestenoic acid, 3β,7α,24S-trihydroxy-5-cholestenoic acid, 3β,24S-dihydroxy-5-cholestenoic acid, 3β,7α,25-trihydroxy-5-cholestenoic acid, and 3β,25-OH-7-oxo-5-cholestenoic acid.

Embodiment P40. The kit of Embodiment P36, where the phospholipid is selected from the group consisting of distearoylphosphatidylcholine, phosphatidylcholine, phosphatidylethanolamine, phosphatidylserine, phosphatidic acid, phosphatidylinositol, phosphatidylglycerol, cardiolipin, dipalmitoyl, dimyristoyl, DSPC, dioleoyl, and L-α-phosphatidylcholine.

Embodiment P41. The kit of Embodiment P36, where the ionizing lipid is selected from the group consisting of 7-[(2-Hydroxyethyl)[8-(nonyloxy)-8-oxooctyl]amino]heptyl 2-octyldecanoate, N,N-dimethyl-2,3-bis[(9Z)-9-octadecen-1-yloxy]-1-propanamine, N,N-dimethyl-2,2-di-(9Z,12Z)-9,12-octadecadien-1-yl-1,3-dioxolane-4-ethanamine, (6Z,9Z,28Z,31Z)-Heptatriaconta-6,9,28,31-tetraen-19-yl 4-(dimethylamino)butanoate, 4-(dimethylamino)-butanoic acid, (10Z,13Z)-1-(9Z,12Z)-9,12-octadecadien-1-yl-10,13-nonadecadien-1-yl ester, Heptadecan-9-yl 8-{(2-hydroxyethyl)[6-oxo-6-(undecyloxy)hexyl]amino}octanoate, and [(4-Hydroxybutyl)azanediyl]di(hexane-6,1-diyl) bis(2-hexyldecanoate).

Embodiment P42. A kit for treating a STING deficiency in a mammal including a first Nano-STAV and a second Nano-STAV selected from the group consisting of a first STAV selected from the group consisting of STAV1 (SEQ ID NO:24 and SEQ ID NO:25), STAV2 (SEQ ID NO:26 and SEQ ID NO:27), STAV3 (SEQ ID NO:37 and SEQ ID NO:38), STAV4 (SEQ ID NO:39 and SEQ ID NO:40), STAV5 (SEQ ID NO:41 and SEQ ID NO:42), STAV6 (SEQ ID NO:43 and SEQ ID NO:44), and STAV7=(SEQ ID NO:45)+(SEQ ID NO:46), and a lipid nanoparticle (LNP), and instructions for administering the first Nano-STAV and the second Nano-STAV.

Embodiment P43. The kit of Embodiment P42, where the LNP includes a polymer-conjugated lipid, a sterol, a phospholipid, and an ionizing lipid or a cationic lipid.

Embodiment P44. The kit of Embodiment P43, where the polymer-conjugated lipid is selected from the group consisting of DMG-PEG 2000 and DSPE PEG 2000.

Embodiment P45. The kit of claim 43, where the sterol is selected from the group consisting of cholesterol, cholesterol sulfate, desmosterol, stigmasterol, lanosterol, 7-dehydrocholesterol, dihydrolanosterol, zymosterol, lathosterol, 14-demethyl-lanosterol, 8(9)-dehydrocholesterol, 8(14)-dehydrocholesterol, FF-MAS, diosgenin, dehydroepiandrosterone (DHEA) sulfate, DHEA, sitosterol, lanosterol-95, zymostenol, sitostanol, campestanol, campesterol, 7-dehydrodesmosterol, pregnenolone, dihydro T-MAS, delta 5-avenasterol, brassicasterol, dihydro FF-MAS, 24-methylene cholesterol, 3β-hydroxy-7-oxo-5-cholestenoic acid, 7α-hydroxy-3-oxo-4-cholestenoic acid, 3β,7α-dihydroxy-5-cholestenoic acid, 3β,7β-dihydroxy-5-cholestenoic acid, 3β-hydroxy-5-cholestenoic acid, 3-oxo-4-cholestenoic acid, 3β,7α,24S-trihydroxy-5-cholestenoic acid, 3β,24S-dihydroxy-5-cholestenoic acid, 3β,7α,25-trihydroxy-5-cholestenoic acid, and 3β,25-OH-7-oxo-5-cholestenoic acid.

Embodiment P46. The kit of Embodiment P43, where the phospholipid is selected from the group consisting of phosphatidylcholine, phosphatidylethanolamine, phosphatidylserine, phosphatidic acid, phosphatidylinositol, phosphatidylglycerol, cardiolipin, dipalmitoyl, dimyristoyl, DSPC, dioleoyl, and L-α-phosphatidylcholine.

Embodiment P47. The kit of Embodiment P43, where the ionizing lipid is selected from the group consisting of 7-[(2-Hydroxyethyl)[8-(nonyloxy)-8-oxooctyl]amino]heptyl 2-octyldecanoate, N,N-dimethyl-2,3-bis[(9Z)-9-octadecen-1-yloxy]-1-propanamine, N,N-dimethyl-2,2-di-(9Z,12Z)-9,12-octadecadien-1-yl-1,3-dioxolane-4-ethanamine, (6Z,9Z,28Z,31Z)-Heptatriaconta-6,9,28,31-tetraen-19-yl 4-(dimethylamino)butanoate, 4-(dimethylamino)-butanoic acid, (10Z,13Z)-1-(9Z,12Z)-9,12-octadecadien-1-yl-10,13-nonadecadien-1-yl ester, Heptadecan-9-yl 8-{(2-hydroxyethyl)[6-oxo-6-(undecyloxy)hexyl]amino}octanoate, and [(4-Hydroxybutyl)azanediyl]di(hexane-6,1-diyl) bis(2-hexyldecanoate).

Embodiment P48. The kit of Embodiment P43, where the cationic lipid is selected from the group consisting of 1,2-dioleoyl-3-trimethylammonium-propane, dimethyldioctadecylammonium bromide, 3β-[N-(N′,N′-dimethylaminoethane)-carbamoyl]cholesterol, dimethyldioctadecylammonium, 1,2-dimyristoyl-3-trimethylammonium-propane, 1,2-stearoyl-3-trimethylammonium-propane and N-(4-carboxybenzyl)-N,N-dimethyl-2,3-bis(oleoyloxy)propan-1-aminium.

Embodiment P49. The kit of Embodiment P43, where the instructions further comprise directions on one or both preparing and administering a dendritic cell vaccine.

Embodiment P50. A method for treating a mammal suffering from a cancer including administering a first Nano-STAV and a second Nano-STAV to the mammal, where a time period between administering the first Nano-STAV and the second Nano-STAV is between a minimum of approximately one day, and a maximum of approximately one month.

Embodiment P51. The method of Embodiment P50, where the first Nano-STAV and the second Nano-STAV are selected from the group consisting of STAV1 (SEQ ID NO:24 and SEQ ID NO:25), STAV2 (SEQ ID NO:26 and SEQ ID NO:27), STAV3 (SEQ ID NO:37 and SEQ ID NO:38), STAV4 (SEQ ID NO:39 and SEQ ID NO:40), STAV5 (SEQ ID NO:41 and SEQ ID NO:42), STAV6 (SEQ ID NO:43 and SEQ ID NO:44), and STAV7=(SEQ ID NO:45)+(SEQ ID NO:46).

Embodiment P52. The method of Embodiment P51, further including administering a dendritic cell vaccine to the mammal.

Embodiment P53. A composition for treating a human subject suffering from a cancer including a Nano-STAV, where the Nano-STAV includes a double-stranded DNA, where each strand of DNA comprises at least one (1) exonuclease resistant phosphorothioate backbone moiety at the 5′ end and at least one (1) exonuclease resistant phosphorothioate backbone moiety at the 3′ end, and a LNP including DMG-PEG 2000; cholesterol; distearoylphosphatidylcholine, and (6Z,9Z,28Z,31Z)-Heptatriaconta-6,9,28,31-tetraen-19-yl 4-(dimethylamino)butanoate.

Embodiment P54. The composition of Embodiment P53, where the Nano-STAV comprises a STAV selected from the group consisting of STAV1 (SEQ ID NO:24 and SEQ ID NO:25), STAV2 (SEQ ID NO:26 and SEQ ID NO:27), STAV3 (SEQ ID NO:37 and SEQ ID NO:38), STAV4 (SEQ ID NO:39 and SEQ ID NO:40), STAV5 (SEQ ID NO:41 and SEQ ID NO:42), STAV6 (SEQ ID NO:43 and SEQ ID NO:44), and STAV7=(SEQ ID NO:45)+(SEQ ID NO:46).

Embodiment P55. A method for treating a human subject suffering from a cancer including infusing a plurality of incubated tumor cells loaded with a first STAV into the human subject, and infusing a plurality of mature DCs into the human subject, thereby treating the human subject suffering from the cancer.

Embodiment P56. The method of Embodiment P55, where generating the plurality of incubated tumor cells further comprises generating a plurality of dead tumor cells, where the plurality of dead tumor cells are generated from a plurality of tumor cells treated to prevent cell proliferation.

Embodiment P57. The method of Embodiment P56, where the plurality of tumor cells are treated to prevent cell proliferation by exposing to UV for between lower limit of approximately 10 mJ of UV irradiation, and upper limit of approximately 1 J of UV irradiation.

Embodiment P58. The method of Embodiment P56, where the plurality of tumor cells are treated to prevent cell proliferation by exposing to between a lower limit of approximately 240 nm UV light, and an upper limit of approximately 300 nm UV light, for between a lower limit of approximately 100 mJ/cm of UV irradiation, and an upper limit of approximately 200 mJ/cm of UV irradiation, for between a lower limit of approximately 10⁻¹ minute, and an upper limit of approximately 10′ minutes.

Embodiment P59. The method of Embodiment P55, where generating the plurality of incubated tumor cells further comprises generating a plurality of dead tumor cells, where the plurality of dead tumor cells are generated from a plurality of tumor cells treated by exposing to x-rays.

Embodiment P60. The method of Embodiment P57, where generating the plurality of incubated tumor cells further comprises transfecting the plurality of dead tumor cells with a first STAV to generate the plurality of incubated tumor cells.

Embodiment P61. The method of Embodiment P60, where the first STAV is selected from the group consisting of STAV1 (SEQ ID NO:24 and SEQ ID NO:25), STAV2 (SEQ ID NO:26 and SEQ ID NO:27), STAV3 (SEQ ID NO:37 and SEQ ID NO:38), STAV4 (SEQ ID NO:39 and SEQ ID NO:40), STAV5 (SEQ ID NO:41 and SEQ ID NO:42), STAV6 (SEQ ID NO:43 and SEQ ID NO:44), and STAV7=(SEQ ID NO:45)+(SEQ ID NO:46).

Embodiment P62. The method of Embodiment P60, where the plurality of tumor cells are treated to prevent cell proliferation before transfection with the first STAV.

Embodiment P63. The method of Embodiment P60, where the plurality of tumor cells are treated to prevent cell proliferation after transfection with the first STAV.

Embodiment P64. The method of Embodiment P55, where generating the plurality of mature DCs further comprises generating a plurality of activated DCs, where a plurality of DCs are cultured in the presence of one or more activators to generate the plurality of activated DCs.

Embodiment P65. The method of Embodiment P64, where the one or more activators are selected from the group consisting of GM-CSF, IL-4, TNF-α, and IL-1s..

Embodiment P66. The method of Embodiment P64, where the plurality of DCs are cultured for between a lower limit of approximately 10 hours, and an upper limit of approximately 10 days.

Embodiment P67. The method of Embodiment P64, where generating the plurality of mature DCs further comprises generating a plurality of immature DCs, where the plurality of activated DCs are incubated with a plurality of UV-irradiated leukemic cells loaded with a second STAV to generate the plurality of immature DCs.

Embodiment P68. The method of Embodiment P67, where the plurality of activated DCs are cultured for between a lower limit of approximately 1 hour, and an upper limit of approximately 2 days.

Embodiment P69. The method of Embodiment P67, where the second STAV is selected from the group consisting of STAV1 (SEQ ID NO:24 and SEQ ID NO:25), STAV2 (SEQ ID NO:26 and SEQ ID NO:27), STAV3 (SEQ ID NO:37 and SEQ ID NO:38), STAV4 (SEQ ID NO:39 and SEQ ID NO:40), STAV5 (SEQ ID NO:41 and SEQ ID NO:42), STAV6 (SEQ ID NO:43 and SEQ ID NO:44), and STAV7=(SEQ ID NO:45)+(SEQ ID NO:46).

Embodiment P70. The method of Embodiment P69, where the first STAV is selected as the second STAV.

Embodiment P71. The method of Embodiment P67, where generating the plurality of mature DCs further comprises culturing the plurality of immature DCs in the presence of one or more activators to generate the plurality of mature DCs.

Embodiment P72. The method of Embodiment P71, where the plurality of immature DCs are cultured for between a lower limit of approximately 1 hour, and an upper limit of approximately 7 days.

Embodiment P72. A method for treating a mammal suffering from cancer including administering a first Nano-STAV to the mammal, waiting a time period, and administering a second Nano-STAV to the mammal.

Embodiment P73. The method of Embodiment P71, where the one or more activators are selected from the group consisting of GM-CSF, IL-4, TNF-α, and IL-1s.

Embodiment P74. The method of Embodiment P55, further including a time period between step (a) and step (b) of between a minimum of approximately one day, and a maximum of approximately one month.

Embodiment P75. The method of Embodiment P67, The method of claim 67, where the plurality of UV-irradiated leukemic cells are treated to prevent cell proliferation by exposing to between a lower limit of approximately 240 nm UV light, and an upper limit of approximately 300 nm UV light, for between a lower limit of approximately 100 mJ/cm of UV irradiation, and an upper limit of approximately 200 mJ/cm of UV irradiation, for between a lower limit of approximately 10-1 minute, and an upper limit of approximately 10¹ minutes.

Embodiment P76. The method of Embodiment P64, further including where the plurality of activated DCs are incubated with a plurality of x-ray treated leukemic cells to prevent cell proliferation loaded with a second STAV to generate a plurality of immature DCs.

Embodiment P77. A method for treating a human subject suffering from a cancer including infusing a plurality of first incubated tumor cells loaded with a first STing dependent ActiVator (STAV) into the human subject, infusing a plurality of mature dendritic cells (DCs) into the human subject, and infusing a plurality of second incubated tumor cells loaded with a second STAV into the human subject, thereby treating the human subject suffering from the cancer.

Embodiment P78. The method of Embodiment P77, where the second STAV is not the first STAV.

Embodiment P79. The method of Embodiment P77, where generating the plurality of first incubated tumor cells and/or the plurality of second incubated tumor cells further comprises generating a plurality of dead tumor cells, where the plurality of dead tumor cells are generated from a plurality of tumor cells treated to prevent cell proliferation.

Embodiment P80. The method of Embodiment P79, where the plurality of tumor cells are treated to prevent cell proliferation by exposing to between a lower limit of approximately 240 nm UV light, and an upper limit of approximately 300 nm UV light, for between a lower limit of approximately 100 mJ/cm of UV irradiation, and an upper limit of approximately 200 mJ/cm of UV irradiation, for between a lower limit of approximately 10-1 minute, and an upper limit of approximately 10¹ minutes.

Embodiment P81. The method of Embodiment P79, where generating the plurality of first incubated tumor cells and the plurality of second incubated tumor cells further comprises transfecting the plurality of dead tumor cells with a first STAV and a second STAV to generate a plurality of first transfected tumor cells and a plurality of second transfected tumor cells.

Embodiment P82. The method of Embodiment P81, where the first STAV and the second STAV are selected from the group consisting of STAV1 (SEQ ID NO:24 and SEQ ID NO:25), STAV2 (SEQ ID NO:26 and SEQ ID NO:27), STAV3 (SEQ ID NO:37 and SEQ ID NO:38), STAV4 (SEQ ID NO:39 and SEQ ID NO:40), STAV5 (SEQ ID NO:41 and SEQ ID NO:42), STAV6 (SEQ ID NO:43 and SEQ ID NO:44), and STAV7=(SEQ ID NO:45)+(SEQ ID NO:46), where the first STAV is not the second STAV.

Embodiment P83. The method of Embodiment P81, where generating the plurality of first incubated tumor cells and the plurality of second incubated tumor cells further comprises incubating the plurality of first transfected tumor cells and the plurality of second transfected tumor cells to generate the plurality of first incubated tumor cells and the plurality of second incubated tumor cells.

Embodiment P84. The method of Embodiment P77, where generating the plurality of mature DCs further comprises generating a plurality of activated DCs, where a plurality of DCs are cultured in the presence of one or more activators to generate the plurality of activated DCs.

Embodiment P85. The method of Embodiment P64, where the one or more activators are selected from the group consisting of GM-CSF, IL-4, TNF-α, and IL-1s.

Embodiment P86. The method of Embodiment P84, where the plurality of DCs are cultured for between a lower limit of approximately 10 hours, and an upper limit of approximately 10 days.

Embodiment P87. The method of Embodiment P84, where generating the plurality of mature DCs further comprises generating a plurality of immature DCs, where the plurality of activated DCs are incubated with a plurality of UV-irradiated leukemic cells loaded with a second STAV to generate the plurality of immature DCs.

Embodiment P88. The method of Embodiment P87, where the plurality of activated DCs are cultured for between a lower limit of approximately 1 hour, and an upper limit of approximately 2 days.

Embodiment P89. The method of Embodiment P67, where the second STAV is selected from the group consisting of STAV1 (SEQ ID NO:24 and SEQ ID NO:25), STAV2 (SEQ ID NO:26 and SEQ ID NO:27), STAV3 (SEQ ID NO:37 and SEQ ID NO:38), STAV4 (SEQ ID NO:39 and SEQ ID NO:40), STAV5 (SEQ ID NO:41 and SEQ ID NO:42), STAV6 (SEQ ID NO:43 and SEQ ID NO:44), and STAV7=(SEQ ID NO:45)+(SEQ ID NO:46).

Embodiment P90. The method of Embodiment P89, where the first STAV is selected as the second STAV.

Embodiment P91. The method of Embodiment P87, where generating the plurality of mature DCs further comprises culturing the plurality of immature DCs in the presence of one or more activators to generate the plurality of mature DCs.

Embodiment P92. The method of Embodiment P91, where the plurality of immature DCs are cultured for between a lower limit of approximately 1 hour, and an upper limit of approximately 7 days.

Embodiment P93. The method of Embodiment P91, where the one or more activators are selected from the group consisting of GM-CSF, IL-4, TNF-α, and IL-1s.

Embodiment P94. The method of Embodiment P77, further including a first time period between step (a) and step (b) of between a minimum of approximately one day, and a maximum of approximately one month.

Embodiment P95. The method of Embodiment P94, The method of claim 94, further including a second time period between step (b) and step (c) of between a minimum of approximately one day, and a maximum of approximately one month.

Embodiment P96. The method of Embodiment P87, The method of claim 87, where the plurality of UV-irradiated leukemic cells are treated to prevent cell proliferation by exposing to between a lower limit of approximately 240 nm UV light, and an upper limit of approximately 300 nm UV light, for between a lower limit of approximately 100 mJ/cm of UV irradiation, and an upper limit of approximately 200 mJ/cm of UV irradiation, for between a lower limit of approximately 10⁻¹ minute, and an upper limit of approximately 10¹ minutes

Embodiment P97. A composition for treating a human subject suffering from a cancer including a first STAV, and a second STAV, where the first STAV and the second STAV are selected from the group consisting of STAV1 (SEQ ID NO:24 and SEQ ID NO:25), STAV2 (SEQ ID NO:26 and SEQ ID NO:27), STAV3 (SEQ ID NO:37 and SEQ ID NO:38), STAV4 (SEQ ID NO:39 and SEQ ID NO:40), STAV5 (SEQ ID NO:41 and SEQ ID NO:42), STAV6 (SEQ ID NO:43 and SEQ ID NO:44), and STAV7=(SEQ ID NO:45)+(SEQ ID NO:46).

Embodiment P98. The composition of Embodiment P97, further including a dendritic cell vaccine.

### EQUIVALENTS

Those skilled in the art will recognize, or be able to ascertain using no more than routine experimentation, many equivalents to the specific embodiments and methods described herein. Such equivalents are intended to be encompassed by the scope of the present application.

Example embodiments of the methods, systems, and components of the present invention have been described herein. As noted elsewhere, these example embodiments have been described for illustrative purposes only, and are not limiting. Other embodiments are possible and are covered by the invention. Such embodiments will be apparent to persons skilled in the relevant art(s) based on the teachings contained herein. For example, it is envisaged that, irrespective of the actual shape depicted in the various Figures and embodiments described above, the outer diameter exit of the inlet tube can be tapered or non-tapered and the outer diameter entrance of the outlet tube can be tapered or non-tapered.

Thus, the breadth and scope of the present invention should not be limited by any of the above-described exemplary embodiments, but should be defined only in accordance with the following claims and their equivalents.

What is claimed:

1. A composition for treating a human subject suffering from a cancer with a STing dependent ActiVator (STAV) lipid nanoparticle (LNP) comprising:
a first Nano-STAV comprising:
a) a double-stranded DNA comprising: a first strand and a second strand, where the first strand comprises at least eighty (80) percent complimentary nucleobases with respect to the second strand; and
b) a LNP comprising:
a polymer-conjugated lipid;
a sterol;
a phospholipid; and
an ionizing lipid or a cationic lipid.

2. The composition of claim 1, where one or both the first strand and the second strand further comprise at least one (1) modification located at one or both a 5′ end and a 3′ end.

3. The composition of claim 2, where the modification comprises an exonuclease resistant phosphorothioate backbone moiety.

4. The composition of claim 1, where the first Nano-STAV is selected from the group consisting of the first strand is SEQ ID NO:24 and the second strand is SEQ ID NO:25, the first strand is SEQ ID NO:26 and the second strand is SEQ ID NO:27, the first strand is SEQ ID NO:37 and the second strand is SEQ ID NO:38, the first strand is SEQ ID NO:39 and the second strand is SEQ ID NO:40, the first strand is SEQ ID NO:41 and the second strand is SEQ ID NO:42, the first strand is SEQ ID NO:43 and the second strand is SEQ ID NO:44, and the first strand is SEQ ID NO:45 and the second strand is SEQ ID NO:46.

5. A kit for treating a STING deficiency in a mammal with a STing dependent ActiVator (STAV) lipid nanoparticle (LNP) comprising:
(A) a first Nano-STAV comprising:
(i) a first double-stranded DNA comprising: a first strand and a second strand, where the first strand comprises at least eighty percent complimentary nucleobases with respect to the second strand; and
(ii) a LNP comprising:
a polymer-conjugated lipid;
a sterol;
a phospholipid; and
an ionizing lipid or a cationic lipid;
(B) a second Nano-STAV comprising:
(iii) a second double-stranded DNA comprising: a third strand and a fourth strand, where the third strand comprises at least eighty percent complimentary nucleobases with respect to the fourth strand, where the third strand is not the first strand, where the fourth strand is not the second strand; and
(iv) the LNP; and
(C) instructions for administering the first Nano-STAV and the second Nano-STAV in the mammal.

6. The kit of claim 5, where one or both the first strand and the second strand further comprise at least one (1) modification located at one or both a 5′ end and a 3′ end.

7. The kit of claim 6, where the modification comprises an exonuclease resistant phosphorothioate (ps) backbone moiety.

8. The kit of claim 5, where the first Nano-STAV is selected from the group consisting of the first strand is SEQ ID NO:24 and the second strand is SEQ ID NO:25, the first strand is SEQ ID NO:26 and the second strand is SEQ ID NO:27, the first strand is SEQ ID NO:37 and the second strand is SEQ ID NO:38, the first strand is SEQ ID NO:39 and the second strand is SEQ ID NO:40, the first strand is SEQ ID NO:41 and the second strand is SEQ ID NO:42, the first strand is SEQ ID NO:43 and the second strand is SEQ ID NO:44, and the first strand is SEQ ID NO:45 and the second strand is SEQ ID NO:46.

9. The kit of claim 5, where the second Nano-STAV is selected from the group consisting of the third strand is SEQ ID NO:24 and the fourth strand is SEQ ID NO:25, the third strand is SEQ ID NO:26 and the fourth strand is SEQ ID NO:27, the third strand is SEQ ID NO:37 and the fourth strand is SEQ ID NO:38, the third strand is SEQ ID NO:39 and the fourth strand is SEQ ID NO:40, the third strand is SEQ ID NO:41 and the fourth strand is SEQ ID NO:42, the third strand is SEQ ID NO:43 and the fourth strand is SEQ ID NO:44, and the third strand is SEQ ID NO:45 and the fourth strand is SEQ ID NO:46.

10. A method for treating a mammal suffering from a cancer comprising
(A) administering at a first time a first Nano-STAV comprising:
(i) a first double-stranded DNA comprising: a first strand and a second strand, where the first strand comprises at least eighty percent complimentary nucleobases with respect to the second strand; and
(ii) a LNP comprising:
a polymer-conjugated lipid;
a sterol;
a phospholipid; and
an ionizing lipid or a cationic lipid;
(B) administering at a second time a second Nano-STAV, where the second time is between:
a minimum of approximately one day; and
a maximum of approximately one month after the first time, the second Nano-STAV comprising:
(iii) a second double-stranded DNA comprising: a third strand and a fourth strand, where the third strand comprises at least eighty percent complimentary nucleobases with respect to the fourth strand, where the third strand is not the first strand, where the fourth strand is not the second strand; and
(iv) the LNP.

11. The method of claim 10, where one or both the first strand and the second strand further comprise at least one (1) modification located at one or both a 5′ end and a 3′ end.

12. The method of claim 11, where the modification comprises an exonuclease resistant phosphorothioate backbone moiety.

13. The method of claim 10, where the first Nano-STAV is selected from the group consisting of the first strand is SEQ ID NO:24 and the second strand is SEQ ID NO:25, the first strand is SEQ ID NO:26 and the second strand is SEQ ID NO:27, the first strand is SEQ ID NO:37 and the second strand is SEQ ID NO:38, the first strand is SEQ ID NO:39 and the second strand is SEQ ID NO:40, the first strand is SEQ ID NO:41 and the second strand is SEQ ID NO:42, the first strand is SEQ ID NO:43 and the second strand is SEQ ID NO:44, and the first strand is SEQ ID NO:45 and the second strand is SEQ ID NO:46.

14. The method of claim 10, where the second Nano-STAV is selected from the group consisting of the third strand is SEQ ID NO:24 and the fourth strand is SEQ ID NO:25; the third strand is SEQ ID NO:26 and the fourth strand is SEQ ID NO:27, the third strand is SEQ ID NO:37 and the fourth strand is SEQ ID NO:38, the third strand is SEQ ID NO:39 and the fourth strand is SEQ ID NO:40, the third strand is SEQ ID NO:41 and the fourth strand is SEQ ID NO:42, the third strand is SEQ ID NO:43 and the fourth strand is SEQ ID NO:44, and the third strand is SEQ ID NO:45 and the fourth strand is SEQ ID NO:46.

15. The method of claim 10, further comprising administering one or both a first DC (Dendritic Cell) vaccine and a second DC vaccine to the mammal.

16. The method of claim 15, where the first DC vaccine comprises UV-irradiated leukemic cells loaded with the first nano-STAV and the second DC vaccine comprises UV-irradiated leukemic cells loaded with the second nano-STAV.

17. The method of claim 16, where one or both the first DC vaccine and the second DC vaccine further comprise culturing UV-irradiated leukemic cells with one or more activators.

18. The method of claim 17, where the one or more activators are selected from the group consisting of TNF-α, and IL-1β.

19. The method of claim 16, where the first DC vaccine is administered after the first time and before the second time.

20. The method of claim 16, where the second DC vaccine is administered after the second time.
